(19) United States
(12) Patent Application Publication
(10) Pub. No.: US 2026/0199267 A1
(43) Pub. Date: Jul. 16, 2026

(54) METHOD FOR PREPARING ENDO-CANNABINOID ANALOGUES FROM VEGETABLE OILS USING LIPASE AND USE THEREOF

(71) Applicants: KOREA RESEARCH INSTITUTE OF BIOSCIENCE AND BIOTECHNOLOGY, Daejeon (KR); CELLAPY BIO INC., Daejeon (KR)

(72) Inventors: Jung Hoon SOHN, Daejeon (KR); Hyun Jun KO, Daejeon (KR); Jin-Ku KIM, Daejeon (KR); Jung Hoon BAE, Daejeon (KR); Bong Hyun SUNG, Daejeon (KR); Hyun-Jin KIM, Daejeon (KR); Hyun Joo PARK, Daejeon (KR)

(73) Assignees: KOREA RESEARCH INSTITUTE OF BIOSCIENCE AND BIOTECHNOLOGY, Daejeon (KR); CELLAPY BIO INC., Daejeon (KR)

(21) Appl. No.: 19/136,463

(22) PCT Filed: Dec. 8, 2023
(86) PCT No.: PCT/KR2023/020249

§ 371 Date: Jun. 6, 2025

(30) Foreign Application Priority Data
Dec. 9, 2022 (KR) 10-2022-0171728

(51) Int. Cl. A61K 31/164; A61K 9/00; A61P 11/06; A61P 29/00; A61P 31/04; A61P 35/00; C12N 9/20; C12P 13/02

(52) U.S. Cl. CPC A61K 31/164; A61K 9/0053; A61P 11/06; A61P 29/00; A61P 31/04; A61P 35/00; C12N 9/20; C12P 13/02; C12Y 301/01003

(57) ABSTRACT

The present invention relates to a method for preparing endo-cannabinoid analogues from a vegetable oil using lipase and the use thereof.

### TECHNICAL FIELD

The present invention relates to a method for preparing endo-cannabinoid analogues from a vegetable oil using lipase and the use thereof.

### BACKGROUND ART

The human endo-cannabinoid system (ECS) is a complex cell-signaling system that was discovered in the 1990s while studying the mechanism of action of tetrahydrocannabinol (THC) derived from cannabis. The human ECS transmits signals to cells through cannabinoid receptors, such as CB1, CB2, and TRPV1, which are present in various cells, and is involved in the regulatory systems of various physiological phenomena such as sleep, emotional state, pain, appetite, memory, pregnancy, energy and metabolism, and the immune system.

For this reason, cannabinoid drugs derived from cannabis and hemp are expected to have broad therapeutic applications, including not only the treatment of epilepsy but also anti-cancer effects, dementia treatment, Parkinson's disease treatment, pain relief, fatty liver treatment, and dermatitis treatment. Accordingly, the development of various therapeutics is actively underway. Meanwhile, THC derived from cannabis is restricted in use as a psychoactive substance. However, due to its utility, medical cannabis has been approved for use in various countries, including the United States, Japan, and Thailand. In addition, the Korean Ministry of Food and Drug Safety has facilitated the simplification of procedures for handling cannabis-based pharmaceuticals to improve patient convenience. As such, the diverse medicinal activities of cannabinoids have been identified, highlighting the need for their development as pharmaceutical materials for human use. However, the in vivo degradation mechanism of plant-derived cannabinoids, such as those from cannabis, remains unclear, and while most are excreted through urine, residual components of plant-derived cannabinoids may persist in the body for an extended period, potentially causing various side effects.

In the human body, eicosanoids, including anandamide (N-arachidonoylethanolamide, AEA) and arachidonoylglycerol (2-AG), have been identified as substances that perform functions similar to plant-derived cannabinoids and are referred to as endo-cannabinoids. Unlike plant-derived cannabinoids, endo-cannabinoids have a known in vivo degradation mechanism mediated by fatty acid amide hydrolase (FAAH) or monoacylglycerol lipase (MAGL). Thus, they are considered significantly safer than plant-derived cannabinoids. Therefore, the production and utilization of endo-cannabinoid analogues can provide a safer alternative to plant-derived cannabinoids and enable the development of various applications.

In the body, endo-cannabinoids are biosynthesized from phospholipids present in the cell membrane, with diacylglycerides or N-arachidonic-phosphatidylethanolamine serving as intermediates. This process involves sequential participation of various enzymes, including lipase, phospholipase-D, a/p hydrolase-4, and glycerophosphodiesterase-1. Therefore, there is a need to develop a system for the mass production of endo-cannabinoids, which are highly valuable as pharmaceutical materials for human use.

Meanwhile, lipase is the most commonly used enzyme in biocatalytic reactions, and its biological function is to break down fat (triacylglycerides) into fatty acids and glycerol. Due to the diversity of substrates present in nature, lipase has evolved to exhibit a remarkably broad substrate specificity and catalyzes not only hydrolysis but also esterification, amidation, interesterification (transesterification), aminolysis, transamidation, and perhydrolysis reactions. Additionally, lipase is a highly stable enzyme capable of catalyzing reactions in various liquid environments, including aqueous solutions, organic solvents, ionic liquids, supercritical fluids, and deep eutectic solvents (DES).

Although there are many different types of lipases, Candida antarctica lipase B (CalB) is the most widely studied lipase for organic synthesis in organic solvents. In particular, CalB is an immobilized enzyme that is commercially available as Lipozyme 435, which is a product of the leading global enzyme company, Novozymes. CalB has a molecular weight of 33 kD and a pl of 6.0. It is a typical lipase with a Ser-His-Asp catalytic triad in the a/p configuration; however, its incomplete lid structure results in interfacial activation characteristics that differ from those of typical lipases. Lipozyme 435 is one of the most stable commercially available lipases and is used as a biocatalyst in various reactions, including fat modification and derivative production, biodiesel production, racemic resolution for optical isomer separation, and polymer condensation or degradation reactions. Through previous research, the present inventors have developed the CalB1422-278 series, an improved variant of the CalB enzyme, and have developed a technology for producing oleochemicals using neutral fat or fatty acids (Korean Patent No. 10-2225519).

### DISCLOSURE

### Technical Problem

The present inventors have completed the present invention by employing a lipase enzyme possessing excellent catalytic properties for hydrolysis, esterification, and amidation reactions to prepare endo-cannabinoid analogues from vegetable oils and evaluating various physiological effects of the prepared endo-cannabinoid analogues.

### Technical Solution

It is one object of the present invention to provide a method for preparing endo-cannabinoid analogues, comprising performing an amidation reaction of a vegetable oil, which comprises triglycerides that provide two or more types of fatty acids through hydrolysis, with a C₂₋₆ aminoalcohol using lipase.

It is another object of the present invention to provide a composition for inhibiting the growth of harmful bacteria, comprising a vegetable oil-derived endo-cannabinoid analogues prepared by the method of the present invention as an active ingredient.

It is still another object of the present invention to provide a pharmaceutical composition for preventing or treating a cancer, comprising a vegetable oil-derived endo-cannabinoid analogues prepared by the method of the present invention as an active ingredient.

It is still another object of the present invention to provide a pharmaceutical composition for preventing or treating an inflammatory disease, comprising a vegetable oil-derived endo-cannabinoid analogues prepared by the method of the present invention as an active ingredient.

It is still another object of the present invention to provide a composition for suppressing pain, comprising a vegetable oil-derived endo-cannabinoid analogues prepared by the method of the present invention as an active ingredient.

It is still another object of the present invention to provide a method for preventing or treating a cancer or an inflammatory disease, comprising administering the pharmaceutical composition of the present invention to a subject in need thereof.

It is still another object of the present invention to provide a feed composition comprising a vegetable oil-derived endo-cannabinoid analogues prepared by the method of the present invention as an active ingredient.

It is still another object of the present invention to provide a food composition comprising a vegetable oil-derived endo-cannabinoid analogues prepared by the method of the present invention as an active ingredient.

It is still another object of the present invention to provide a cosmetic composition comprising a vegetable oil-derived endo-cannabinoid analogues prepared by the method of the present invention as an active ingredient.

### Advantageous Effects

The preparation method of the present invention enables the production of endo-cannabinoid analogues from a vegetable oil at a high conversion rate through a simple process involving an enzyme reaction using lipase. Furthermore, a group of endo-cannabinoid analogues prepared by such a method not only exhibit therapeutic effects on cancer and/or inflammatory diseases and relieve pain but also demonstrate no toxicity upon oral administration, making them highly useful as pharmaceutical compositions.

### BRIEF DESCRIPTION OF THE DRAWINGS

FIG. 1 is a diagram illustrating the GC analysis results of the amidation reaction between sunflower oil (SFO) and 3-amino-1-propanol (AP).

FIG. 2 is a diagram illustrating the conversion rate analysis results of the amidation reaction between SFO and AP.

FIG. 3 is a diagram illustrating the GC analysis results of the amidation reaction between SFO and ethanolamine (EA).

FIG. 4 is a diagram illustrating the conversion rate analysis results of the amidation reaction between SFO and EA.

FIG. 5 is a diagram illustrating the GC analysis results of the amidation reaction between SFO and monoisopropanolamine (MIPA).

FIG. 6 is a diagram illustrating the conversion rate analysis results of the amidation reaction between SFO and MIPA.

FIG. 7 is a diagram illustrating the GC analysis results of the amidation reaction between SFO and 2-amino-2-methyl-1-propanol (AMP).

FIG. 8 is a diagram illustrating the conversion rate analysis results of the amidation reaction between SFO and AMP.

FIG. 9 is a diagram illustrating the GC analysis results of the amidation reaction between olive oil (OLO) and AP.

FIG. 10 is a diagram illustrating the conversion rate analysis results of the amidation reaction between OLO and AP.

FIG. 11 is a diagram illustrating the GC analysis results of the amidation reaction between OLO and EA.

FIG. 12 is a diagram illustrating the conversion rate analysis results of the amidation reaction between OLO and EA.

FIG. 13 is a diagram illustrating the GC analysis results of the amidation reaction between coconut oil (CNO) and AP.

FIG. 14 is a diagram illustrating the conversion rate analysis results of the amidation reaction between CNO and AP.

FIG. 15 is a diagram illustrating the GC analysis results of the amidation reaction between CNO and EA.

FIG. 16 is a diagram illustrating the conversion rate analysis results of the amidation reaction between CNO and EA.

FIG. 17 is a diagram illustrating the GC analysis results of the amidation reaction between soybean oil (SBO) and AP.

FIG. 18 is a diagram illustrating the conversion rate analysis results of the amidation reaction between SBO and AP.

FIG. 19 is a diagram illustrating the GC analysis results of the amidation reaction between SBO and EA.

FIG. 20 is a diagram illustrating the conversion rate analysis results of the amidation reaction between SBO and EA.

FIG. 21 is a diagram illustrating the GC analysis results of the amidation reaction between grapeseed oil (GSO) and AP.

FIG. 22 is a diagram illustrating the conversion rate analysis results of the amidation reaction between GSO and AP.

FIG. 23 is a diagram illustrating the GC analysis results of the amidation reaction between evening primrose oil (EPO) and AP.

FIG. 24 is a diagram illustrating the conversion rate analysis results of the amidation reaction between EPO and AP.

FIG. 25 is a diagram illustrating the GC analysis results of the amidation reaction between rapeseed oil (RSO) and AP.

FIG. 26 is a diagram illustrating the conversion rate analysis results of the amidation reaction between RSO and AP.

FIG. 27 is a diagram illustrating the GC analysis results of the amidation reaction between RSO and EA.

FIG. 28 is a diagram illustrating the conversion rate analysis results of the amidation reaction between RSO and EA.

FIG. 29 is a diagram illustrating the GC analysis results of the amidation reaction between meadowfoam seed oil (MFO) and AP.

FIG. 30 is a diagram illustrating the conversion rate analysis results of the amidation reaction between MFO and AP.

FIG. 31 is a diagram illustrating the GC analysis results of the amidation reaction between MFO and EA.

FIG. 32 is a diagram illustrating the conversion rate analysis results of the amidation reaction between MFO and EA.

FIG. 33 is a diagram illustrating the GC analysis results of the amidation reaction between abyssinica seed oil (ABSO) and AP.

FIG. 34 is a diagram illustrating the conversion rate analysis results of the amidation reaction between ABSO and AP.

FIG. 35 is a diagram illustrating the GC analysis results of the amidation reaction between hempseed oil (HSO) and AP.

FIG. 36 is a diagram illustrating the conversion rate analysis results of the amidation reaction between HSO and AP.

FIG. 37 is a diagram illustrating the GC analysis results of the amidation reaction between avocado oil (AVO) and AP.

FIG. 38 is a diagram illustrating the conversion rate analysis results of the amidation reaction between AVO and AP.

FIG. 39 is a diagram illustrating the GC analysis results of the amidation reaction between rice bran oil (RBO) and AP.

FIG. 40 is a diagram illustrating the conversion rate analysis results of the amidation reaction between RBO and AP.

FIG. 41 is a diagram illustrating the GC analysis results of the amidation reaction between shea butter (SHB) and AP.

FIG. 42 is a diagram illustrating the conversion rate analysis results of the amidation reaction between SHB and AP.

FIG. 43 is a diagram illustrating the GC analysis results of the amidation reaction between Aurantiochytrium oil (ATO) and AP.

FIG. 44 is a diagram illustrating the conversion rate analysis results of the amidation reaction between ATO and AP.

FIG. 45 is a diagram illustrating the GC analysis results of the amidation reaction between camellia oil (CAM) and MIPA.

FIG. 46 is a diagram illustrating the conversion rate analysis results of the amidation reaction between CAM and MIPA.

FIG. 47 is a diagram illustrating the GC analysis results of the amidation reaction between safflower oil (SFF) and MIPA.

FIG. 48 is a diagram illustrating the conversion rate analysis results of the amidation reaction between SFF and MIPA.

FIG. 49 is a diagram illustrating the GC analysis results of the amidation reaction between SBO and AMP.

FIG. 50 is a diagram illustrating the conversion rate analysis results of the amidation reaction between SBO and AMP.

FIG. 51 is a diagram illustrating the GC analysis results of the amidation reaction between oleic acid and EA.

FIG. 52 is a diagram illustrating the conversion rate analysis results of the amidation reaction between oleic acid and EA.

FIG. 53 is a diagram illustrating the binding efficiency analysis results of the positive control group, CP55940, to cannabinoid receptor 1 (CB1).

FIG. 54 is a diagram illustrating the binding efficiency analysis results of the positive control group, CP55940, to cannabinoid receptor 2 (CB2).

FIG. 55 is a diagram illustrating the binding efficiency analysis results of SFO-EA, an endo-cannabinoid analogue according to an embodiment of the present invention, to CB1.

FIG. 56 is a diagram illustrating the binding efficiency analysis results of SFO-EA, an endo-cannabinoid analogue according to an embodiment of the present invention, to CB2.

FIG. 57 is a diagram illustrating the binding efficiency analysis results of SFO-AP, an endo-cannabinoid analogue according to an embodiment of the present invention, to CB1.

FIG. 58 is a diagram illustrating the binding efficiency analysis results of SFO-AP, an endo-cannabinoid analogue according to an embodiment of the present invention, to CB2.

FIG. 59 is a diagram illustrating the analysis results of the growth inhibitory effect of SFO-EA, an endo-cannabinoid analogue according to an embodiment of the present invention, on Staphylococcus aureus (Sa).

FIG. 60 is a diagram illustrating the analysis results of the growth inhibitory effect of SFO-EA, an endo-cannabinoid analogue according to an embodiment of the present invention, on Escherichia coli (Ec).

FIG. 61 is a diagram illustrating the analysis results of the growth inhibitory effect of SFO-EA, an endo-cannabinoid analogue according to an embodiment of the present invention, on Salmonella enteritidis (Se).

FIG. 62 is a diagram illustrating the analysis results of the growth inhibitory effect of SFO-EA, an endo-cannabinoid analogue according to an embodiment of the present invention, on Candida albicans (Ca).

FIG. 63 is a diagram illustrating the analysis results of the growth inhibitory effect of SFO-EA, an endo-cannabinoid analogue according to an embodiment of the present invention, on Pseudomonas aeruginosa (Pa).

FIG. 64 is a diagram illustrating the analysis results of the growth inhibitory effect of SFO-AP, an endo-cannabinoid analogue according to an embodiment of the present invention, on Sa.

FIG. 65 is a diagram illustrating the analysis results of the growth inhibitory effect of SFO-AP, an endo-cannabinoid analogue according to an embodiment of the present invention, on Ec.

FIG. 66 is a diagram illustrating the analysis results of the growth inhibitory effect of SFO-AP, an endo-cannabinoid analogue according to an embodiment of the present invention, on Se.

FIG. 67 is a diagram illustrating the analysis results of the growth inhibitory effect of SFO-AP, an endo-cannabinoid analogue according to an embodiment of the present invention, on Ca.

FIG. 68 is a diagram illustrating the analysis results of the growth inhibitory effect of SFO-AP, an endo-cannabinoid analogue according to an embodiment of the present invention, on Pa.

FIG. 69 is a diagram illustrating the cytotoxicity analysis results of SFO-EA, an endo-cannabinoid analogue according to an embodiment of the present invention, toward NIH/3T3 cells.

FIG. 70 is a diagram illustrating the cytotoxicity analysis results of SFO-EA, an endo-cannabinoid analogue according to an embodiment of the present invention, toward HaCaT cells.

FIG. 71 is a diagram illustrating the cytotoxicity analysis results of SFO-AP, an endo-cannabinoid analogue according to an embodiment of the present invention, toward NIH/3T3 cells.

FIG. 72 is a diagram illustrating the cytotoxicity analysis results of SFO-AP, an endo-cannabinoid analogue according to an embodiment of the present invention, toward HaCaT cells.

FIG. 73 is a diagram illustrating the cytotoxicity analysis results of SFO-MIPA, an endo-cannabinoid analogue according to an embodiment of the present invention, toward NIH/3T3 cells.

FIG. 74 is a diagram illustrating the cytotoxicity analysis results of SFO-MIPA, an endo-cannabinoid analogue according to an embodiment of the present invention, toward HaCaT cells.

FIG. 75 is a diagram illustrating the cytotoxicity analysis results of CAM-MIPA, an endo-cannabinoid analogue according to an embodiment of the present invention, toward NIH/3T3 cells.

FIG. 76 is a diagram illustrating the cytotoxicity analysis results of CAM-MIPA, an endo-cannabinoid analogue according to an embodiment of the present invention, toward HaCaT cells.

FIG. 77 is a diagram illustrating the cytotoxicity analysis results of SFF-MIPA, an endo-cannabinoid analogue according to an embodiment of the present invention, toward NIH/3T3 cells.

FIG. 78 is a diagram illustrating the cytotoxicity analysis results of SFF-MIPA, an endo-cannabinoid analogue according to an embodiment of the present invention, toward HaCaT cells.

FIG. 79 is a diagram illustrating the analysis results of the apoptotic effect on lung cancer cells of SFO-EA and SFO-AP, endo-cannabinoid analogues according to an embodiment of the present invention.

FIG. 80 is a diagram illustrating the analysis results of the apoptotic effect on oral cancer cells of SFO-EA and SFO-AP, endo-cannabinoid analogues according to an embodiment of the present invention.

FIG. 81 is a diagram illustrating the analysis results of the apoptotic effect on prostate cancer cells of SFO-EA and SFO-AP, endo-cannabinoid analogues according to an embodiment of the present invention.

FIG. 82 is a diagram illustrating the analysis results of the apoptotic effect on bladder cancer cells of SFO-EA and SFO-AP, endo-cannabinoid analogues according to an embodiment of the present invention.

FIG. 83 is a diagram illustrating the results confirming the inhibition of apoptosis of lung cancer cell induced by CNR2 siRNA transfection.

FIG. 84 is a diagram illustrating the analysis results confirming the inhibitory effect of SFO-EA, an endo-cannabinoid analogue according to an embodiment of the present invention, on NO production.

FIG. 85 is a diagram illustrating the results confirming the inhibitory effect of SFO-AP, an endo-cannabinoid analogue according to an embodiment of the present invention, on NO production.

FIG. 86 is a diagram illustrating the results confirming the inhibitory effect of SFO-MIPA, an endo-cannabinoid analogue according to an embodiment of the present invention, on NO production.

FIG. 87 is a diagram illustrating the results confirming the inhibitory effect of SFO-AMP, an endo-cannabinoid analogue according to an embodiment of the present invention, on NO production.

FIG. 88 is a diagram illustrating the results confirming the inhibitory effect of SFO-MIPA, an endo-cannabinoid analogue according to an embodiment of the present invention, on the production of the inflammatory cytokine IL-1β.

FIG. 89 is a diagram illustrating the results confirming the inhibitory effect of SFO-MIPA, an endo-cannabinoid analogue according to an embodiment of the present invention, on the production of the inflammatory cytokine IL-6.

FIG. 90 is a diagram illustrating the results confirming the inhibitory effect of SFO-MIPA, an endo-cannabinoid analogue according to an embodiment of the present invention, on the production of the inflammatory cytokine TNF-α.

FIG. 91 is a diagram illustrating the results confirming the anti-asthmatic effect of SFO-EA, an endo-cannabinoid analogue according to an embodiment of the present invention.

FIG. 92 is a diagram illustrating the results confirming the pain suppression effect of SFO-EA, an endo-cannabinoid analogue according to an embodiment of the present invention.

FIG. 93 is a diagram illustrating the analysis results by phases confirming the concentration-dependent pain suppression effect of SFO-EA, an endo-cannabinoid analogue according to an embodiment of the present invention.

FIG. 94 is a diagram illustrating the results confirming the effect of SFO-EA, an endo-cannabinoid analogue according to an embodiment of the present invention, on weight gain in animals and improvement in feed efficiency.

### DETAILED DESCRIPTION OF PREFERRED EMBODIMENTS

Each description and embodiment described herein may be applied to other descriptions and embodiments, respectively. That is, all combinations of various elements described herein fall within the scope of the present invention. Further, the scope of the present invention is not limited by the specific description described below.

Furthermore, a person of ordinary skill in the art may recognize or identify numerous equivalents to the specific embodiments of the present invention described herein by using routine experimentation. Moreover, such equivalents are intended to be included within the present invention.

In addition, throughout the specification of the present invention, when it is stated that a part “comprises” a certain component, it means that the part may further include other components, rather than excluding all other components, unless explicitly stated otherwise.

Hereinafter, the present invention will be described in more detail.

In order to achieve the above objectives, the first aspect of the present invention provides a method for preparing endo-cannabinoid analogues, comprising performing an amidation reaction of vegetable oil, which comprises triglycerides that provide two or more types of fatty acids through hydrolysis, with a C₂₋₆ aminoalcohol using lipase.

As used herein, the term “lipase” refers to an enzyme that catalyzes the hydrolysis of fats, breaking down triacylglycerides into fatty acids and glycerol. Due to the diversity of substrates present in nature, lipase has evolved to exhibit a remarkably broad substrate specificity. Some lipases are known to possess functions of promoting esterification, acidolysis, interesterification, transesterification, aminolysis, perhydrolysis, etc. as well as the hydrolysis of fat.

As used herein, the term “amidation” refers to a reaction that provides a compound comprising an amide bond, and the compound thus produced may be a compound known as an organic amide or a carboxamide, which contains the bond represented by R—C(═O)—NR′R″. The amide bond is also referred to as a peptide bond when it is part of a protein backbone. The amidation may mean that the hydroxyl group of a carboxylic acid derivative reacts with the amine group of another compound to form the bond.

As used herein, the term “endo-cannabinoid” refers to a substance that activates a cannabinoid receptor produced in vivo. Since the discovery of the first cannabinoid receptor in 1988, efforts have been made to identify novel endogenous ligands for these receptors. A representative endo-cannabinoid is arachidonoyl ethanolamine (AEA), also known as anandamide, which was the first endo-cannabinoid compound to be discovered. Despite significant structural differences, it exhibits similar pharmacological properties to THC. Anandamide binds to central and peripheral cannabinoid receptors (CB1 and CB2, respectively) and acts as a partial agonist. Examples of other endo-cannabinoids include 2-arachidonyl glyceryl ether (noladin ether), N-arachidonoyl dopamine (NADA), virodhamine (OAE), and the like, and these endo-cannabinoids may bind to CB1 and/or CB2 and act as agonists. Accordingly, compounds that have a structure similar to these endo-cannabinoids in that they comprise a fatty acid at one terminal, and that bind to CB1 and/or CB2 to act as agonists are referred to as endo-cannabinoid analogues.

For example, the endo-cannabinoid analogues provided according to the preparation method of the present invention may be a fatty acid amide (FAA) provided by forming an amide bond between a carboxyl group of a fatty acid and an amine group of a C₂₋₆ aminoalcohol, but is not limited thereto.

As used herein, the term “vegetable oils”, also referred to as plant-based oils, may typically consist of fat components extracted from plants, primarily triglycerides or triacylglycerols (TAG), and may include some diacylglycerols (DAG). Typically, the vegetable oils may be obtained from seeds, nuts, cereal grains, and fruits. The term “vegetable oils” may narrowly refer to only oils among these substances that remain in a liquid state at room temperature, but may broadly refer to the entire range of triglycerides extracted from plants, regardless of their physical properties. These vegetable oils may be a mixture comprising various saturated or unsaturated fatty acids, and the types and contents of fatty acids that consist thereof may differ depending on the plant type.

In one example, sunflower oil, olive oil, and soybean oil all include C16 or C18 saturated or unsaturated fatty acids, such as linoleic acid, oleic acid, linolenic acid, stearic acid, and palmitic acid. However, the contents of each component in these oils vary. The fatty acid composition of sunflower oil is 59% linoleic acid, 30% oleic acid, 6% stearic acid, and 5% palmitic acid; the fatty acid composition of olive oil is 71% oleic acid, 13% palmitic acid, 12% linoleic acid, and 4% stearic acid; and the fatty acid composition of soybean oil is 54% linoleic acid, 24% oleic acid, 11% palmitic acid, 7% linolenic acid, and 4% stearic acid. Meanwhile, coconut oil includes relatively short-chain saturated or unsaturated fatty acids, specifically C10, C12, C14, and C16. Its fatty acid composition consists of 47% lauric acid, 18% myristic acid, 9% palmitic acid, 6% capric acid, 6% oleic acid, 3% stearic acid, and 2% linoleic acid. Abyssinica seed oil, on the other hand, contains relatively long-chain saturated or unsaturated fatty acids, specifically C18, C20, and C22. Its fatty acid composition consists of 63.8% erucic acid, 15.1% oleic acid, 13.2% linoleic acid, 2.4% eicosenoic acid, and 2.1% behenic acid.

For example, the vegetable oils that may be used in the method for producing the endo-cannabinoid analogues of the present invention may be one or more selected from the group consisting of sunflower oil (SFO), olive oil (OLO), coconut oil (CNO), soybean oil (SBO), grapeseed oil (GSO), evening primrose oil (EPO), rapeseed oil (RSO), meadowfoam seed oil (MFO), abyssinica seed oil (ABSO), hempseed oil (HSO), avocado oil (AVO), rice bran oil (RBO), shea butter (SHB), Aurantiochytrium oil (ATO), camellia oil (CAM), safflower oil (SFF), palm oil, macadamia nut oil, mustard oil, and castor oil, but is not limited thereto.

As used herein, the term “triglyceride” refers to a compound comprising three fatty acids esterified to a glycerol backbone. While the correct chemical designation is “triacylglycerol (TAG)”, it is more commonly referred to as “triglyceride”. Triglycerides are a main dietary fat, which is hydrolyzed into fatty acids and monoglycerides by lipase in the intestine.

As used herein, the term “fatty acids” is a term used in the field of chemistry, especially in the field of biochemistry, and refers to carboxylic acids having a long saturated or unsaturated aliphatic chain. Most naturally occurring fatty acids may include a straight-chain structure without branches, consisting of an even number of carbon atoms ranging from 4 to 28. These fatty acids are typically present in the form of three main classes of esters—triglycerides, phospholipids, and cholesteryl esters—rather than existing as free fatty acids in living organisms. In these esterified forms, fatty acids serve as essential energy sources and structural components of cells.

For example, the two or more fatty acids contained in the vegetable oil may be a mixture of two or more selected from the group consisting of linoleic acid, linolenic acid, oleic acid, stearic acid, palmitic acid, myristic acid, lauric acid, capric acid, eicosenoic acid, erucic acid, docosadienoic acid, behenic acid, docosahexaenoic acid, eicosapentaenoic acid, arachidonic acid, dodecanoic acid, hexanoic acid, caproic acid, tripalmitin, tricaproin, and tricaprin, but is not limited thereto.

As used herein, the term “aminoalcohol” refers to an organic compound including both a hydroxyl group (—OH) and an amino group (amino, —NH2, —NHR, or —NR2) in the alkane backbone. The alkane may have a straight or branched chain structure.

For example, the C₂₋₆ aminoalcohol is one or more selected from the group consisting of 3-amino-1-propanol (AP), ethanolamine (EA), monoisopropanolamine (MIPA), 2-amino-2-methyl-1-propanol (AMP), 3-amino-1,2-propanediol, and serinol (2-aminopropane-1,3-diol), but is not limited thereto.

For example, in the preparation method of the present invention, the lipase may be added at a ratio of 0.01% to 30% (w/v) relative to the vegetable oil. Specifically, it may be added at a ratio of 0.01% to 10%, 0.1% to 10%, 0.05% to 5%, 0.1% to 5%, 0.5% to 5%, 0.5% to 3%, or 0.5% to 2%, but the ratio is not limited thereto, provided that the lipase exhibits catalytic activity in the reaction.

For example, in the preparation method of the present invention, the C₂₋₆ aminoalcohol may be used in a molar ratio of 1:1 to 1:3 relative to the vegetable oil. In general, considering that most vegetable oils are composed of triglycerides, which can provide three fatty acid molecules from a single molecule through hydrolysis, a molar ratio of at least 1:1, for example, 1:3, may be used to ensure a sufficient reaction. However, the ratio is not limited thereto.

For example, the amidation is performed at 40° C. to 70° C. for 15 to 200 hours, but is not limited thereto. Specifically, the amidation reaction is carried out through the enzymatic catalysis of lipase and may be performed at a suitable temperature. The reaction time may be adjusted depending on the type of substrate and other reaction conditions. For example, the reaction may be performed at 40° C. to 60° C., 50° C. to 70° C., or 50° C. to 60° C., and may be carried out for 10 to 50 hours, 100 to 200 hours, 60 to 120 hours, or 80 to 100 hours. However, the reaction conditions are not limited thereto.

For example, the preparation method of the present invention may be carried out with a conversion of 85% to 99%, 88% to 98%, or 90% to 97.5%.

The second aspect of the present invention provides a composition for inhibiting the growth of harmful bacteria, comprising a vegetable oil-derived endo-cannabinoid analogues prepared by the method of the first aspect as an active ingredient.

For example, the harmful bacteria whose growth can be inhibited using the composition of the present invention may be Staphylococcus aureus, Escherichia coli, Salmonella enteritidis, or Candida albicans. In a specific embodiment of the present invention, when vegetable oil-derived endo-cannabinoid analogues prepared according to the present invention were administered, it was observed that at concentrations of 0.1 ppm or higher, or 1 ppm or higher, the growth of Staphylococcus aureus, Escherichia coli, Salmonella enteritidis, and/or Candida albicans was inhibited in a concentration-dependent manner, while no growth inhibitory effect was observed against Pseudomonas aeruginosa within the tested concentration range.

The third aspect of the present invention provides a composition for preventing or treating a cancer, comprising a vegetable oil-derived endo-cannabinoid analogues prepared by the method of the first aspect as an active ingredient.

As used herein, the term “prevention” refers to any activity inhibiting or delaying the occurrence, spread, and recurrence of a cancer through administering the composition of the present invention, and the term “treatment” refers to any activity that improves or beneficially changes a symptom of the disease by administering the composition of the present invention.

The cancer that may be prevented or treated by administering the pharmaceutical composition of the present invention may be lung cancer, oral cancer, prostate cancer, bladder cancer, colorectal cancer, colon cancer, cholangiocarcinoma (bile duct carcinoma), gastric cancer, breast cancer, pancreatic cancer, ovarian cancer, placental cancer, choriocarcinoma, esophageal squamous cell cancer, glioblastomas, melanomas, renal carcinomas, cervical squamous cell carcinomas, hepatocellular carcinomas, or cervical intraepithelial neoplasia, but is not limited thereto.

In a specific embodiment of the present invention, it was confirmed that the vegetable oil-derived endo-cannabinoid analogues prepared by the method of the present invention exhibited an apoptotic effect on lung cancer cells, oral cancer cells, prostate cancer cells, and/or bladder cancer cells.

The composition of the present invention may further comprise pharmaceutically acceptable carriers, diluents, or excipients, and may be formulated into various forms such as powders, granules, tablets, capsules, suspensions, emulsions, syrups, aerosols, etc., according to conventional methods for each intended use. It can be administered orally or through various routes including intravenous, intraperitoneal, subcutaneous, rectal, and local administration. Examples of suitable carriers, excipients, or diluents that may be included in such composition include lactose, dextrose, sucrose, sorbitol, mannitol, xylitol, erythritol, maltitol, starch, acacia gum, alginate, gelatin, calcium phosphate, calcium silicate, cellulose, methylcellulose, amorphous cellulose, polyvinylpyrrolidone, water, methyl hydroxybenzoate, propyl hydroxybenzoate, talc, magnesium stearate, and mineral oil. Additionally, the composition of the present invention may further include fillers, anti-coagulants, lubricants, moisturizing agents, fragrances, emulsifiers, preservatives, etc.

Solid preparations for oral administration include tablets, pills, powders, granules, capsules, etc., and these solid preparations are formulated by mixing the composition with at least one or more excipients, such as starch, calcium carbonate, sucrose, lactose, and gelatin. Further, in addition to simple excipients, lubricants such as magnesium stearate and talc may be used.

Examples of oral liquid preparations may include suspensions, solutions, emulsions, syrups, etc., and in addition to commonly used simple diluents such as water and liquid paraffin, various excipients such as wetting agents, sweeteners, flavoring agents, and preservatives may be included.

Preparations for non-oral administration include sterilized aqueous solutions, non-aqueous solutions, suspensions, emulsions, freeze-dried preparations, and suppositories. As non-aqueous solutions and suspending agents, propylene glycol, polyethylene glycol, vegetable oils such as olive oil, injectable esters such as ethyl oleate, etc. may be used. As bases for suppositories, Witepsol, Macrogol, Tween 61, cocoa butter, lauric acid, glycerogelatin, etc. may be used. Meanwhile, the injection may comprise conventional additives such as solubilizing agents, isotonic agents, suspending agents, emulsifiers, stabilizing agents, and preservatives.

The fourth aspect of the present invention provides a composition for preventing or treating an inflammatory disease, comprising a vegetable oil-derived endo-cannabinoid analogues prepared by the method of the first aspect as an active ingredient.

As used herein, the term “prevention” refers to any activity inhibiting or delaying the occurrence, spread, and recurrence of an inflammatory disease through administering the composition of the present invention, and the term “treatment” refers to any activity that improves or beneficially changes a symptom of the disease by administering the composition of the present invention.

The inflammatory diseases that may be prevented or treated by administering the pharmaceutical composition of the present invention may be acute or chronic inflammatory diseases, such as infectious diseases, allergies, autoimmune diseases, and metabolic diseases. Specifically, the inflammatory diseases may be asthma, arthritis, atopic dermatitis, psoriasis, multiple sclerosis, ssRNA and dsRNA viral infections, sepsis, relapsing polychondritis, scleroderma, eczema, gout, periodontal disease, Behçet's syndrome, edema, vasculitis, Kawasaki disease, diabetic retinopathy, autoimmune pancreatitis, angiitis, glomerulonephritis, acute and chronic bronchitis, Crohn's disease, influenza infections, allergic diseases including allergic asthma, allergic rhinitis, allergic mucositis, urticaria, and anaphylaxis, or myopathies including systemic sclerosis, dermatomyositis, and inclusion body myositis, but is not limited thereto.

In a specific embodiment of the present invention, it was confirmed that the vegetable oil-derived endo-cannabinoid analogues prepared by the method of the present invention significantly reduced the production of nitric oxide, a key marker of inflammatory diseases, in LPS-treated inflammation-induced cells. Furthermore, it was observed that the analogues notably decreased the production of pro-inflammatory cytokines IL-1β, IL-6, and/or TNF-α. Furthermore, it was confirmed that the vegetable oil-derived endo-cannabinoid analogues prepared by the method of the present invention inhibited the production of IL-4, IL-5, and/or IL-13 in a concentration-dependent manner in experimental mouse models in which asthma was induced using ovalbumin.

The fifth aspect of the present invention provides a composition for suppressing pain, comprising a vegetable oil-derived endo-cannabinoid analogues prepared by the method of the first aspect as an active ingredient.

The pain may be inflammatory pain, chronic pain, or acute pain, but is not limited thereto.

In a specific embodiment of the present invention, it was confirmed that the vegetable oil-derived endo-cannabinoid analogues prepared by the method of the present invention reduced pain in formalin test-induced acute inflammatory pain mouse models in a concentration-dependent manner.

The sixth aspect of the present invention provides a method for preventing or treating a cancer or an inflammatory disease, comprising administering the pharmaceutical composition of the fourth or fifth aspect to a subject in need thereof.

As used herein, the term “subject” refers to any animal that may develop cancer or an inflammatory disease, including humans, monkeys, cows, horses, sheep, pigs, chickens, turkeys, quails, cats, dogs, mice, rats, rabbits, or guinea pigs. The pharmaceutical composition of the present invention can effectively prevent or treat the disease by administering the pharmaceutical composition to the subject. The pharmaceutical composition of the present invention may be administered in combination with existing therapeutic agents.

As used herein, the term “administration” refers to the provision of a certain substance to a patient using any appropriate method. The route of administration of the composition of the present invention may be any conventional route, as long as it allows the composition to reach the target tissue. It may be administered intraperitoneally, intravenously, intramuscularly, subcutaneously, intradermally, orally, locally, intranasally, intrapulmonary, and intrarectally, but the route of administration is not limited thereto. In addition, the pharmaceutical composition of the present invention may also be administered by any device capable of transporting the active substance to a target cell. Desirable methods of administration and formulations include intravenous injection, subcutaneous injection, intradermal injection, intramuscular injection, drip injection, etc. The injection may be prepared using aqueous solvents such as saline solution and Ringer's solution, non-aqueous solvents such as vegetable oils, high-grade fatty acid esters (e.g., ethyl oleate), alcohols (e.g., ethanol, benzyl alcohol, propylene glycol, or glycerin), etc. The injection may also comprise pharmaceutical carriers such as stabilizing agents to prevent degradation (e.g., ascorbic acid, sodium hydrosulfite, sodium pyrosulfite, BHA, tocopherol, or EDTA), emulsifiers, buffering agents for pH adjustment, and preservatives to inhibit microbial growth (e.g., phenylmercuric nitrate, thimerosal, benzalkonium chloride, phenol, cresol, or benzyl alcohol).

As used herein, the term “therapeutically effective amount”, which is used in conjunction with an active ingredient, refers to the amount of endo-cannabinoid analogues that is effective in preventing or treating a target disease.

The pharmaceutical composition of the present invention may further comprise, as an active ingredient, a known drug that is used in preventing or treating each disease in addition to the endo-cannabinoid analogues of the present invention, depending on the type of disease to be prevented or treated. For example, when used in preventing or treating a cancer or an inflammatory disease, a known drug may be further included in addition to the compound of the present invention or a pharmaceutically acceptable salt thereof, and the pharmaceutical composition of the present invention may be used in combination with other known treatments for the infection.

In particular, the composition of the present invention is administered in a pharmaceutically effective amount. As used herein, the term “pharmaceutically effective amount” refers to an amount that is sufficient to treat a disease with a reasonable benefit-risk ratio applicable to medical therapy and without causing significant side effects. The effective dosage level may be determined based on factors including the patient's health conditions, type and severity of disease, drug activity, sensitivity to the drug, administration method, administration time, route of administration and elimination rate, treatment duration, and combination or concurrent use of drugs, as well as other well-known factors in the medical field. The composition of the present invention may be administered as a stand-alone treatment or in combination with other treatments, and may be administered sequentially or simultaneously with conventional treatments. The composition of the present invention may be administered in single or multiple doses. It is important to administer the minimum amount that can achieve the maximum effect without side effects, taking all the above-mentioned factors into consideration. This can be readily determined by those skilled in the art.

For example, the administered dose may vary depending on factors such as the route of administration, severity of disease, gender, weight, and age, so the administered dose does not limit the scope of the present invention by any means.

Specifically, the effective amount of the compound in the composition of the present invention may vary depending on the patient's age, gender, and weight, and may be administered in a range of 1 mg to 100 mg, preferably 5 mg to 60 mg, per kg of body weight, daily or every other day, or divided into 1 to 3 doses per day. However, the administered dose may vary depending on factors such as the route of administration, severity of disease, gender, weight, and age, so the administered dose does not limit the scope of the present invention by any means.

The seventh aspect of the present invention provides a feed composition comprising a vegetable oil-derived endo-cannabinoid analogues prepared by the method of the first aspect as an active ingredient.

In a specific embodiment of the present invention, it was confirmed that the vegetable oil-derived endo-cannabinoid analogues prepared by the method of the present invention increased body weight when administered orally to an animal model without inducing toxicity.

The feed composition may include a feed additive. The feed additive of the present invention corresponds to a feed supplement as defined under the Feed Control Act.

As used herein, the term “feed” refers to any natural or artificial diet, meal, or an ingredient of the meal that is intended for or suitable for consumption, ingestion, and digestion by animals.

The type of feed is not particularly limited, and commonly-used feed in the art may be used. Non-limiting examples of the feed include plant-based feed such as grains, root crops, food processing by-products, algae, fibrous materials, pharmaceutical by-products, oils, starches, gourds, or grain by-products; and animal-based feed such as proteins, minerals, fats, mineral-based substances, oils, single-cell proteins, animal plankton, or food. These examples may be used alone or in combination of two or more.

The eighth aspect of the present invention provides a food composition comprising a vegetable oil-derived endo-cannabinoid analogues prepared by the method of the first aspect as an active ingredient.

The food composition of the present invention may be provided in various forms, including food additives or functional foods. Specifically, it may be processed into infusion tea, liquid tea, beverages, fermented milk, cheese, yogurt, juice, probiotics, or health supplements comprising the composition, and it may also be used in various forms of other food additives.

The food composition of the present invention may further include a pharmaceutically acceptable carrier.

There is no particular limitation on the type of food in which the composition may be included. Examples include, but are not limited to, various beverages, gums, tea, vitamin complexes, and health supplements. The food composition may be added with other ingredients that do not interfere with the therapeutic effect on menopausal disorders, and the type of ingredient is not particularly limited. For example, the composition may contain various herbal extracts, foodologically acceptable food supplements, natural carbohydrates, etc. as additional ingredients as in conventional food products.

The food additives are to be added during the preparation of each formulation of the food composition and may be appropriately selected by a person of ordinary skill in the art for use. For example, the composition may include various nutrients, vitamins, minerals (electrolytes), synthetic and natural flavoring agents, coloring agents, fillers, pectic acid and salts thereof, alginic acid and salts thereof, organic acids, protective colloidal thickeners, pH adjusters, stabilizers, preservatives, glycerin, alcohols, and carbonation agents used in carbonated beverages. However, the types of additives are not limited thereto.

The content of the endo-cannabinoid analogues included in the food composition is not particularly limited. However, it may be included in an amount of 0.01 wt % to 100 wt %, specifically 1 wt % to 80 wt %, based on the total weight of the food composition.

In the case of beverages, the analogues may be included at a ratio of 1 g to 30 g per 100 mL, specifically 3 g to 20 g per 100 mL. Additionally, the composition may include additives commonly used in food compositions to enhance aroma, taste, appearance, etc. Examples of the additives include vitamins such as vitamins A, C, D, E, B1, B2, B6, B12, niacin, biotin, folate, and pantothenic acid. Additionally, the additives may include minerals such as zinc (Zn), iron (Fe), calcium (Ca), chromium (Cr), magnesium (Mg), manganese (Mn), and copper (Cu); as well as amino acids such as lysine, tryptophan, cysteine, and valine. Additionally, food additives such as preservatives (e.g., potassium sorbate, sodium benzoate, salicylic acid, sodium dehydroacetate), sterilizers (e.g., bleaching powder, high-strength bleaching powder, sodium hypochlorite), antioxidants (e.g., butylated hydroxyanisole (BHA), butylated hydroxytoluene (BHT)), coloring agents (e.g., tar dyes), color formers (e.g., sodium nitrite, sodium ascorbate), bleaching agents (e.g., sodium sulfite), seasonings (e.g., monosodium glutamate (MSG)), sweeteners (e.g., dulcin, cyclamate, saccharin, sodium), flavoring agents (e.g., vanillin, lactones), leavening agents (e.g., alum, D-potassium hydrogen tartrate), fortifiers, emulsifiers, thickeners (stabilizers), coating agents, gum bases, anti-foaming agents, solvents, and modifiers may be added. The additives are selected based on the type of food and are used in an appropriate amount.

The food composition of the present invention can be prepared using conventional methods in the art, and raw materials and ingredients conventionally added in the art during preparation may also be included. Additionally, unlike conventional pharmaceuticals, the composition is derived from food-based ingredients, offering the advantage of minimizing potential side effects associated with long-term drug administration. Furthermore, it may exhibit excellent portability.

The ninth aspect of the present invention provides a cosmetic composition comprising a vegetable oil-derived endo-cannabinoid analogues prepared by the method of the first aspect as an active ingredient.

In the present invention, the cosmetic composition may be prepared into a formulation selected from the group consisting of solutions, topical ointments, creams, foams, nourishing toners, softening toners, masks, softeners, emulsions, makeup bases, essences, soaps, liquid cleansers, bath agents, sunscreen creams, sun oils, suspensions, emulsions, pastes, gels, lotions, powders, soaps, surfactant-containing cleansers, oils, powder foundations, emulsion foundations, wax foundations, patches, and sprays, but is not limited thereto.

The cosmetic composition of the present invention may further include one or more cosmetically acceptable carriers commonly used in general skin cosmetics. Conventional ingredients, such as oils, water, surfactants, moisturizers, lower alcohols, thickeners, chelating agents, colorants, preservatives, and fragrances, may be appropriately blended, but the composition is not limited thereto.

The cosmetically acceptable carriers included in the cosmetic composition of the present invention may vary depending on the formulation of the cosmetic composition.

When the formulation of the present invention is an ointment, paste, cream, or gel, the carrier components may include animal oils, vegetable oils, waxes, paraffin, starch, tragacanth, cellulose derivatives, polyethylene glycol, silicones, bentonite, silica, talc, and zinc oxide, but are not limited thereto. These examples may be used alone or in combination of two or more.

When the formulation of the present invention is a powder or spray, the carrier components may include lactose, talc, silica, aluminum hydroxide, calcium silicate, and polyamide powder. In particular, when formulated as a spray, the composition may additionally include propellants such as chlorofluorohydrocarbons, propane/butane, or dimethyl ether, but is not limited thereto. These examples may be used alone or in combination of two or more.

When the formulation of the present invention is a solution or emulsion, solvents, solubilizers, emulsifiers, etc. may be used as carrier components. Examples include water, ethanol, isopropanol, ethyl carbonate, ethyl acetate, benzyl alcohol, benzyl benzoate, propylene glycol, 1,3-butylene glycol, and oils. In particular, cottonseed oil, peanut oil, corn germ oil, olive oil, castor oil, sesame oil, glycerol fatty acid esters, polyethylene glycol, or sorbitan fatty acid esters may be used, but the composition is not limited thereto. These examples may be used alone or in combination of two or more.

When the formulation of the present invention is a suspension, the carrier components may include liquid diluents such as water, ethanol, or propylene glycol; suspending agents such as ethoxylated isostearyl alcohol, polyoxyethylene sorbitol esters, and polyoxyethylene sorbitan esters; microcrystalline cellulose; aluminum metahydroxide; bentonite; agar; or tragacanth, but are not limited thereto. These examples may be used alone or in combination of two or more.

When the formulation of the present invention is a surfactant-containing cleanser, the carrier components may include fatty alcohol sulfates, fatty alcohol ether sulfates, sulfosuccinate monoesters, isethionates, imidazolinium derivatives, methyl taurates, sarcosinates, fatty acid amide ether sulfates, alkylamidobetaines, aliphatic alcohols, fatty acid glycerides, fatty acid diethanolamides, vegetable oils, lanolin derivatives, or ethoxylated glycerol fatty acid esters, but are not limited thereto. These examples may be used alone or in combination of two or more.

The cosmetic composition of the present invention may include other ingredients that are commonly used in cosmetics, in addition to the essential components, as necessary. Specifically, possible additive ingredients include oils, moisturizers, emollients, surfactants, organic and inorganic pigments, organic powders, UV absorbers, preservatives, disinfectants, antioxidants, plant extracts, pH adjusters, alcohols, colorants, fragrances, blood circulation promoters, cooling agents, antiperspirants, and purified water, but are not limited thereto.

### MODE FOR CARRYING OUT THE INVENTION

The present disclosure will be described in detail by way of Examples. The Examples are provided to specifically explain the present invention, and the scope of the present invention is not limited by the Examples.

### Example 1: Preparation of Endo-Cannabinoid Analogues Through Enzymatic Conversion of Triglycerides

### 1-1. Preparation of Endo-Cannabinoid Analogues (SFO-AP) Using Sunflower Oil and 3-Amino-1-Propanol

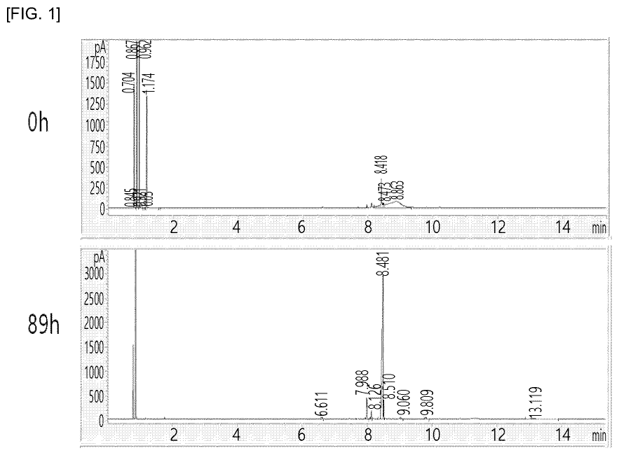

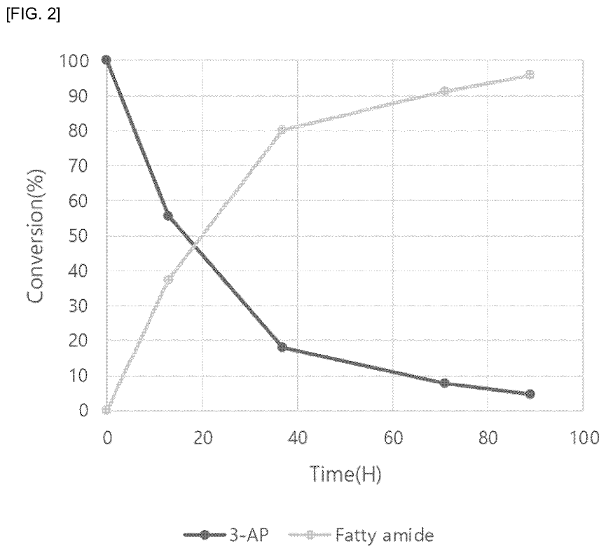

1 M sunflower oil (SFO) and 3 M 3-amino-1-propanol (AP) were added into a double-jacketed reactor maintained at 55° C. and mixed. Subsequently, 1% (w/v) of immobilized lipase enzyme was added, and the amidation reaction was carried out for 89 hours. Samples were taken at regular time intervals during the reaction, and the samples were analyzed using gas chromatography (GC) to analyze the conversion process (FIG. 1). The GC system used was a 7890a Gas Chromatography/Flame Ionization Detector (GC/FID) system (Agilent Technologies, Santa Clara, CA, USA). The column used was an HP-5 column (30 m length, 0.320 mm diameter, 0.25 μm film thickness). 50 μL of the enzyme reaction solution was mixed with 950 μL of chloroform, and a final volume of 1 μL was used for the GC analysis. The temperature of the GC oven was increased from 80° C. to 300° C. at a rate of 30° C./min. As a carrier gas, helium was maintained at a flow rate of 30 mL/min, while oxygen and hydrogen gases were maintained at flow rates of 400 mL/min and 60 mL/min, respectively. The FID detector temperature was set to 270° C. The fatty acid composition of SFO consists of 59% linoleic acid, 30% oleic acid, 6% stearic acid, and 5% palmitic acid. The GC peaks observed after the AP amidation reaction showed that, excluding palmitic acid AP amide (retention time (RT) of 7.9 minutes), the other three fatty acid amides-stearic acid AP amide, oleic acid AP amide, and linoleic acid AP amide-having the same carbon chain length were detected at the same peak at an RT of 8.5 minutes (FIG. 1). The final AP amide conversion of SFO was 96.3% after 89 hours (FIG. 2). The characteristics and composition of the resulting fatty acid AP amides are presented in Table 1.

TABLE 1

| | | | Molecular | |
|---|---|---|---|---|
| Fatty acid AP | | Chemical | Weight | Content |
| amide | Structure | Formula | (Da) | (%) |
| Linoleamide-AP | 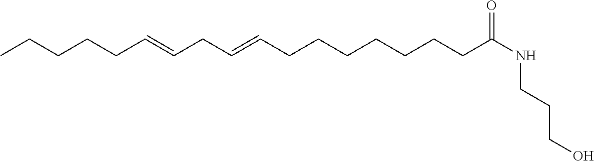 [SMILES: CCCCC/C=C/C/C=C/CCCCCCCC(=O)NCCCO] | C₂₁H₃₉NO₂ | 337.55 | ~59 |
| Oleamide-AP | 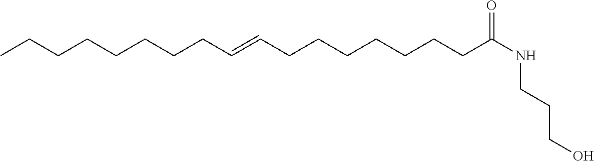 [SMILES: CCCCCCCC/C=C/CCCCCCCC(=O)NCCCO] | C₂₁H₄₁NO₂ | 339.56 | ~30 |
| Stearamide-AP | 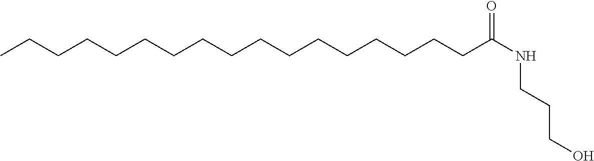 [SMILES: CCCCCCCCCCCCCCCCCC(=O)NCCCO] | C₂₁H₄₃NO₂ | 341.58 | ~6 |
| Palmitamide-AP | 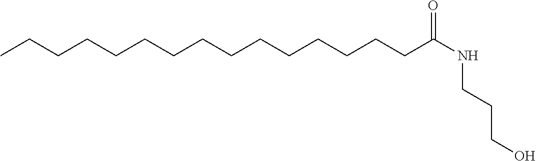 [SMILES: CCCCCCCCCCCCCCCC(=O)NCCCO] | C₁₉H₃₉NO₂ | 313.53 | ~5 |

### 1-2. Preparation of Endo-Cannabinoid Analogues (SFO-EA) Using Sunflower Oil and Ethanolamine

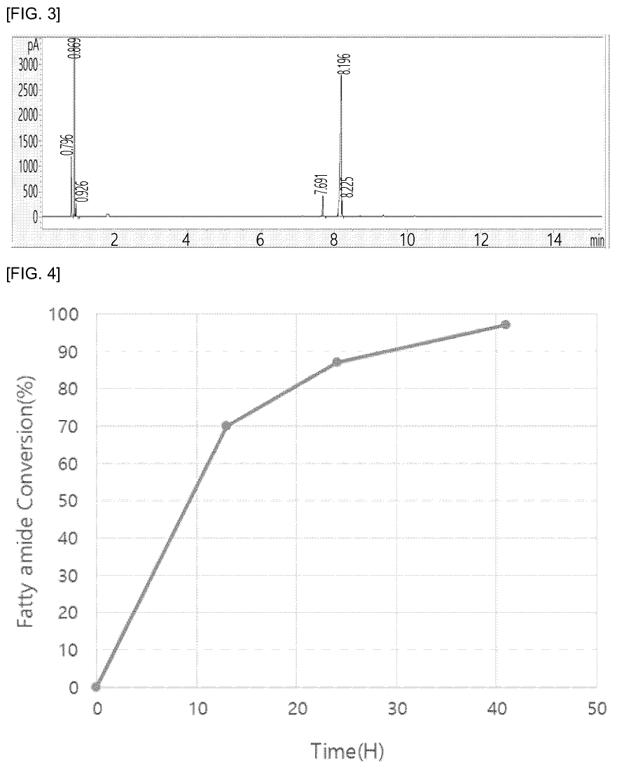

An amidation reaction was carried out and analyzed using a method similar to that of Example 1-1, except that ethanolamine (EA) was used instead of AP, and the reaction was conducted for 42 hours. The GC peaks observed after the EA amidation reaction showed that, excluding palmitic acid EA amide (RT 7.9 minutes), the other three fatty acid amides-stearic acid EAamide, oleic acid EAamide, and linoleic acid EAamide-having the same carbon chain length, were detected at the same peak at RT 8.2 minutes (FIG. 3). The final EA amide conversion of SFO was 97% after 41 hours (FIG. 4). The characteristics and composition of the resulting fatty acid EA amides are presented in Table 2.

TABLE 2

| | | | Molecular | |
|---|---|---|---|---|
| Fatty acid EA | | Chemical | Weight | Content |
| amide | Structure | Formula | (Da) | (%) |
| Linoleamide-EA | 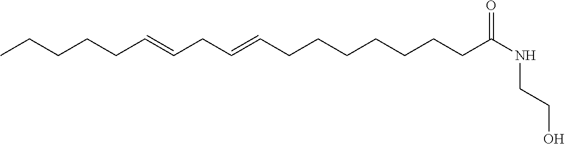 [SMILES: CCCCC/C=C/C/C=C/CCCCCCCC(=O)NCCO] | C₂₀H₃₇NO₂ | 323.52 | ~59 |
| Oleamide-EA | 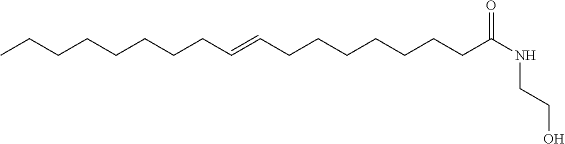 [SMILES: CCCCCCCC/C=C/CCCCCCCC(=O)NCCO] | C₂₀H₃₉NO₂ | 325.54 | ~30 |
| Stearamide-EA | 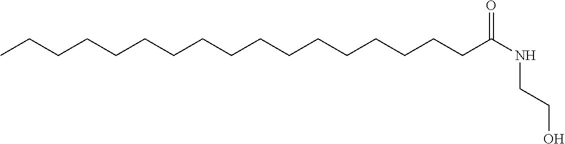 [SMILES: CCCCCCCCCCCCCCCCCC(=O)NCCO] | C₂₀H₄₁NO₂ | 327.55 | ~6 |
| Palmitamide-EA | 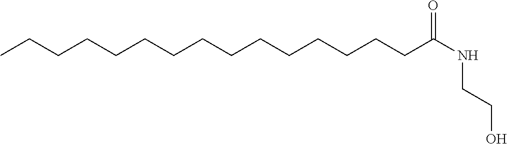 [SMILES: CCCCCCCCCCCCCCCC(=O)NCCO] | C₁₈H₃₇NO₂ | 299.50 | ~5 |

### 1-3. Preparation of Endo-Cannabinoid Analogues (SFO-MIPA) Using Sunflower Oil and Monoisopropanolamine

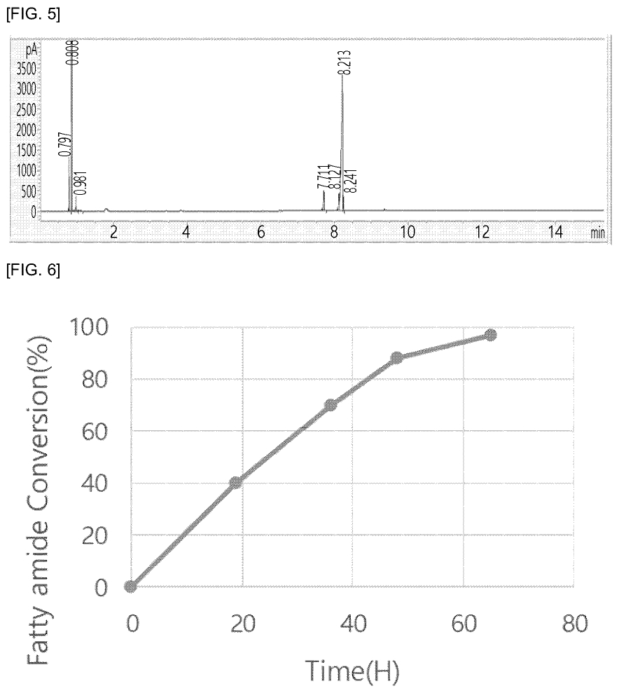

An amidation reaction was carried out and analyzed using a method similar to that of Example 1-1, except that monoisopropanolamine (M IPA) was used instead of AP, and the reaction was conducted for 66 hours. The GC peaks observed after the MIPA amidation reaction showed that, excluding palmitic acid MIPA amide (RT 7.7 minutes), the other three fatty acid amides-stearic acid MIPA amide, oleic acid MIPA amide, and linoleic acid MIPA amide-having the same carbon chain length, were detected at the same peak at RT 8.2 minutes (FIG. 5). The final MIPA amide conversion of SFO was 97.5% after 65 hours (FIG. 6). The characteristics and composition of the resulting fatty acid MIPA amides are presented in Table 3.

TABLE 3

| | | | Molecular | |
|---|---|---|---|---|
| Fatty acid | | Chemical | Weight | Content |
| MIPA amide | Structure | Formula | (Da) | (%) |
| Linoleamide- MIPA | 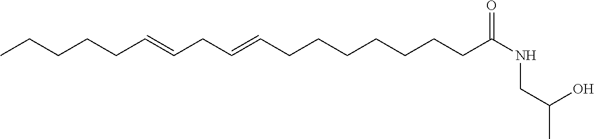 [SMILES: CCCCC/C=C/C/C=C/CCCCCCCC(=O)NCC(C)O] | C₂₁H₃₉NO₂ | 337.55 | ~59 |
| Oleamide- MIPA | 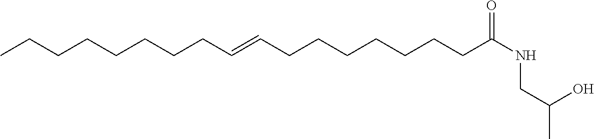 [SMILES: CCCCCCCC/C=C/CCCCCCCC(=O)NCC(C)O] | C₂₁H₄₁NO₂ | 339.56 | ~30 |
| Stearamide- MIPA | 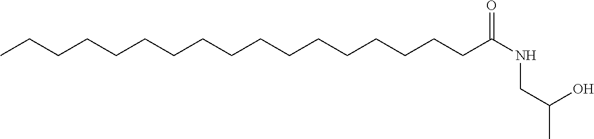 [SMILES: CCCCCCCCCCCCCCCCCC(=O)NCC(C)O] | C₂₁H₄₃NO₂ | 341.58 | ~6 |
| Palmitamide- MIPA | 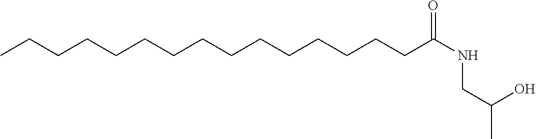 [SMILES: CCCCCCCCCCCCCCCC(=O)NCC(C)O] | C₁₉H₃₉NO₂ | 313.53 | ~5 |

### 1-4. Preparation of Endo-Cannabinoid Analogues (SFO-AMP) Using Sunflower Oil and 2-Amino-2-Methyl-1-Propanol

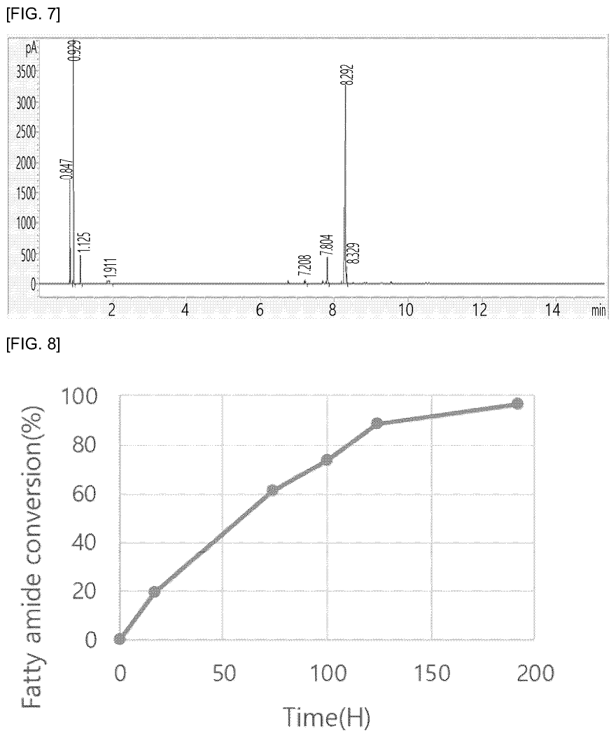

An amidation reaction was carried out and analyzed using a method similar to that of Example 1-1, except that 2-amino-2-methyl-1-propanol (AMP) was used instead of AP, and the reaction was conducted for 150 hours. The GC peaks observed after the AMP amidation reaction showed that, excluding palmitic acid AMP amide (RT 7.8 minutes), the other three fatty acid amides-stearic acid AMP amide, oleic acid AMP amide, and linoleic acid AMP amide-having the same carbon chain length, were detected at the same peak at RT 8.29 minutes (FIG. 7). The final AMP amide conversion of SFO was 96.5% after 192 hours (FIG. 8). The characteristics and composition of the resulting fatty acid AMP amides are presented in Table 4.

TABLE 4

| | | | Molecular | |
|---|---|---|---|---|
| Fatty acid AMP | | Chemical | Weight | Content |
| amide | Structure | Formula | (Da) | (%) |
| Linoleamide-AMP | 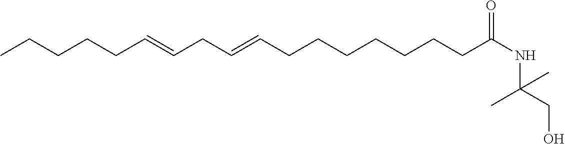 [SMILES: CCCCC/C=C/C/C=C/CCCCCCCC(=O)NC(C)(C)CO] | C₂₂H₄₁NO₂ | 351.57 | ~59 |
| Oleamide-AMP | 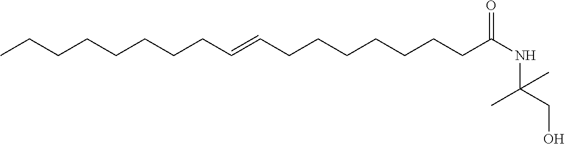 [SMILES: CCCCCCCC/C=C/CCCCCCCC(=O)NC(C)(C)CO] | C₂₂H₄₃NO₂ | 353.59 | ~30 |
| Stearamide-AMP | 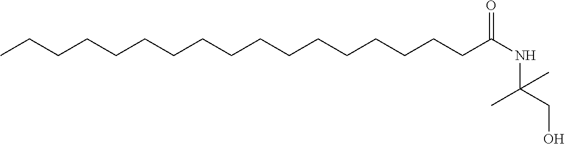 [SMILES: CCCCCCCCCCCCCCCCCC(=O)NC(C)(C)CO] | C₂₂H₄₅NO₂ | 355.35 | ~6 |
| Palmitamide-AMP | 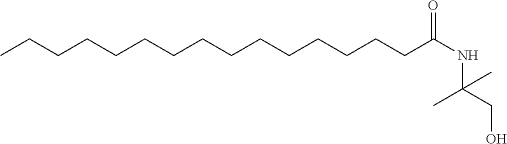 [SMILES: CCCCCCCCCCCCCCCC(=O)NC(C)(C)CO] | C₂₀H₄₁NO₂ | 327.55 | ~5 |

### 1-5. Preparation of Endo-Cannabinoid Analogues (OLO-AP) Using Olive Oil and 3-Amino-1-Propanol

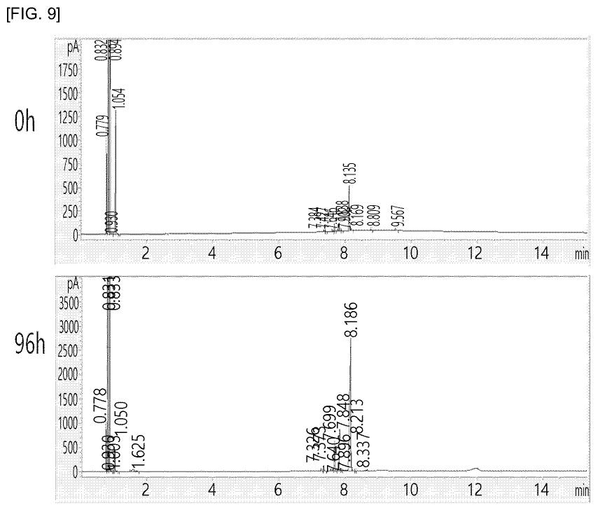

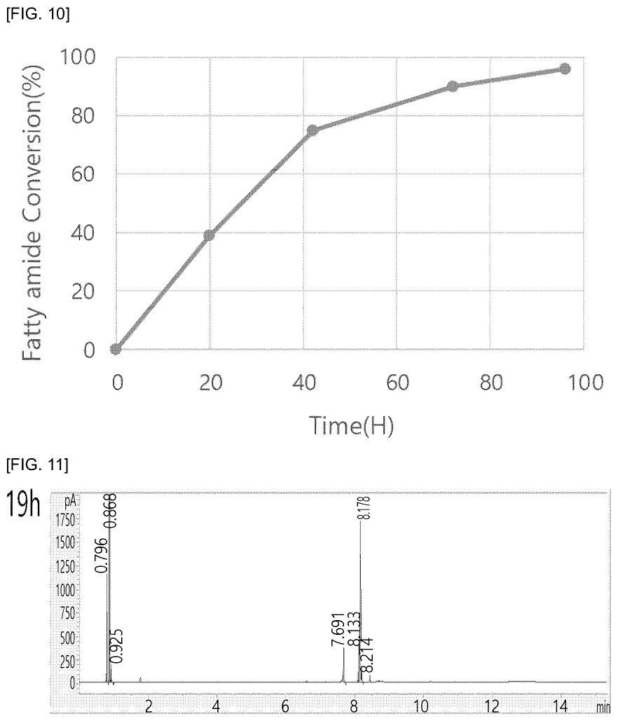

An amidation reaction was carried out and analyzed using a method similar to that of Example 1-1, except that olive oil (OLO) was used instead of SFO, and the reaction was conducted for 96 hours. The fatty acid composition of olive oil consists of 71% oleic acid, 13% palmitic acid, 12% linoleic acid, and 4% stearic acid. The GC peaks observed after the AP amidation reaction showed that, excluding palmitic acid AP amide (RT 7.7 minutes), the other three fatty acid amides-stearic acid AP amide, oleic acid AP amide, and linoleic acid AP amide-having the same carbon chain length, were detected at the same peak at RT 8.2 minutes (FIG. 9). The final AP amide conversion of OLO was 95.3% after 96 hours (FIG. 10). The characteristics and composition of the resulting fatty acid AP amides are presented in Table 5.

TABLE 5

| | | | Molecular | |
|---|---|---|---|---|
| Fatty acid AP | | Chemical | Weight | Content |
| amide | Structure | Formula | (Da) | (%) |
| Oleamide-AP | 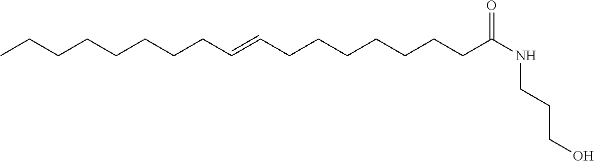 [SMILES: CCCCCCCC/C=C/CCCCCCCC(=O)NCCCO] | C₂₁H₄₁NO₂ | 339.56 | ~71 |
| Palmitamide-AP | 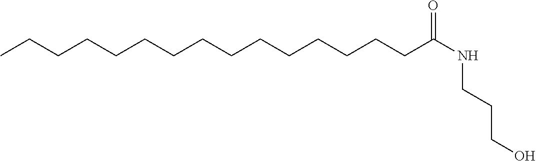 [SMILES: CCCCCCCCCCCCCCCC(=O)NCCCO] | C₁₉H₃₉NO₂ | 313.53 | ~13 |
| Linoleamide-AP | 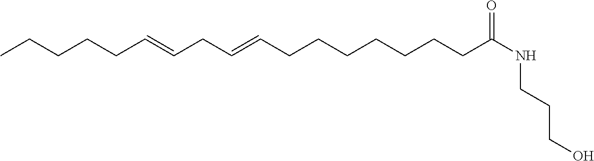 [SMILES: CCCCC/C=C/C/C=C/CCCCCCCC(=O)NCCCO] | C₂₁H₃₉NO₂ | 337.55 | ~12 |
| Stearamide-AP | 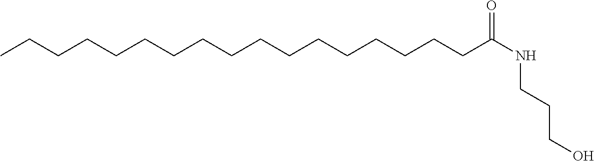 [SMILES: CCCCCCCCCCCCCCCCCC(=O)NCCCO] | C₂₁H₄₃NO₂ | 341.58 | ~4 |

### 1-6. Preparation of Endo-Cannabinoid Analogues (OLO-EA) Using Olive Oil and Ethanolamine

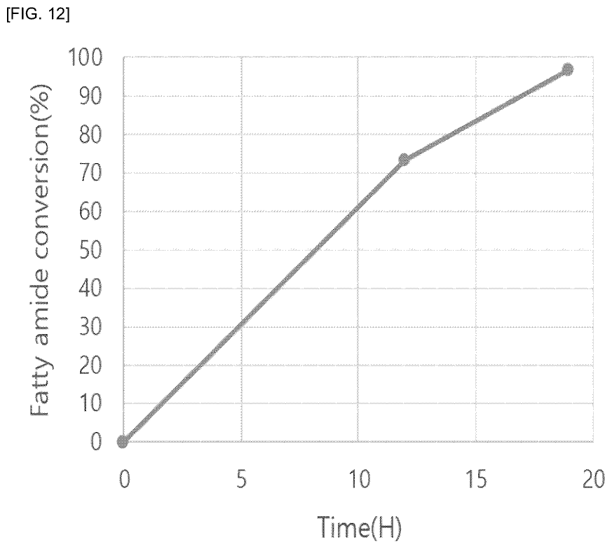

An amidation reaction was carried out and analyzed using a method similar to that of Example 1-2, except that OLO was used instead of SFO, and the reaction was conducted for 17 hours. The GC peaks observed after the EA amidation reaction showed that, excluding palmitic acid EA amide (RT 7.69 minutes), the other three fatty acid amides-stearic acid EA amide, oleic acid EA amide, and linoleic acid EA amide-having the same carbon chain length, were detected at the same peak at RT 8.18 minutes (FIG. 11). The final EA amide conversion of OLO was 94.7% after 17 hours (FIG. 12). The characteristics and composition of the resulting fatty acid EA amides are presented in Table 6.

TABLE 6

| | | | Molecular | |
|---|---|---|---|---|
| Fatty acid EA | | Chemical | Weight | Content |
| amide | Structure | Formula | (Da) | (%) |
| Oleamide-EA | 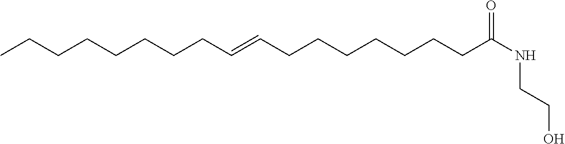 [SMILES: CCCCCCCC/C=C/CCCCCCCC(=O)NCCO] | C₂₀H₃₉NO₂ | 325.54 | ~71 |
| Palmitamide-EA | 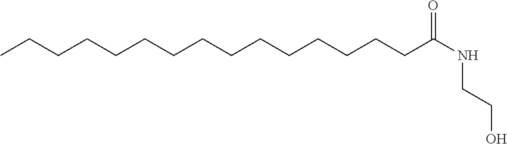 [SMILES: CCCCCCCCCCCCCCCC(=O)NCCO] | C₁₈H₃₇NO₂ | 299.50 | ~13 |
| Linoleamide-EA | 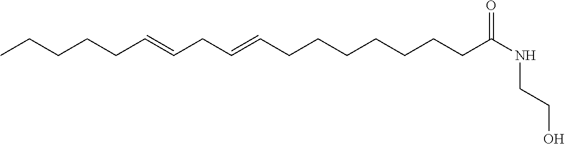 [SMILES: CCCCC/C=C/C/C=C/CCCCCCCC(=O)NCCO] | C₂₀H₃₇NO₂ | 323.52 | ~12 |
| Stearamide-EA | 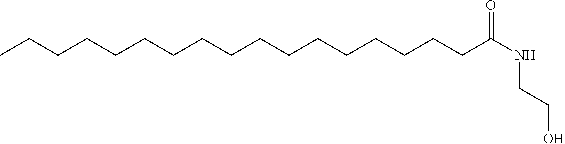 [SMILES: CCCCCCCCCCCCCCCCCC(=O)NCCO] | C₂₀H₄₁NO₂ | 327.55 | ~4 |

### 1-7. Preparation of Endo-Cannabinoid Analogues (CNO-AP) Using Coconut Oil and 3-Amino-1-Propanol

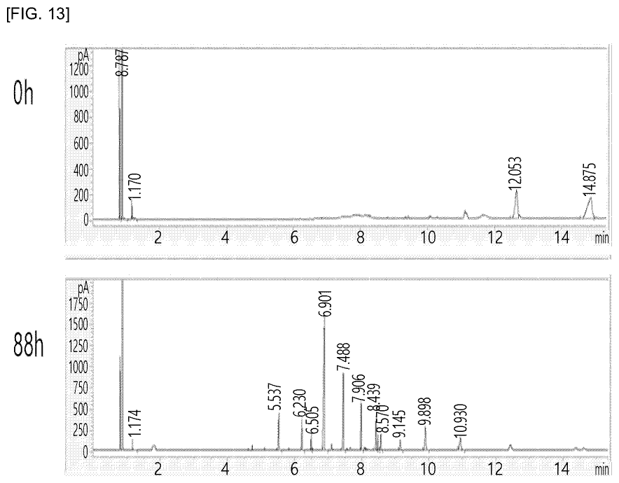

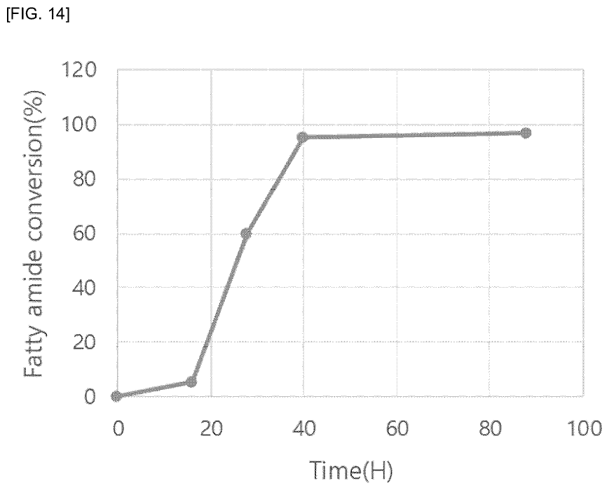

An amidation reaction was carried out and analyzed using a method similar to that of Example 1-1, except that coconut oil (CNO) was used instead of SFO, and the reaction was conducted for 96 hours. The fatty acid composition of CNO consists of 47% lauric acid, 18% myristic acid, 9% palmitic acid, 6% capric acid, 6% oleic acid, 3% stearic acid, and 2% linoleic acid. The GC peaks observed after the AP amidation reaction were identified as lauric acid AP amide (RT 6.9 minutes), myristic acid AP amide (RT 7.46 minutes), and palmitic acid AP amide (RT 7.8 minutes) (FIG. 13). The final AP amide conversion of CNO was 96.5% after 88 hours (FIG. 14). The characteristics and composition of the resulting fatty acid AP amides are presented in Table 7.

TABLE 7

| | | | Molecular | |
|---|---|---|---|---|
| Fatty acid AP | | Chemical | Weight | Content |
| amide | Structure | Formula | (Da) | (%) |
| Lauramide-AP | 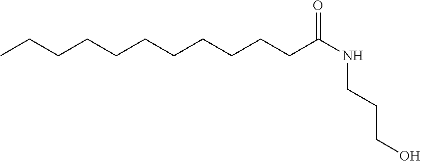 [SMILES: CCCCCCCCCCCC(=O)NCCCO] | C₁₅H₃₁NO₂ | 285.47 | ~47 |
| Myristamide-AP | 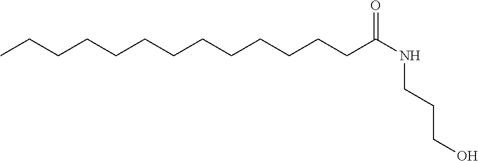 [SMILES: CCCCCCCCCCCCCC(=O)NCCCO] | C₁₇H₃₅NO₂ | 285.47 | ~18 |
| Palmitamide-AP | 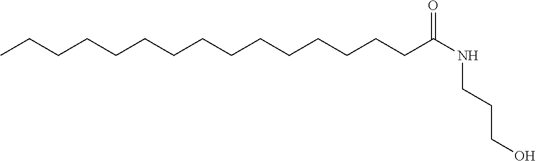 [SMILES: CCCCCCCCCCCCCCCC(=O)NCCCO] | C₁₉H₃₉NO₂ | 313.53 | ~9 |
| Capramide-AP | 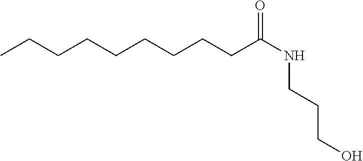 [SMILES: CCCCCCCCCC(=O)NCCCO] | C₁₃H₂₇NO₂ | 229.36 | ~6 |

### 1-8. Preparation of Endo-Cannabinoid Analogues (CNO-EA) Using Coconut Oil and Ethanolamine

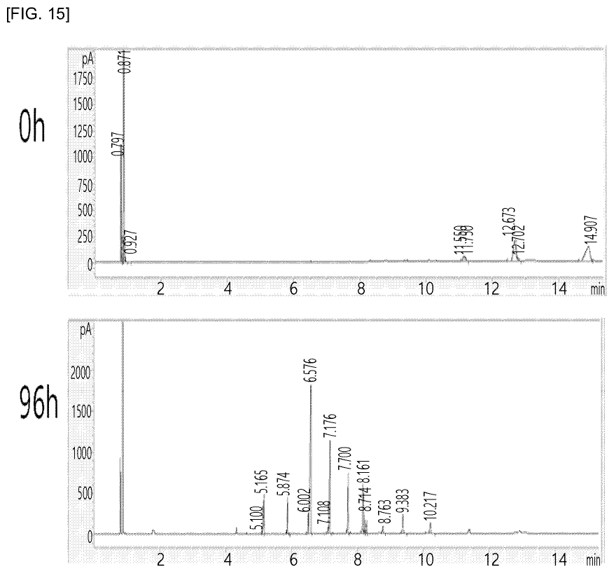

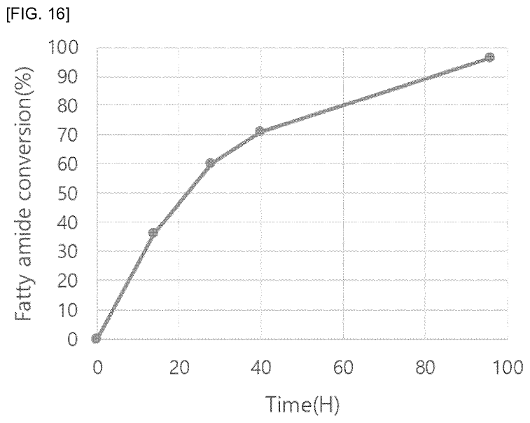

An amidation reaction was carried out and analyzed using a method similar to that of Example 1-7, except that EA was used instead of AP, and the reaction was conducted for 96 hours. The GC peaks observed after the EA amidation reaction were identified as lauric acid EA amide (RT 6.57 minutes), myristic acid EA amide (RT 7.15 minutes), and palmitic acid EA amide (RT 7.7 minutes) (FIG. 15). The final EA amide conversion of OLO was 96.4% after 96 hours (FIG. 16). The characteristics and composition of the resulting fatty acid EA amides are presented in Table 8.

TABLE 8

| | | | Molecular | |
|---|---|---|---|---|
| Fatty acid EA | | Chemical | Weight | Content |
| amide | Structure | Formula | (Da) | (%) |
| Lauramide-EA | 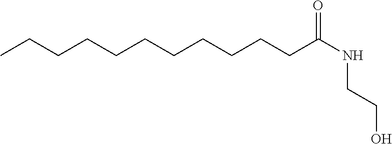 [SMILES: CCCCCCCCCCCC(=O)NCCO] | C₁₄H₂₉NO₂ | 243.39 | ~47 |
| Myristamide-EA | 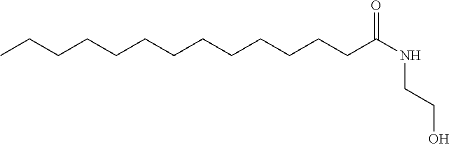 [SMILES: CCCCCCCCCCCCCC(=O)NCCO] | C₁₆H₃₃NO₂ | 271.44 | ~18 |
| Palmitamide-EA | 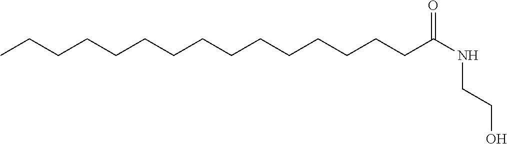 [SMILES: CCCCCCCCCCCCCCCC(=O)NCCO] | C₁₈H₃₇NO₂ | 299.50 | ~9 |
| Capramide-EA | 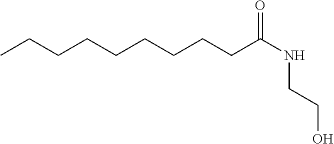 | C₁₂H₂₅NO₂ | 215.34 | ~6 |

### 1-9. Preparation of Endo-Cannabinoid Analogues (SBO-AP) Using Soybean Oil and 3-Amino-1-Propanol

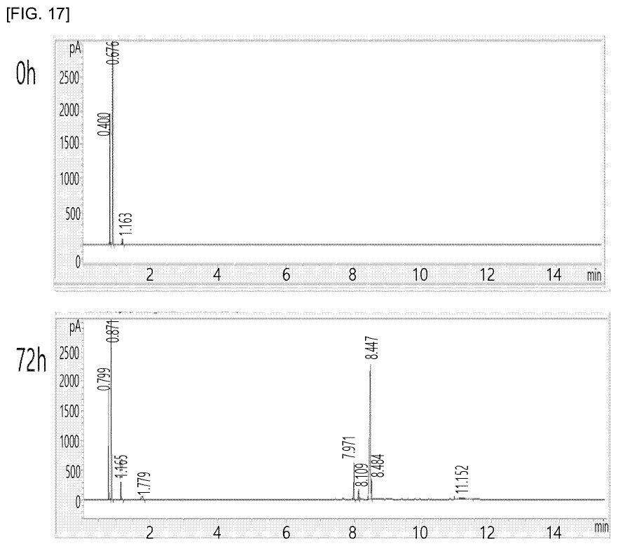

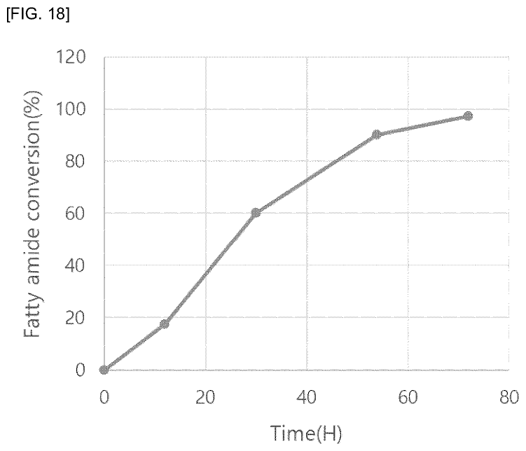

An amidation reaction was carried out and analyzed using a method similar to that of Example 1-1, except that soybean oil (SBO) was used instead of SFO, and the reaction was conducted for 96 hours. The fatty acid composition of SBO consists of 54% linoleic acid, 24% oleic acid, 11% palmitic acid, 7% linolenic acid, and 4% stearic acid. The GC peaks observed after the AP amidation reaction were identified as palmitic acid AP amide at RT 7.97 minutes, the three fatty acid amides-stearic acid AP amide, oleic acid AP amide, and linoleic acid AP amide-having the same carbon chain length at the same peak at RT 8.45 minutes, and linolenic acid AP amide at RT 8.48 minutes (FIG. 17). The final AP amide conversion of SBO was 96.9% after 72 hours (FIG. 18). The characteristics and composition of the resulting fatty acid AP amides are presented in Table 9.

TABLE 9

| | | | Molecular | |
|---|---|---|---|---|
| Fatty acid AP | | Chemical | Weight | Content |
| amide | Structure | Formula | (Da) | (%) |
| Linoleamide- AP | 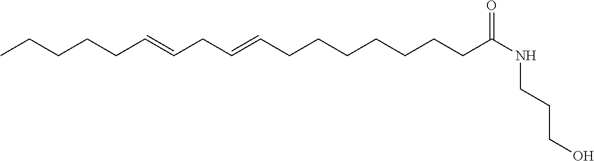 [SMILES: CCCCC/C=C/C/C=C/CCCCCCCC(=O)NCCCO] | C₂₁H₃₉NO₂ | 337.55 | ~54 |
| Oleamide- AP | 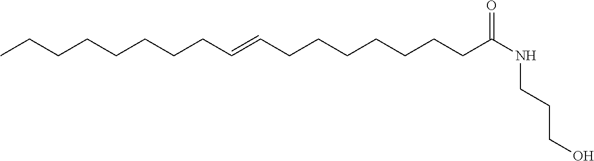 [SMILES: CCCCCCCC/C=C/CCCCCCCC(=O)NCCCO] | C₂₁H₄₁NO₂ | 339.56 | ~24 |
| Palmitamide- AP | 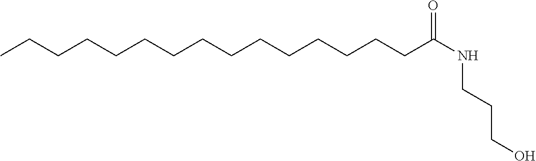 [SMILES: CCCCCCCCCCCCCCCC(=O)NCCCO] | C₁₉H₃₉NO₂ | 313.53 | ~11 |
| Linolenamide- AP | 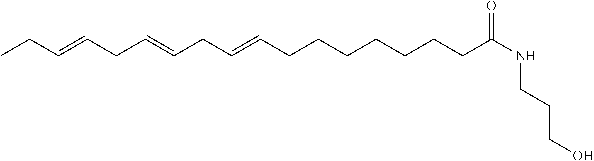 [SMILES: CC/C=C/C/C=C/C/C=C/CCCCCCCC(=O)NCCCO] | C₂₁H₃₇NO₂ | 335.53 | ~7 |
| Stearamide- AP | 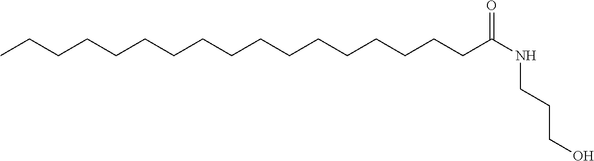 [SMILES: CCCCCCCCCCCCCCCCCC(=O)NCCCO] | C₂₁H₄₃NO₂ | 341.58 | ~4 |

### 1-10. Preparation of Endo-Cannabinoid Analogues (SBO-EA) Using Soybean Oil and Ethanolamine

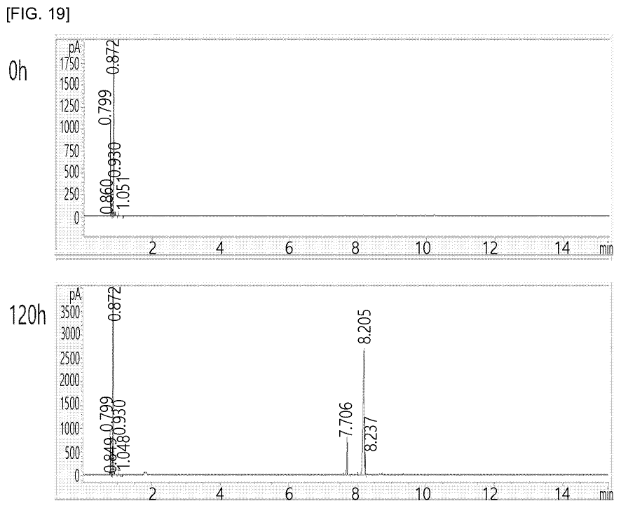

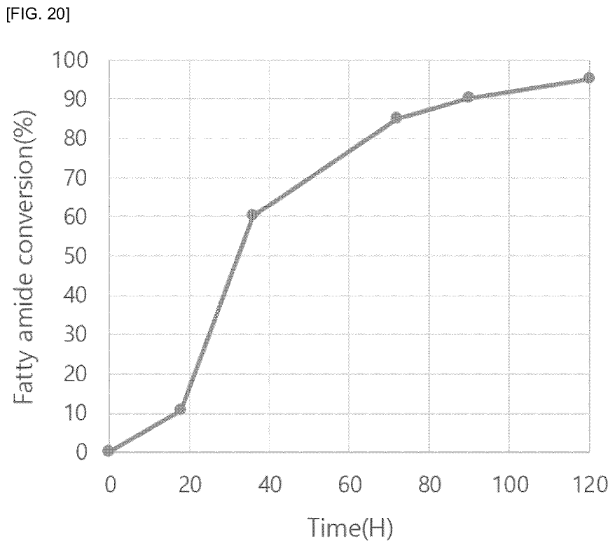

An amidation reaction was carried out and analyzed using a method similar to that of Example 1-9, except that EA was used instead of AP, and the reaction was conducted for 96 hours. The GC peaks observed after the EA amidation reaction were identified as palmitic acid EA amide at RT 7.7 minutes, the three fatty acid amides-stearic acid EA amide, oleic acid EA amide, and linoleic acid EA amide-having the same carbon chain length at the same peak at RT 8.2 minutes, and linolenic acid EA amide at RT 8.24 minutes (FIG. 19). The final EA amide conversion of SBO was 95.2% after 120 hours (FIG. 20). The characteristics and composition of the resulting fatty acid AP amides are presented in Table 10.

TABLE 10

| | | | Molecular | |
|---|---|---|---|---|
| Fatty acid EA | | Chemical | Weight | Content |
| amide | Structure | Formula | (Da) | (%) |
| Linoleamide-EA | 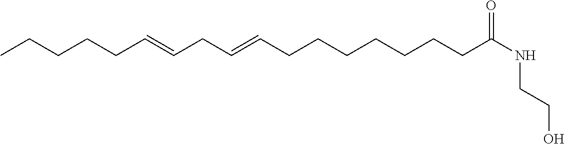 [SMILES: CCCCC/C=C/C/C=C/CCCCCCCC(=O)NCCO] | C₂₀H₃₇NO₂ | 323.52 | ~54 |
| Oleamide-EA | 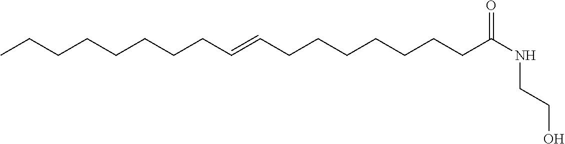 [SMILES: CCCCCCCC/C=C/CCCCCCCC(=O)NCCO] | C₂₀H₃₉NO₂ | 325.54 | ~24 |
| Palmitamide-EA | 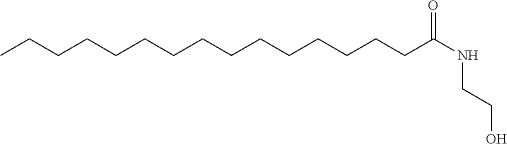 [SMILES: CCCCCCCCCCCCCCCC(=O)NCCO] | C₁₈H₃₇NO₂ | 299.50 | ~11 |
| Linolenamide-EA | 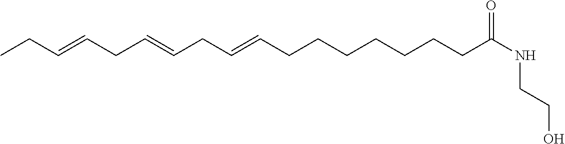 [SMILES: CC/C=C/C/C=C/C/C=C/CCCCCCCC(=O)NCCO] | C₂₀H₃₅NO₂ | 321.50 | ~7 |
| Stearamide-EA | 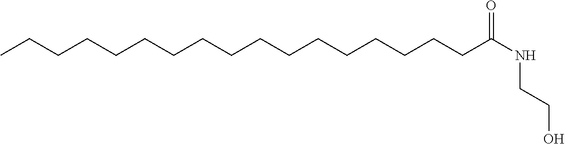 [SMILES: CCCCCCCCCCCCCCCCCC(=O)NCCO] | C₂₀H₄₁NO₂ | 327.55 | ~4 |

### 1-11. Preparation of Endo-Cannabinoid Analogues (GSO-AP) Using Grapeseed Oil and 3-Amino-1-Propanol

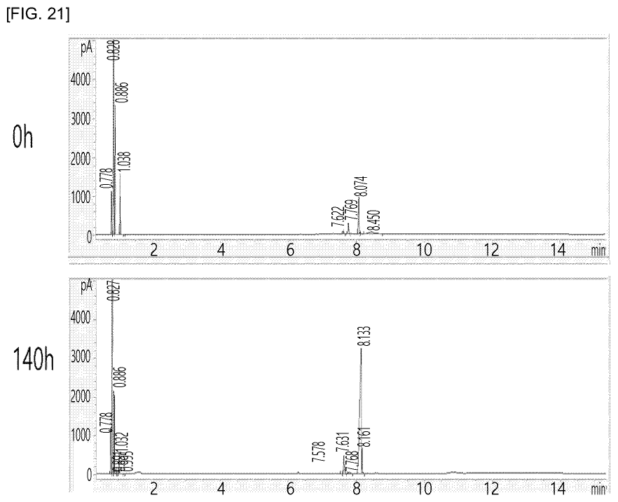

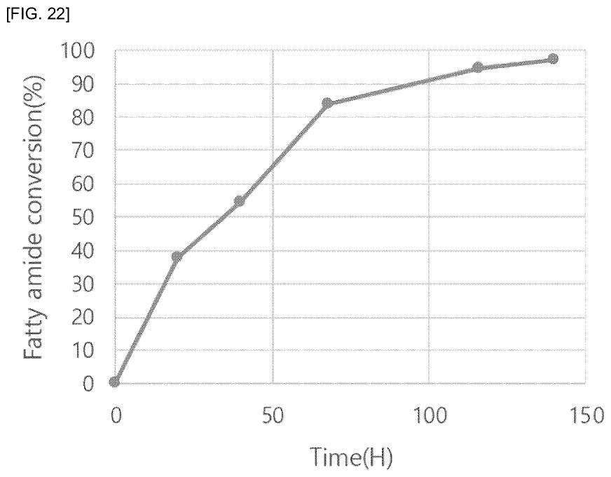

An amidation reaction was carried out and analyzed using a method similar to that of Example 1-1, except that grapeseed oil (GSO) was used instead of SFO, and the reaction was conducted for 96 hours. The fatty acid composition of GSO consists of 73% linoleic acid, 15% oleic acid, 8% palmitic acid, 4% stearic acid, etc. The GC peaks observed after the AP amidation reaction were identified as palmitic acid AP amide at RT 7.63 minutes, the three fatty acid amides-stearic acid AP amide, oleic acid AP amide, and linoleic acid AP amide-having the same carbon chain length at RT 8.13 minutes, and linolenic acid AP amide at RT 8.16 minutes (FIG. 21). The final AP amide conversion of GSO was 97% after 140 hours (FIG. 22). The characteristics and composition of the resulting fatty acid AP amides are presented in Table 11.

TABLE 11

| | | | Molecular | |
|---|---|---|---|---|
| Fatty acid AP | | Chemical | Weight | Content |
| amide | Structure | Formula | (Da) | (%) |
| Linoleamide-AP | 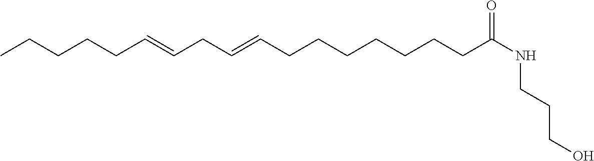 [SMILES: CCCCC/C=C/C/C=C/CCCCCCCC(=O)NCCCO] | C₂₁H₃₉NO₂ | 337.55 | ~73 |
| Oleamide-AP | 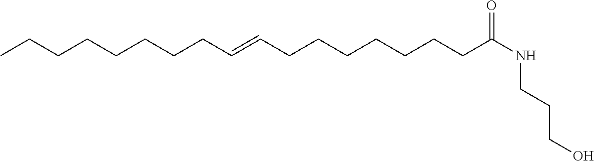 [SMILES: CCCCCCCC/C=C/CCCCCCCC(=O)NCCCO] | C₂₁H₄₁NO₂ | 339.56 | ~15 |
| Palmitamide-AP | 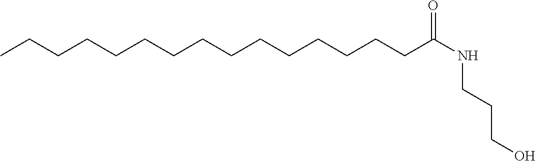 [SMILES: CCCCCCCCCCCCCCCC(=O)NCCCO] | C₁₉H₃₉NO₂ | 313.53 | ~8 |
| Stearamide-AP | 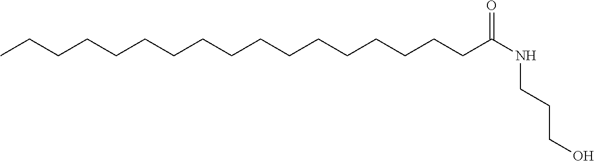 [SMILES: CCCCCCCCCCCCCCCCCC(=O)NCCCO] | C₂₁H₄₃NO₂ | 341.58 | ~4 |

### 1-12. Preparation of Endo-Cannabinoid Analogues (EPO-AP) Using Evening Primrose Oil and 3-Amino-C-Propanol

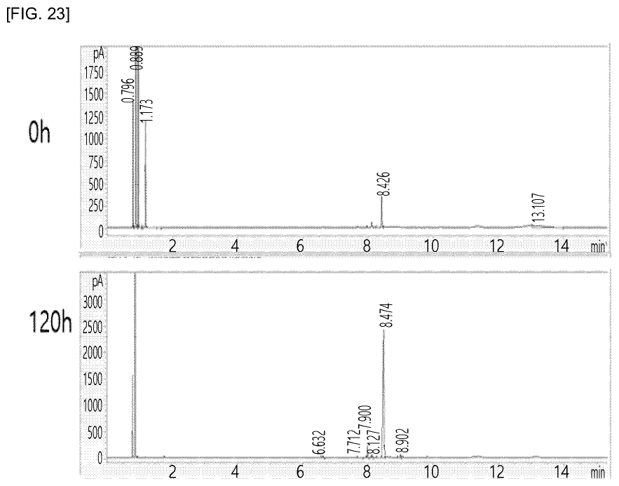

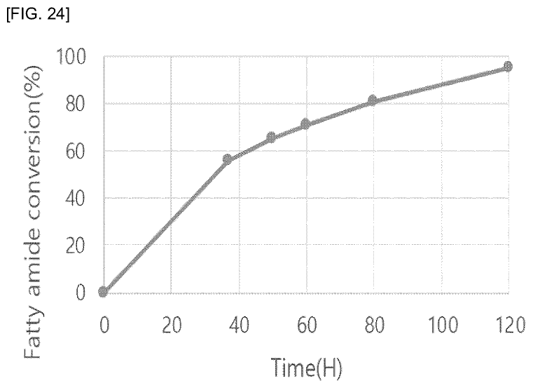

An amidation reaction was carried out and analyzed using a method similar to that of Example 1-1, except that evening primrose oil (EPO) was used instead of SFO, and the reaction was conducted for 96 hours. The fatty acid composition of EPO consists of 74% linoleic acid, 9.3% linolenic acid, 7% oleic acid, 6.4% palmitic acid, 2% stearic acid, etc. The GC peaks observed after the AP amidation reaction were identified as palmitic acid AP amide at RT 7.60 minutes, the three fatty acid amides-stearic acid AP amide, oleic acid AP amide, and linoleic acid AP amide-having the same carbon chain length at RT 8.11 minutes, and linolenic acid AP amide at RT 8.18 minutes (FIG. 23). The final AP amide conversion of EPO was 95% after 120 hours (FIG. 24). The characteristics and composition of the resulting fatty acid AP amides are presented in Table 12.

TABLE 12

| | | | Molecular | |
|---|---|---|---|---|
| Fatty acid AP | | Chemical | Weight | Content |
| amide | Structure | Formula | (Da) | (%) |
| Linoleamide-AP | 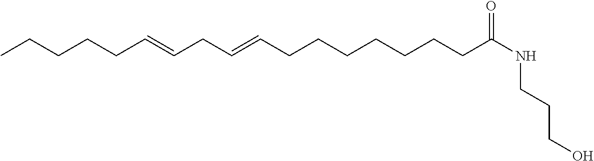 [SMILES: CCCCC/C=C/C/C=C/CCCCCCCC(=O)NCCCO] | C₂₁H₃₉NO₂ | 337.55 | ~74 |
| Linolenamide-AP | 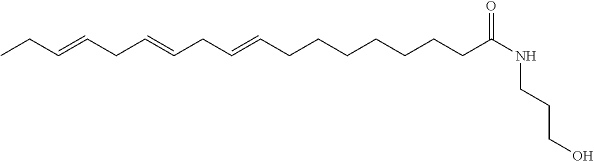 [SMILES: CC/C=C/C/C=C/C/C=C/CCCCCCCC(=O)NCCCO] | C₂₁H₃₇NO₂ | 335.53 | ~9.3 |
| Oleamide-AP | 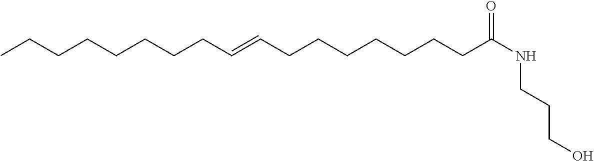 [SMILES: CCCCCCCC/C=C/CCCCCCCC(=O)NCCCO] | C₂₁H₄₁NO₂ | 339.56 | ~7.0 |
| Palmitamide-AP | 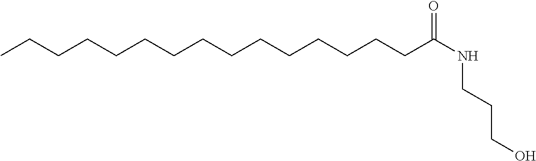 [SMILES: CCCCCCCCCCCCCCCC(=O)NCCCO] | C₁₉H₃₉NO₂ | 313.53 | ~6.4 |
| Stearamide-AP | 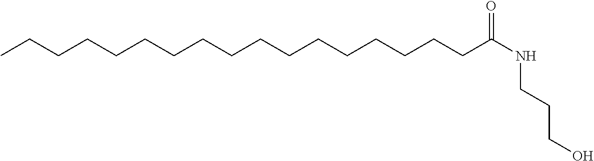 [SMILES: CCCCCCCCCCCCCCCCCC(=O)NCCCO] | C₂₁H₄₃NO₂ | 341.58 | ~2.0 |

### 1-13. Preparation of Endo-Cannabinoid Analogues (RSO-AP) Using Rapeseed Oil and 3-Amino-1-Propanol

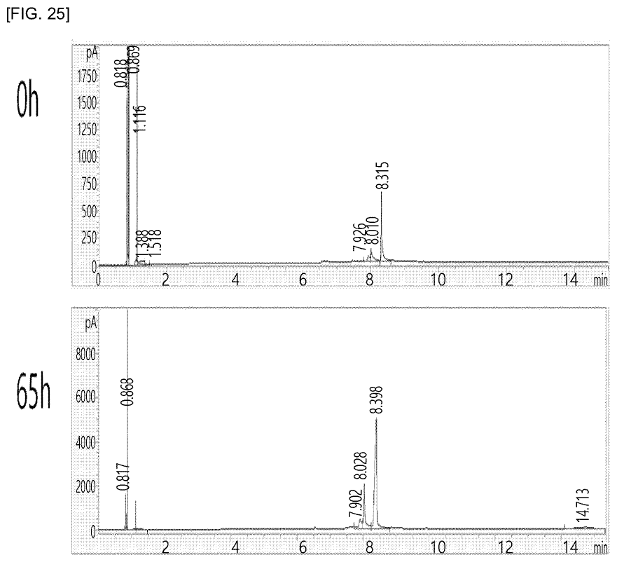

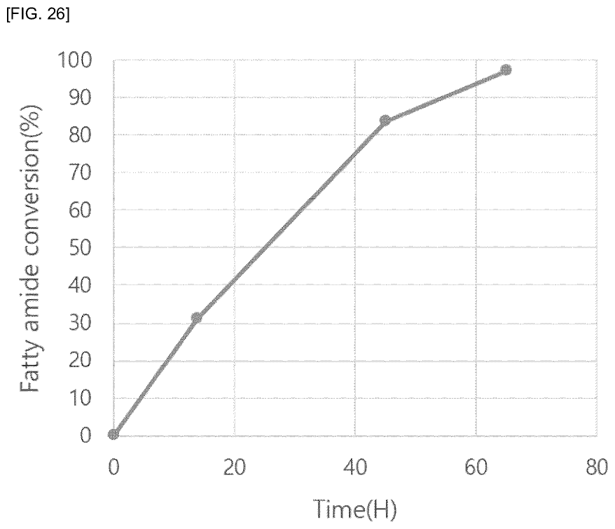

An amidation reaction was carried out and analyzed using a method similar to that of Example 1-1, except that rapeseed oil (RSO) was used instead of SFO, and the reaction was conducted for 96 hours. The fatty acid composition of RSO consists of 61.2% oleic acid, 18.8% linoleic acid, 10.8% linolenic acid, 4.2% palmitic acid, 1.7% stearic acid, etc. The GC peaks observed after the AP amidation reaction were identified as palmitic acid AP amide at RT 8.02 minutes, and the four fatty acid amides—stearic acid AP amide, oleic acid AP amide, linoleic acid AP amide, and linolenic acid AP amide—having the same carbon chain length at RT 8.39 minutes (FIG. 25). The final AP amide conversion of RSO was 97% after 65 hours (FIG. 26). The characteristics and composition of the resulting fatty acid AP amides are presented in Table 13.

TABLE 13

| | | | Molecular | |
|---|---|---|---|---|
| Fatty acid AP | | Chemical | Weight | Content |
| amide | Structure | Formula | (Da) | (%) |
| Oleamide-AP | 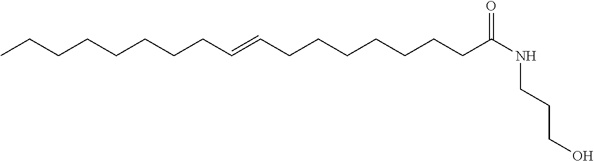 [SMILES: CCCCCCCC/C=C/CCCCCCCC(=O)NCCCO] | C₂₁H₄₁NO₂ | 339.56 | ~61.2 |
| Linoleamide-AP | 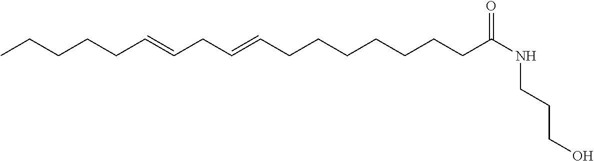 [SMILES: CCCCC/C=C/C/C=C/CCCCCCCC(=O)NCCCO] | C₂₁H₃₉NO₂ | 337.55 | ~18.8 |
| Linolenamide-AP | 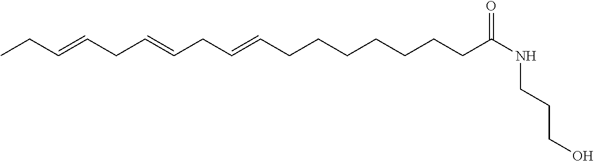 [SMILES: CC/C=C/C/C=C/C/C=C/CCCCCCCC(=O)NCCCO] | C₂₁H₃₇NO₂ | 335.53 | ~10.8 |
| Palmitamide-AP | 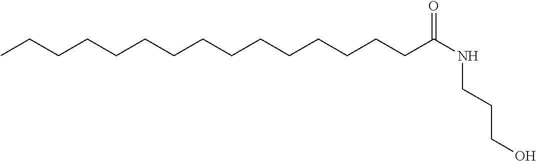 [SMILES: CCCCCCCCCCCCCCCC(=O)NCCCO] | C₁₉H₃₉NO2 | 313.53 | ~4.2 |
| Stearamide-AP | 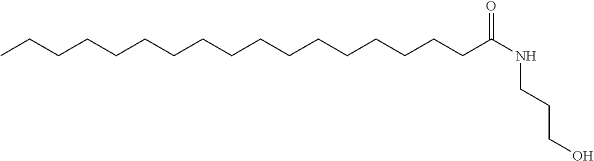 [SMILES: CCCCCCCCCCCCCCCCCC(=O)NCCCO] | C₂₁H₄₃NO₂ | 341.58 | ~1.7 |

### 1-14. Preparation of Endo-Cannabinoid Analogues (RSO-EA) Using Rapeseed Oil and Ethanolamine

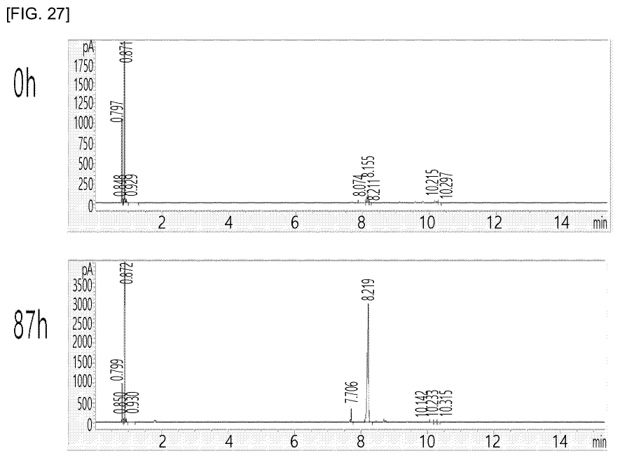

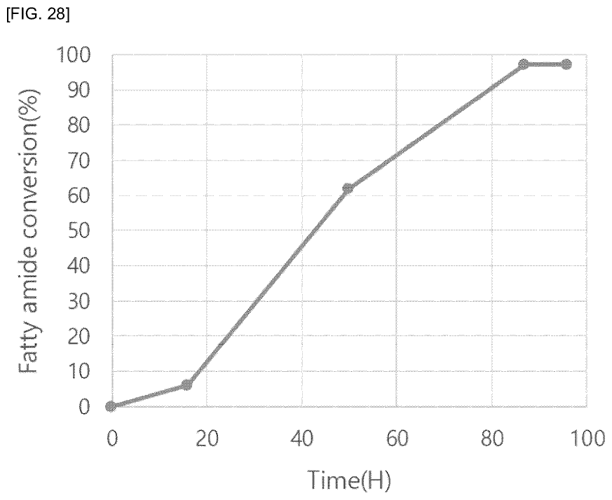

An amidation reaction was carried out and analyzed using a method similar to that of Example 1-13, except that EA was used instead of AP, and the reaction was conducted for 96 hours. The GC peaks observed after the EA amidation reaction were identified as palmitic acid EAamide at RT 7.7 minutes, the three fatty acid amides-stearic acid EA amide, oleic acid EA amide, and linoleic acid EA amide-having the same carbon chain length at the same peak at RT 8.21 minutes, and linolenic acid EA amide at RT 8.24 minutes (FIG. 27). The final EA amide conversion of RSO was 97% after 96 hours (FIG. 28). The characteristics and composition of the resulting fatty acid EA amides are presented in Table 14.

TABLE 14

| | | | Molecular | |
|---|---|---|---|---|
| Fatty acid EA | | Chemical | Weight | Content |
| amide | Structure | Formula | (Da) | (%) |
| Oleamide-EA | 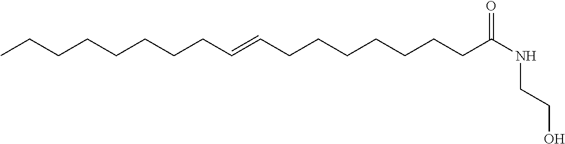 [SMILES: CCCCCCCC/C=C/CCCCCCCC(=O)NCCO] | C₂₀H₃₉NO₂ | 325.54 | ~61.2 |
| Linoleamide-EA | 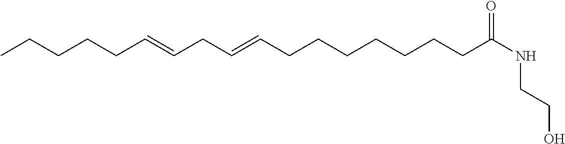 [SMILES: CCCCC/C=C/C/C=C/CCCCCCCC(=O)NCCO] | C₂₀H₃₇NO₂ | 323.52 | ~18.8 |
| Linolenamide-EA | 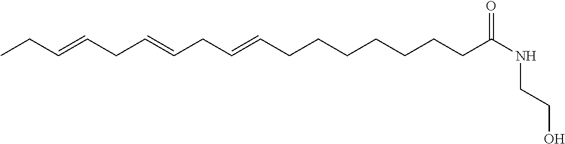 [SMILES: CC/C=C/C/C=C/C/C=C/CCCCCCCC(=O)NCCO] | C₂₀H₃₅NO₂ | 321.50 | ~10.8 |
| Palmitamide-EA | 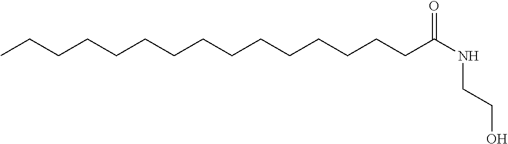 [SMILES: CCCCCCCCCCCCCCCC(=O)NCCO] | C₁₈H₃₇NO₂ | 299.50 | ~4.2 |
| Stearamide-EA | 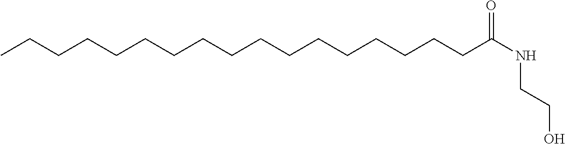 [SMILES: CCCCCCCCCCCCCCCCCC(=O)NCCO] | C₂₀H₄₁NO₂ | 327.55 | ~1.7 |

### 1-15. Preparation of Endo-Cannabinoid Analogues (MFO-AP) Using Meadowfoam Seed Oil and 3-Amino-1-Propanol

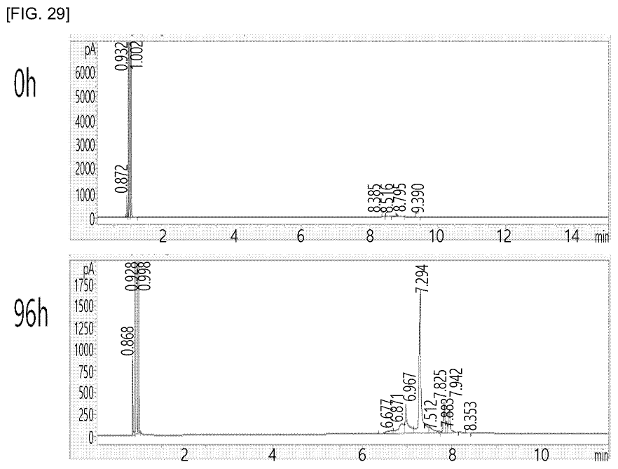

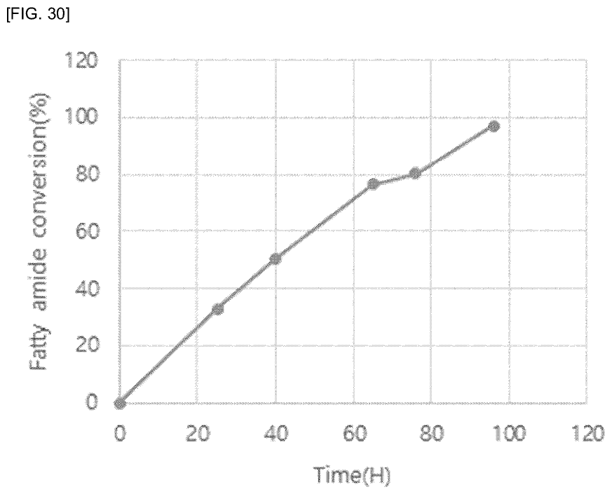

An amidation reaction was carried out and analyzed using a method similar to that of Example 1-1, except that meadowfoam seed oil (MFO) was used instead of SFO, and the reaction was conducted for 96 hours. The fatty acid composition of MFO consists of 60% eicosenoic acid, 18% erucic acid, 18% docosadienoic acid, etc. The GC peaks observed after the AP amidation reaction were identified as eicosenoic acid AP amide at RT 7.29 minutes, erucic acid AP amide at RT 7.82 minutes, and docosadienoic acid AP amide at RT 7.94 minutes, etc. (FIG. 29). The final AP amide conversion of MFO was 97% after 96 hours (FIG. 30). The characteristics and composition of the resulting fatty acid AP amides are presented in Table 15.

TABLE 15

| | | | Molecular | |
|---|---|---|---|---|
| | | Chemica | Weight | Content |
| Fatty acid AP amide | Structure | Formula | (Da) | (%) |
| Eicosenamide-AP | 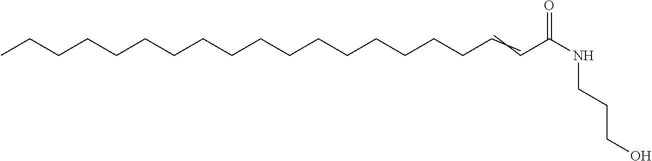 [SMILES: CCCCCCCCCCCCCCCCCC=CC(=O)NCCCO] | C₂₃H₄₅NO₂ | 367.62 | ~60.0 |
| Erucamide-AP | 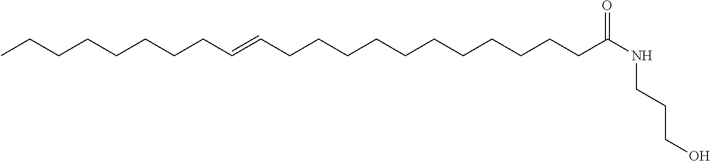 [SMILES: CCCCCCCC/C=C/CCCCCCCCCCCC(=O)NCCCO] | C₂₅H₄₉NO₂ | 395.67 | ~18.0 |
| Docosadienamide-AP | 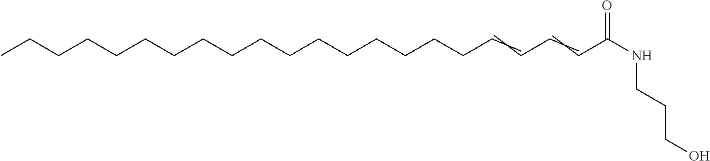 [SMILES: CCCCCCCCCCCCCCCCCC=CC=CC(=O)NCCCO] | C₂₅H₄₇NO₂ | 393.66 | ~18.0 |

### 1-16. Preparation of Endo-Cannabinoid Analogues (MFO-EA) Using Meadowfoam Seed Oil and Ethanolamine

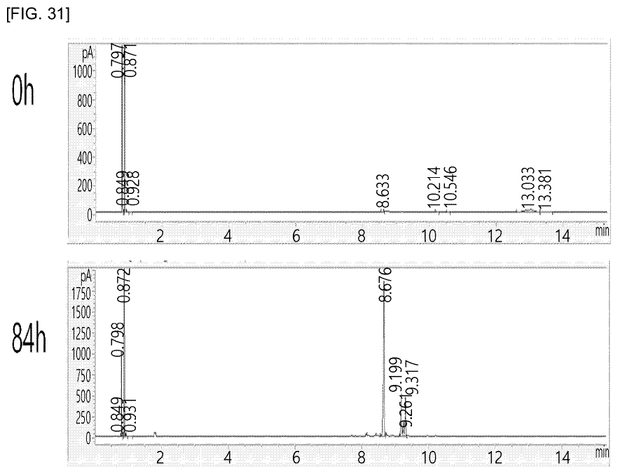

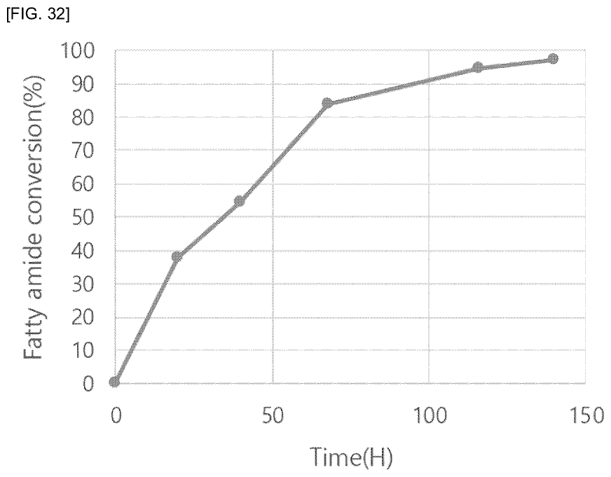

An amidation reaction was carried out and analyzed using a method similar to that of Example 1-15, except that EA was used instead of AP, and the reaction was conducted for 96 hours. The GC peaks observed after the EA amidation reaction were identified as eicosenoic acid EA amide at RT 8.67 minutes, erucic acid EA amide at RT 9.19 minutes, and docosadienoic acid EA amide at RT 9.32 minutes (FIG. 31). The final EA amide conversion rate of MSO was 96.5% after 84 hours (FIG. 32). The characteristics and composition of the resulting fatty acid EA amides are presented in Table 16.

TABLE 16

| | | | Molecular | |
|---|---|---|---|---|
| | | Chemical | Weight | Content |
| Fatty acid EA amide | Structure | Formula | (Da) | (%) |
| Eicosenamide-EA | 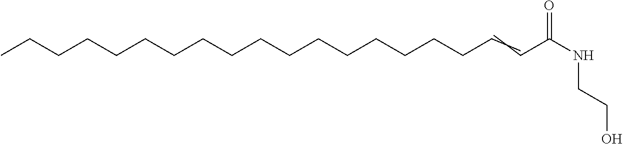 [SMILES: CCCCCCCCCCCCCCCCCC=CC(=O)NCCO] | C₂₂H₄₃NO₂ | 353.59 | ~60.0 |
| Erucamide-EA | 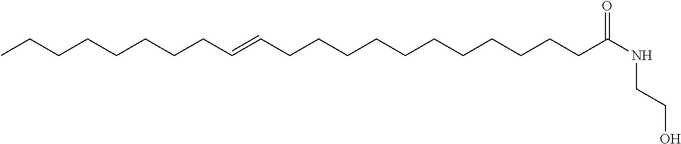 [SMILES: CCCCCCCC/C=C/CCCCCCCCCCCC(=O)NCCO] | C₂₄H₄₇NO₂ | 381.64 | ~18.0 |
| Docosadienamide- EA | 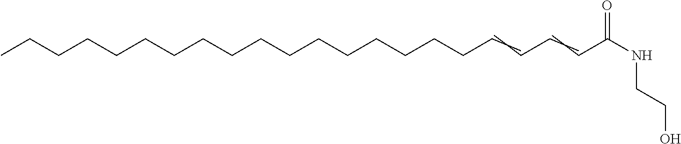 [SMILES: CCCCCCCCCCCCCCCCCC=CC=CC(=O)NCCO] | C₂₄H₄₅NO₂ | 379.63 | ~18.0 |

### 1-17. Preparation of Endo-Cannabinoid Analogues (ABSO-AP) Using abyssinica Seed Oil and 3-Amino-1-Propanol

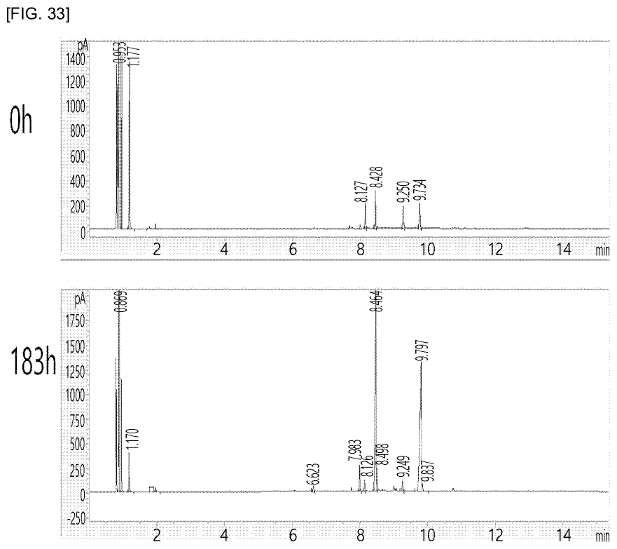

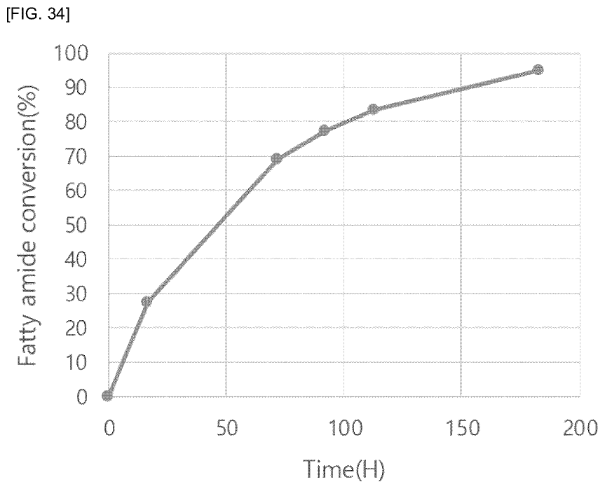

An amidation reaction was carried out and analyzed using a method similar to that of Example 1-1, except that abyssinica seed oil (ABSO) was used instead of SFO, and the reaction was conducted for 96 hours. The fatty acid composition of ABSO consists of 63.8% erucic acid, 15.1% oleic acid, 13.2% linoleic acid, 2.4% eicosenoic acid, 2.1% behenic acid, etc. The GC peaks observed after the AP amidation reaction were identified as oleic acid AP amide and linoleic acid AP amide at RT 8.46 minutes, and erucic acid AP amide, eicosenoic acid AP amide, and behenic acid AP amide at RT 9.79 minutes (FIG. 33). The final AP amide conversion of ABSO was 95.5% after 183 hours (FIG. 34). The characteristics and composition of the resulting fatty acid AP amides are presented in Table 17.

TABLE 17

| | | Chemical | Molecular | Content |
|---|---|---|---|---|
| Fatty acid AP amide | Structure | Formula | Weight (Da) | (%) |
| Eicosenamide-AP | 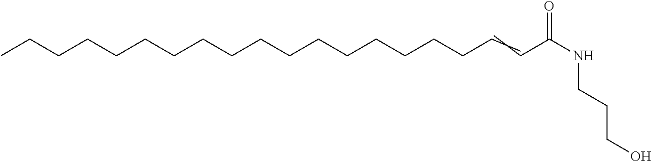 [SMILES: CCCCCCCCCCCCCCCCCC=CC(=O)NCCCO] | C₂₃H₄₅NO₂ | 367.62 | ~63.8 |
| Oleamide-AP | 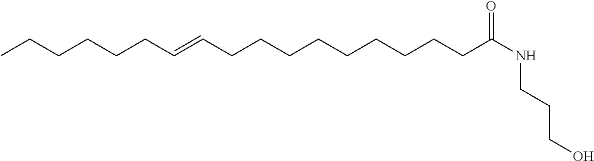 [SMILES: CCCCCC/C=C/CCCCCCCCCC(=O)NCCCO] | C₂₁H₄₁NO₂ | 339.56 | ~15.1 |
| Linoleamide-AP | 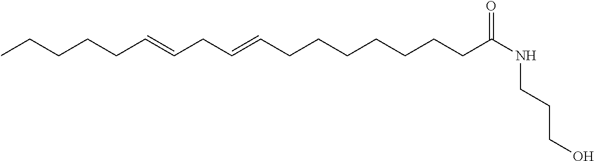 [SMILES: CCCCC/C=C/C/C=C/CCCCCCCC(=O)NCCCO] | C₂₁H₃₉NO₂ | 337.55 | ~13.2 |
| Erucamide-AP | 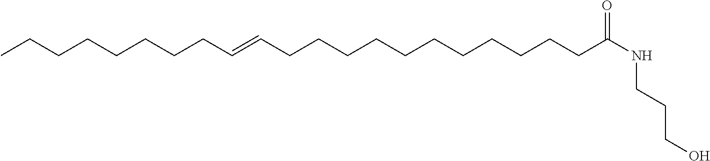 [SMILES: CCCCCCCC/C=C/CCCCCCCCCCCC(=O)NCCCO] | C₂₅H₄₉NO₂ | 395.67 | ~2.4 |
| Behenamide-AP | 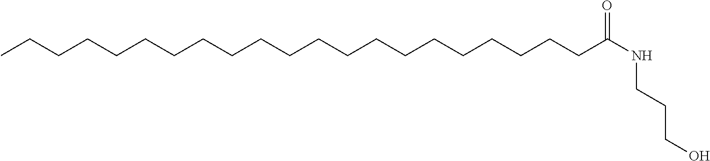 [SMILES: CCCCCCCCCCCCCCCCCCCCCC(=O)NCCCO] | C₂₅H₅₁NO₂ | 397.69 | ~2.1 |

### 1-18. Preparation of Endo-Cannabinoid Analogues (HSO-AP) Using Hempseed Oil and 3-Amino-1-Propanol

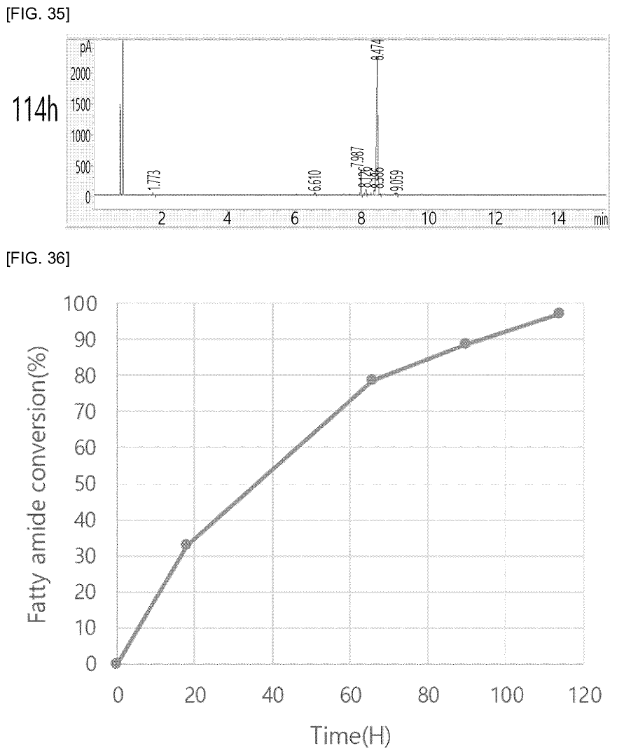

An amidation reaction was carried out and analyzed using a method similar to that of Example 1-1, except that hempseed oil (HSO) was used instead of SFO, and the reaction was conducted for 96 hours. The fatty acid composition of HSO consists of 56% linoleic acid, 24% linolenic acid, 9% oleic acid, 6% palmitic acid, 2.5% stearic acid, 2.5% eicosenoic acid, etc. The GC peaks observed following the AP amidation reaction were identified as linoleic acid AP amide, linolenic acid AP amide, oleic acid AP amide, and stearic acid AP amide at RT 8.47 minutes, palmitic acid AP amide at RT 7.98 minutes, eicosenoic acid AP amide at RT 9.05 minutes, etc. (FIG. 35). The final AP amide conversion of HSO was 97.2% after 113 hours (FIG. 36). The characteristics and composition of the resulting fatty acid AP amides are presented in Table 18.

TABLE 18

| | | | Molecular | |
|---|---|---|---|---|
| Fatty acid AP | | Chemical | Weight | Content |
| amide | Structure | Formula | (Da) | (%) |
| Linoleamide-AP | 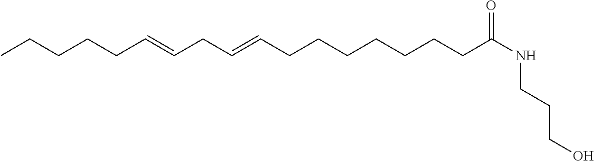 [SMILES: CCCCC/C=C/C/C=C/CCCCCCCC(=O)NCCCO] | C₂₁H₃₉NO₂ | 337.55 | ~56.0 |
| Linolenamide-AP | 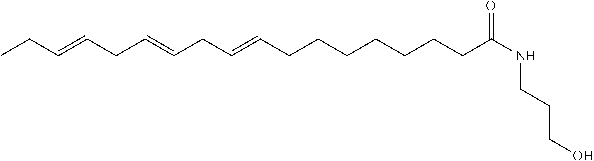 [SMILES: CC/C=C/C/C=C/C/C=C/CCCCCCCC(=O)NCCCO] | C₂₁H₃₇NO₂ | 335.53 | ~24.0 |
| Oleamide-AP | 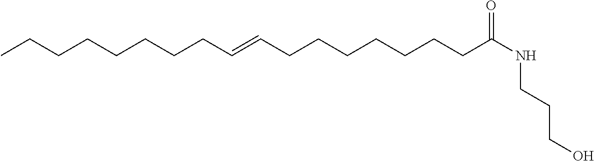 [SMILES: CCCCCCCC/C=C/CCCCCCCC(=O)NCCCO] | C₂₁H₄₁NO₂ | 339.56 | ~9.0 |
| Palmitamide-AP | 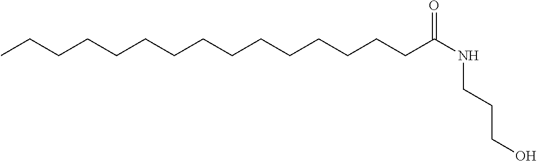 [SMILES: CCCCCCCCCCCCCCCC(=O)NCCCO] | C₁₉H₃₉NO₂ | 313.53 | ~6.0 |
| Stearamide-AP | 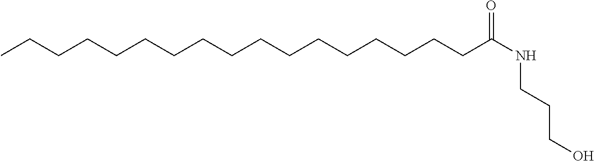 [SMILES: CCCCCCCCCCCCCCCCCC(=O)NCCCO] | C₂₁H₄₃NO₂ | 341.58 | ~2.5 |
| Eicosenamide-AP | 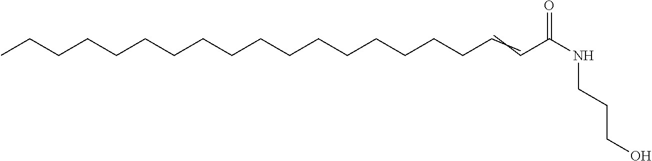 [SMILES: CCCCCCCCCCCCCCCCCC=CC(=O)NCCCO] | C₂₃H₄₅NO₂ | 367.62 | ~2.5 |

### 1-19. Preparation of Endo-Cannabinoid Analogues (AVO-AP) Using Avocado Oil and 3-Amino-1-Propanol

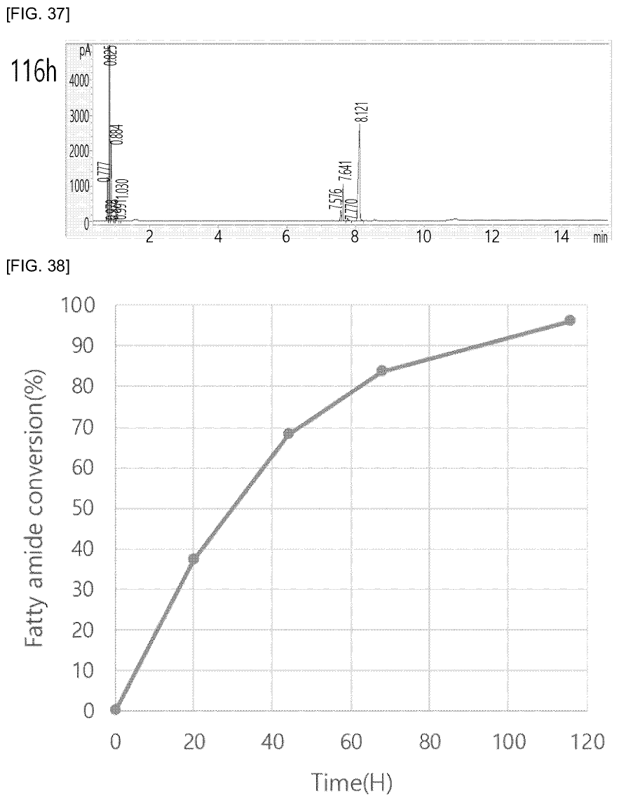

An amidation reaction was carried out and analyzed using a method similar to that of Example 1-1, except that avocado oil (AVO) was used instead of SFO, and the reaction was conducted for 96 hours. The fatty acid composition of AVO consists of 51.0% oleic acid, 28.2% palmitic acid, 13.0% linoleic acid, 0.7% stearic acid, 0.6% linolenic acid, etc. The GC peaks observed following the AP amidation reaction were identified as oleic acid AP amide, linoleic acid AP amide, stearic acid AP amide, and linolenic acid AP amide at RT 8.12 minutes, palmitic acid AP amide at RT 7.64 minutes, etc. (FIG. 37). The final AP amide conversion of AVO was 96% after 116 hours (FIG. 38). The characteristics and composition of the resulting fatty acid AP amides are presented in Table 19.

TABLE 19

| | | | Molecular | |
|---|---|---|---|---|
| Fatty acid AP | | Chemical | Weight | Content |
| amide | Structure | Formula | (Da) | (%) |
| Oleamide-AP | 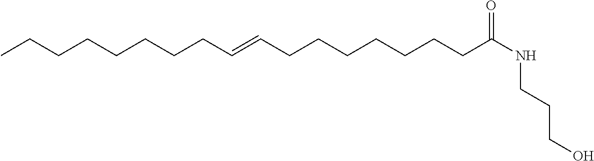 [SMILES: CCCCCCCC/C=C/CCCCCCCC(=O)NCCCO] | C₂₁H₄₁NO₂ | 339.56 | ~51.0 |
| Palmitamide-AP | 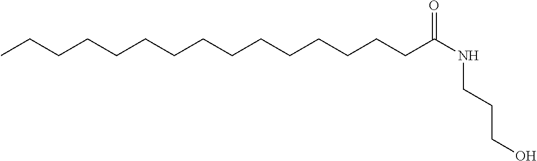 [SMILES: CCCCCCCCCCCCCCCC(=O)NCCCO] | C₁₉H₃₉NO₂ | 313.53 | ~28.2 |
| Linoleamide-AP | 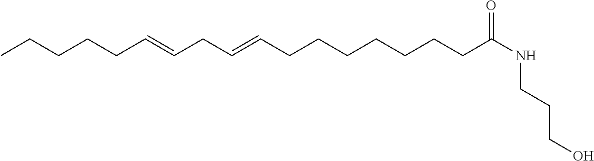 [SMILES: CCCCC/C=C/C/C=C/CCCCCCCC(=O)NCCCO] | C₂₁H₃₉NO₂ | 337.55 | ~13.0 |
| Stearamide-AP | 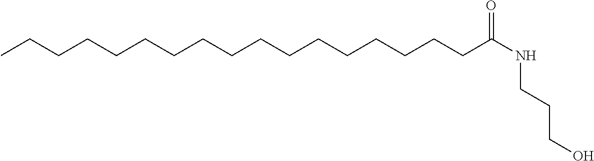 [SMILES: CCCCCCCCCCCCCCCCCC(=O)NCCCO] | C₂₁H₄₃NO₂ | 341.58 | ~0.7 |
| Linolenamide-AP | 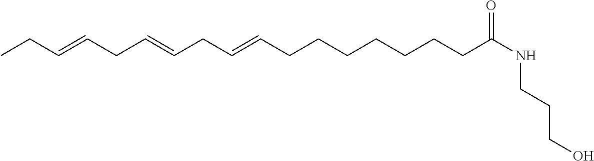 [SMILES: CC/C=C/C/C=C/C/C=C/CCCCCCCC(=O)NCCCO] | C₂₁H₃₇NO₂ | 335.53 | ~0.6 |

### 1-20. Preparation of Endo-Cannabinoid Analogues (RBO-AP) Using Rice Bran Oil and 3-Amino-1-Propanol

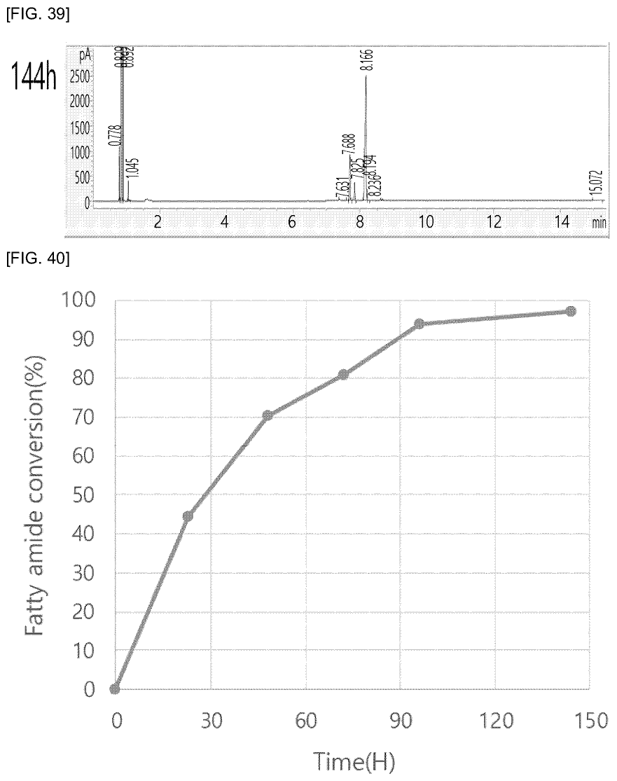

An amidation reaction was carried out and analyzed using a method similar to that of Example 1-1, except that rice bran oil (RBO) was used instead of SFO, and the reaction was conducted for 96 hours. The fatty acid composition of RBO consists of 49.1% oleic acid, 33.2% linoleic acid, 16.0% palmitic acid, 1.7% linolenic acid, etc. The GC peaks observed following the AP amidation reaction were identified as oleic acid AP amide, linoleic acid AP amide, and linolenic acid AP amide at RT 8.16 minutes, palmitic acid AP amide at RT 7.68 minutes, etc. (FIG. 39). The final AP amide conversion of RBO was 97.2% after 114 hours (FIG. 40). The characteristics and composition of the resulting fatty acid AP amides are presented in Table 20.

TABLE 20

| | | | Molecular | |
|---|---|---|---|---|
| Fatty acid AP | | Chemical | Weight | Content |
| amide | Structure | Formula | (Da) | (%) |
| Oleamide-AP | 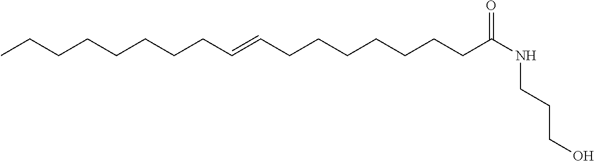 [SMILES: CCCCCCCC/C=C/CCCCCCCC(=O)NCCCO] | C₂₁H₄₁NO₂ | 339.56 | ~49.1 |
| Linoleamide-AP | 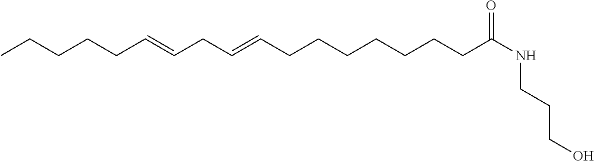 [SMILES: CCCCC/C=C/C/C=C/CCCCCCCC(=O)NCCCO] | C₂₁H₃₉NO₂ | 337.55 | ~33.2 |
| Palmitamide-AP | 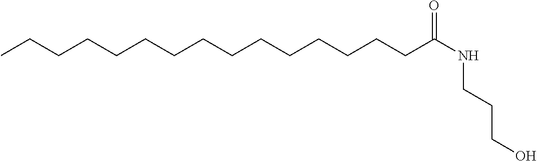 [SMILES: CCCCCCCCCCCCCCCC(=O)NCCCO] | C₁₉H₃₉NO₂ | 313.53 | ~16.0 |
| Linolenamide-AP | 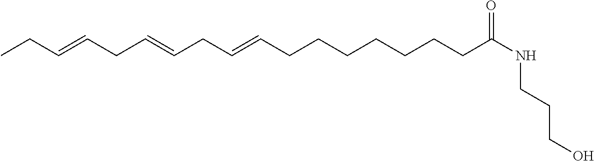 [SMILES: CC/C=C/C/C=C/C/C=C/CCCCCCCC(=O)NCCCO] | C₂₁H₃₇NO₂ | 335.53 | ~1.7 |

### 1-21. Preparation of Endo-Cannabinoid Analogues (SHB-AP) Using Shea Butter and 3-Amino-1-Propanol

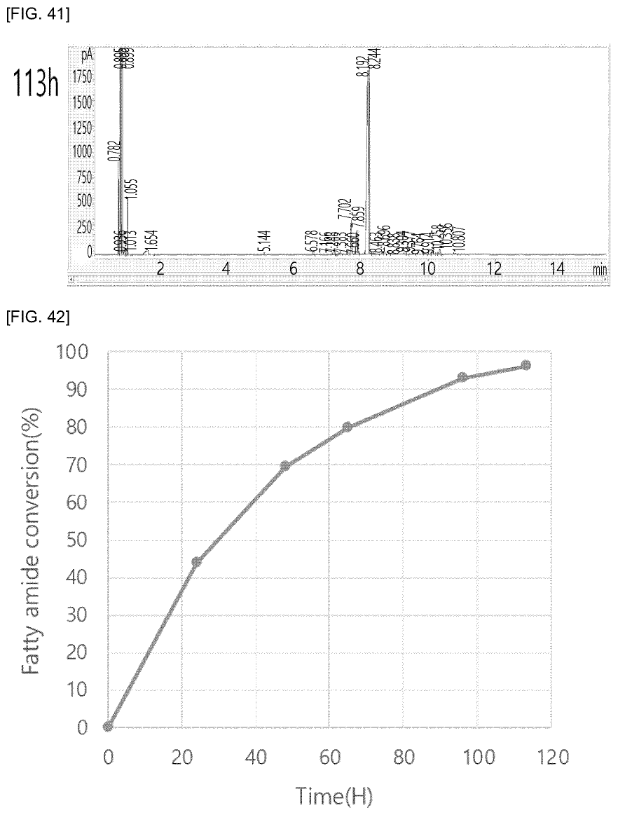

An amidation reaction was carried out and analyzed using a method similar to that of Example 1-1, except that shea butter (SHB) was used instead of SFO, and the reaction was conducted for 96 hours. The fatty acid composition of SHB consists of 52.4% stearic acid, 36.3% oleic acid, 5.4% linoleic acid, 3.0% palmitic acid, etc. The GC peaks observed following the AP amidation reaction were identified as stearic acid AP amide, oleic acid AP amide, and linoleic acid AP amide at RT 8.24 minutes, palmitic acid AP amide at RT 7.7 minutes, etc. (FIG. 41). The final AP amide conversion of SHB was 97.2% after 114 hours (FIG. 42). The characteristics and composition of the resulting fatty acid AP amides are presented in Table 21.

TABLE 21

| | | | Molecular | |
|---|---|---|---|---|
| Fatty acid | | Chemical | Weight | Content |
| AP amide | Structure | Formula | (Da) | (%) |
| Stearamide- AP | 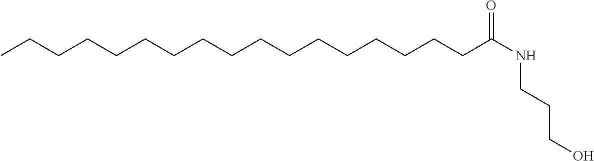 [SMILES: CCCCCCCCCCCCCCCCCC(=O)NCCCO] | C₂₁H₄₃NO₂ | 341.58 | ~52.4 |
| Oleamide- AP | 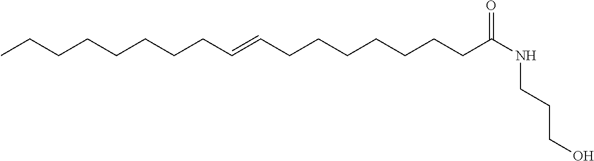 [SMILES: CCCCCCCC/C=C/CCCCCCCC(=O)NCCCO] | C₂₁H₄₁NO₂ | 339.56 | ~36.3 |
| Linoleamide- AP | 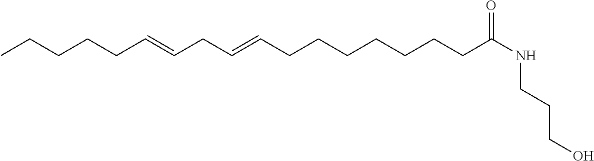 [SMILES: CCCCC/C=C/C/C=C/CCCCCCCC(=O)NCCCO] | C₂₁H₃₉NO₂ | 337.55 | ~5.4 |
| Palmitamide- AP | 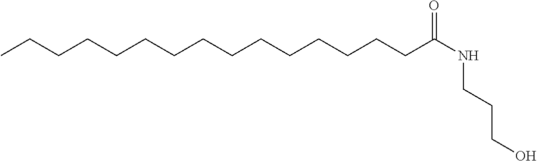 [SMILES: CCCCCCCCCCCCCCCC(=O)NCCCO] | C₁₉H₃₉NO₂ | 313.53 | ~3.0 |

### 1-22. Preparation of Endo-Cannabinoid Analogues (ATO-AP) Using Aurantiochytrium Oil and 3-Amino-1-Propanol

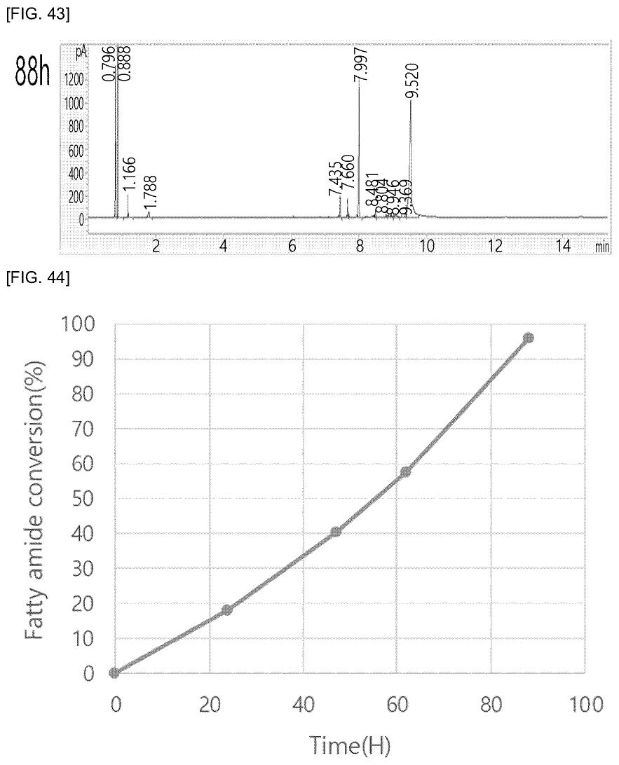

An amidation reaction was carried out and analyzed using a method similar to that of Example 1-1, except that Aurantiochytrium oil (ATO) was used instead of SFO, and the reaction was conducted for 96 hours. The fatty acid composition of ATO consists of 54.0% docosahexaenoic acid, 37.0% palmitic acid, 5.0% myristic acid, 2.0% oleic acid, 2.0% eicosapentaenoic acid, etc. The GC peaks observed following the AP amidation reaction were identified as docosahexaenoic acid AP amide at RT 9.52 minutes, palmitic acid AP amide at RT 7.99 minutes, etc. (FIG. 43). The final AP amide conversion of ATO was 96% after 88 hours (FIG. 44). The characteristics and composition of the resulting fatty acid AP amides are presented in Table 22.

TABLE 22

| | | | Molecular | |
|---|---|---|---|---|
| | | Chemical | Weight | Content |
| Fatty acid AP amide | Structure | Formula | (Da) | (%) |
| Docosahexaenamide- AP | 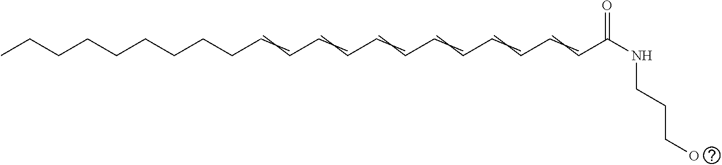 [SMILES: CCCCCCCCCC=CC=CC=CC=CC=CC=CC(=O)NCCCO] | C₂₅H₃₉NO₂ | 385.59 | ~54.0 |
| Palmitamide-AP | 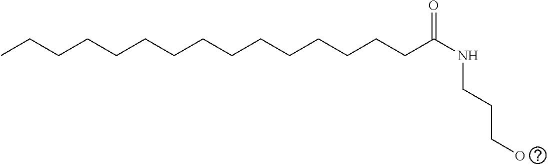 [SMILES: CCCCCCCCCCCCCCCC(=O)NCCCO] | C₁₉H₃₉NO₂ | 313.53 | ~37.0 |
| Myristamide-AP | 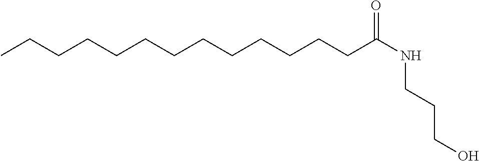 [SMILES: CCCCCCCCCCCCCC(=O)NCCCO] | C₁₇H₃₅NO₂ | 285.47 | ~5 |
| Oleamide-AP | 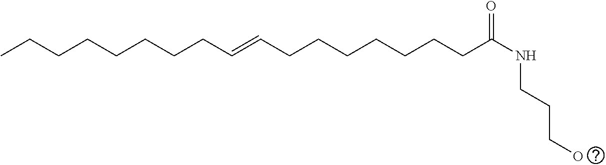 [SMILES: CCCCCCCC/C=C/CCCCCCCC(=O)NCCCO] | C₂₁H₄₁NO₂ | 339.56 | ~2.0 |
| Eicosapentaenamide- AP | 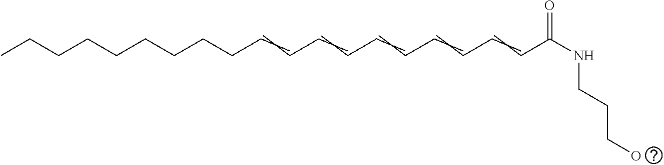 [SMILES: CCCCCCCCCC=CC=CC=CC=CC=CC(=O)NCCCO] | C₂₃H₃₇NO₂ | 359.55 | ~2.0 |

### 1-23. Preparation of Endo-Cannabinoid Analogues (CAM-MIPA) Using Camelia Oil and Monoisopropanolamine

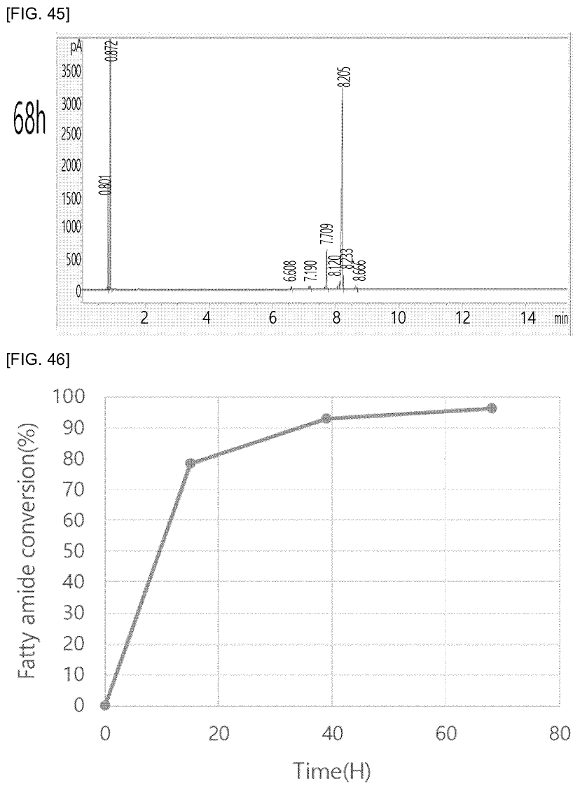

An amidation reaction was carried out and analyzed using a method similar to that of Example 1-3, except that camelia oil (CAM) was used instead of SFO, and the reaction was conducted for 48 hours. The fatty acid composition of CAM consists of 78.2% oleic acid, 9.6% palmitic acid, 9.1% linoleic acid, 2.0% stearic acid, etc. The GC peaks observed following the MIPA amidation reaction were identified as oleic acid MIPA amide, linoleic acid MIPA amide, and stearic acid MIPA amide at RT 8.2 minutes, palmitic acid MIPA amide at RT 7.7 minutes, etc. (FIG. 45). The final MIPA amide conversion of CAM was 96% after 68 hours (FIG. 46). The characteristics and composition of the resulting fatty acid MIPA amides are presented in Table 23.

TABLE 23

| Fatty acid | | Chemical | Molecular | Content |
|---|---|---|---|---|
| MIPA amide | Structure | Formula | Weight (Da) | (%) |
| Oleamide- MIPA | 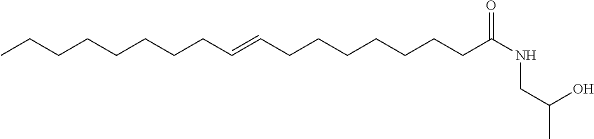 [SMILES: CCCCCCCC/C=C/CCCCCCCC(=O)NCC(C)O] | C₂₁H₄₁NO₂ | 339.56 | ~78.2 |
| Palmitamide- MIPA | 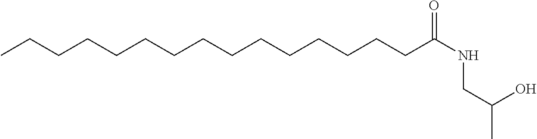 [SMILES: CCCCCCCCCCCCCCCC(=O)NCC(C)O] | C₁₉H₃₉NO₂ | 313.53 | ~9.6 |
| Linoleamide- MIPA | 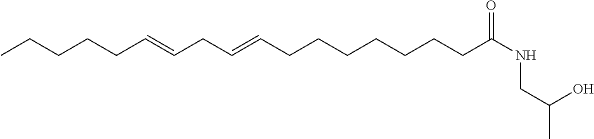 [SMILES: CCCCC/C=C/C/C=C/CCCCCCCC(=O)NCC(C)O] | C₂₁H₃₉NO₂ | 337.55 | ~9.1 |
| Stearamide- MIPA | 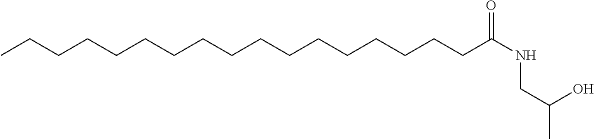 [SMILES: CCCCCCCCCCCCCCCCCC(=O)NCC(C)O] | C₂₁H₄₃NO₂ | 341.58 | ~2.0 |

### 1-24. Preparation of Endo-Cannabinoid Analogues (SFF-MIPA) Using Safflower Oil and Monoisopropanolamine

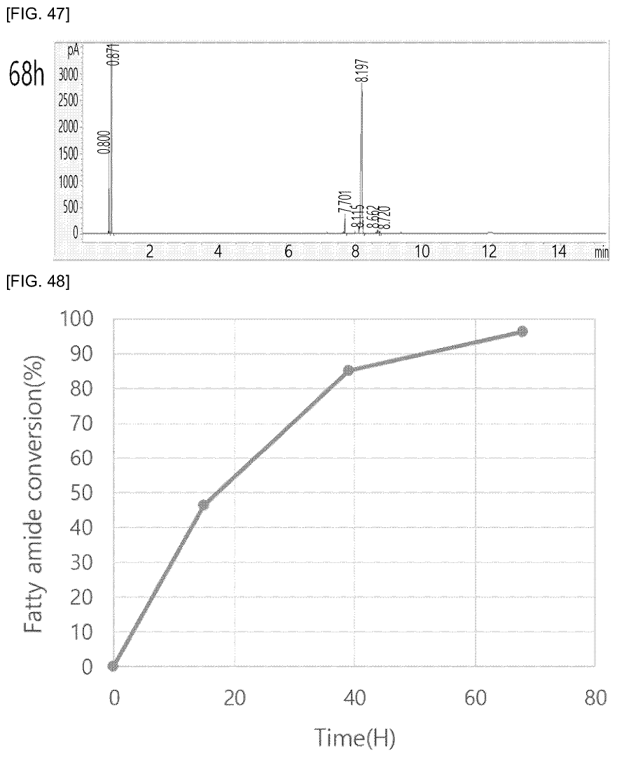

An amidation reaction was carried out and analyzed using a method similar to that of Example 1-3, except that safflower oil (SFF) was used instead of SFO, and the reaction was conducted for 48 hours. The fatty acid composition of SFF consists of 79.2% linoleic acid, 10.8% oleic acid, 6.2% palmitic acid, 2.0% stearic acid, etc. The GC peaks observed following the MIPA amidation reaction were identified as linoleic acid MIPA amide, oleic acid MIPA amide, and stearic acid MIPA amide at RT 8.19 minutes, and palmitic acid MIPA amide at RT 7.7 minutes (FIG. 47). The final MIPA amide conversion of SFF was 96.5% after 68 hours (FIG. 48). The characteristics and composition of the resulting fatty acid MIPA amides are presented in Table 24.

TABLE 24

| Fatty acid | | | Molecular | |
|---|---|---|---|---|
| MIPA | | Chemical | Weight | Content |
| amide | Structure | Formula | (Da) | (%) |
| Linoleamide- MIPA | 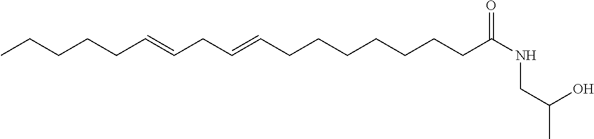 [SMILES: CCCCC/C=C/C/C=C/CCCCCCCC(=O)NCC(C)O] | C₂₁H₃₉NO₂ | 337.55 | ~79.2 |
| Oleamide- MIPA | 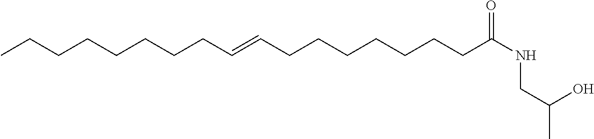 [SMILES: CCCCCCCC/C=C/CCCCCCCC(=O)NCC(C)O] | C₂₁H₄₁NO₂ | 339.56 | ~10.8 |
| Palmitamide- MIPA | 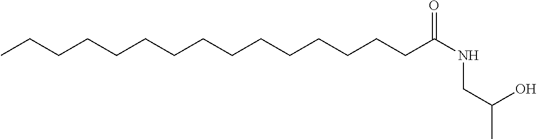 [SMILES: CCCCCCCCCCCCCCCC(=O)NCC(C)O] | C₁₉H₃₉NO₂ | 313.53 | ~6.2 |
| Stearamide- MIPA | 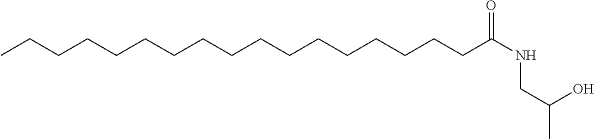 [SMILES: CCCCCCCCCCCCCCCCCC(=O)NCC(C)O] | C₂₁H₄₃NO₂ | 341.58 | ~2.0 |

### 1-25. Preparation of Endo-Cannabinoid Analogues (SBO-AMP) Using Soybean Oil and 2-Amino-2-Methyl-1-Propanol

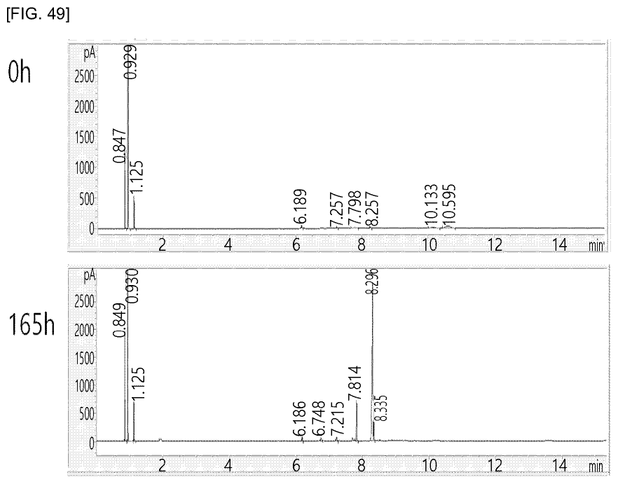

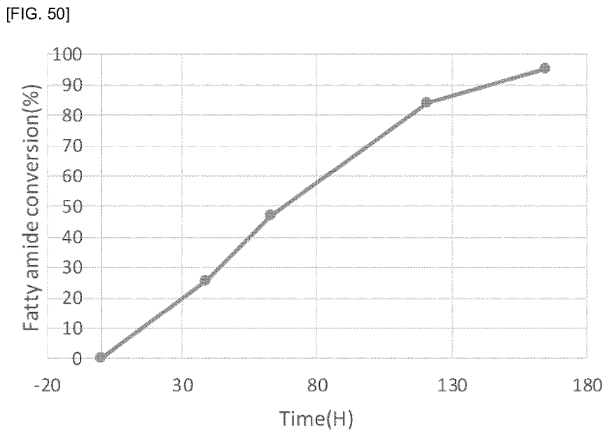

An amidation reaction was carried out and analyzed using a method similar to that of Example 1-4, except that SBO was used instead of SFO, and the reaction was conducted for 48 hours. The GC peaks observed following the AMP amidation reaction were identified as palmitic acid AMP amide at RT 7.81 minutes, the three fatty acid amides-stearic acid AMP amide, oleic acid AMP amide, and linoleic acid AMP amide-having the same carbon chain length at RT 8.29 minutes, and linolenic acid AMP amide at RT 8.33 minutes (FIG. 49). The final AMP amide conversion of SBO was 95% after 165 hours (FIG. 50). The characteristics and composition of the resulting fatty acid AMP amides are presented in Table 25.

TABLE 25

| | | | Molecular | |
|---|---|---|---|---|
| Fatty acid AMP | | Chemical | Weight | Content |
| amide | Structure | Formula | (Da) | (%) |
| Linoleamide-AMP | 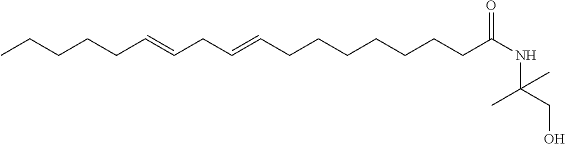 [SMILES: CCCCC/C=C/C/C=C/CCCCCCCC(=O)NC(C)(C)CO] | C₂₂H₄₁NO₂ | 351.57 | ~54 |
| Oleamide-AMP | 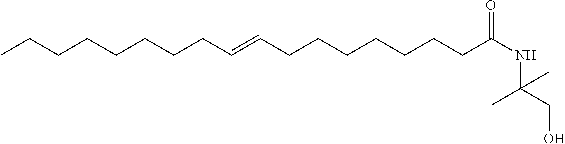 [SMILES: CCCCCCCC/C=C/CCCCCCCC(=O)NC(C)(C)CO] | C₂₂H₄₃NO₂ | 353.59 | ~24 |
| Palmitamide-AMP | 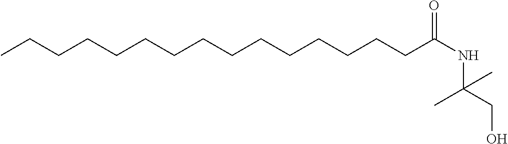 [SMILES: CCCCCCCCCCCCCCCC(=O)NC(C)(C)CO] | C₂₀H₄₁NO₂ | 327.55 | ~11 |
| Linolenamide-AMP | 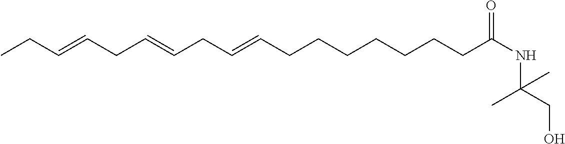 [SMILES: CC/C=C/C/C=C/C/C=C/CCCCCCCC(=O)NC(C)(C)CO] | C₂₂H₃₉NO₂ | 349.56 | ~7 |
| Stearamide-AMP | 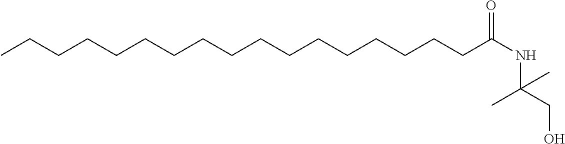 [SMILES: CCCCCCCCCCCCCCCCCC(=O)NC(C)(C)CO] | C₂₂H₄₅NO₂ | 355.35 | ~4 |

### Example 2. Preparation of Endo-Cannabinoid Analogues (SBO-EA) Using Fatty Acids

### 2-1. Preparation of Endo-Cannabinoid Analogues (Oleamide-EA) Using Oleic Acid and Ethanolamine

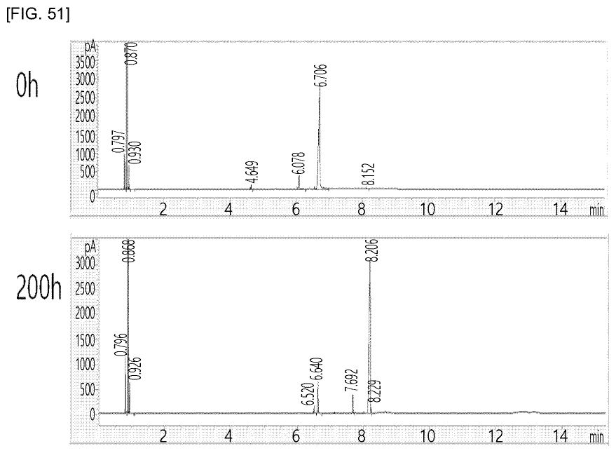

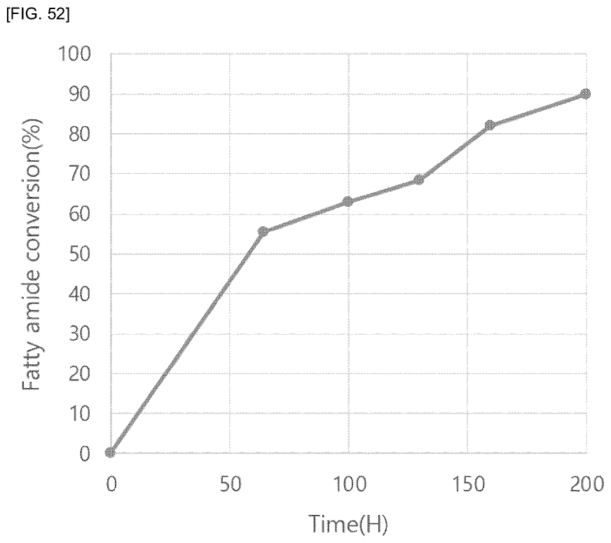

An amidation reaction was carried out and analyzed using a method similar to that of Example 1-2, except that oleic acid, a single fatty acid, was used instead of SFO, and the reaction was conducted with the same concentration of ethanolamine for 48 hours. The GC peak produced after the EA amidation reaction was identified as oleic acid EA amide at RT 8.2 minutes (FIG. 51). The final EA amide conversion of oleic acid was 90% after 200 hours (FIG. 52). The characteristics and composition of the resulting oleic acid EA amide are presented in Table 26.

TABLE 26

| Fatty acid EA | | Chemical | Molecular | Content |
|---|---|---|---|---|
| amide | Structure | Formula | Weight (Da) | (%) |
| Oleamide-EA | 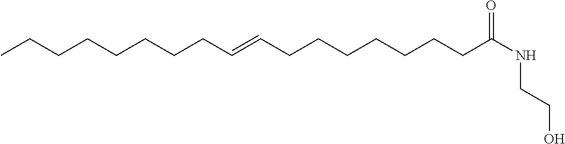 [SMILES: CCCCCCCC/C=C/CCCCCCCC(=O)NCCO] | C₂₀H₃₉NO₂ | 325.54 | ~90 |

### Example 3: Functional Evaluation of Endo-Cannabinoid Analogues

### 3-1. Evaluation of Binding Efficiency to Cannabinoid Receptors

In order to evaluate the functionality of the endo-cannabinoid analogues produced according to an embodiment of the present invention, the agonist effect on CB1 and CB2 was evaluated. The endo-cannabinoid analogues SFO-EA and SFO-AP, synthesized using sunflower oil and aminoalcohols, were used as samples. The efficacy was evaluated by measuring the amount of cyclic adenosine monophosphate (cAMP) generated from adenosine triphosphate (ATP) through the activation of adenylyl cyclase, which is triggered by G-protein alpha (G-protein a) as part of the stepwise signal transduction pathway initiated upon the binding of the test sample to the receptor. The efficacy was evaluated by measuring the amount of cyclic adenosine monophosphate (cAMP) generated from adenosine triphosphate (ATP) through G-protein alpha (G-protein a), which is activated by the binding of the sample to the receptor as part of the stepwise signal transduction pathway, and adenylyl cyclase, which is activated by G-protein a.

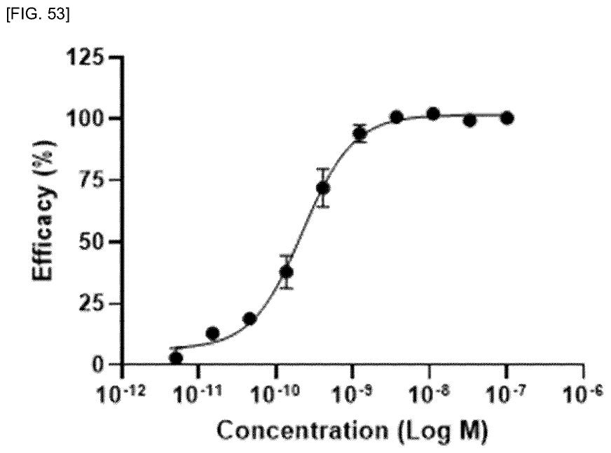

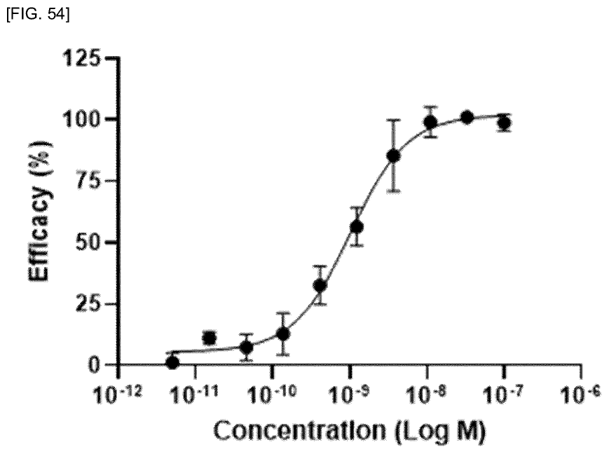

As shown in Table 27 below, the analysis results confirmed that the effective concentration 50 (EC50) of the positive control CP55940 for CB1 and CB2 was 0.222 nM (FIG. 53) and 1.019 nM (FIG. 54), respectively. This indicates that CP55940 has a higher binding efficiency for CB1 than for CB2.

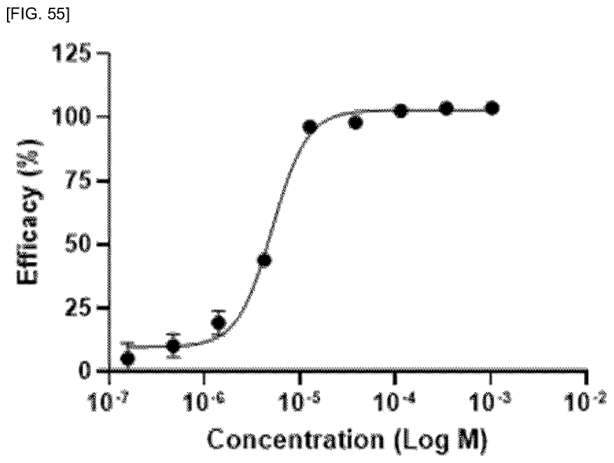

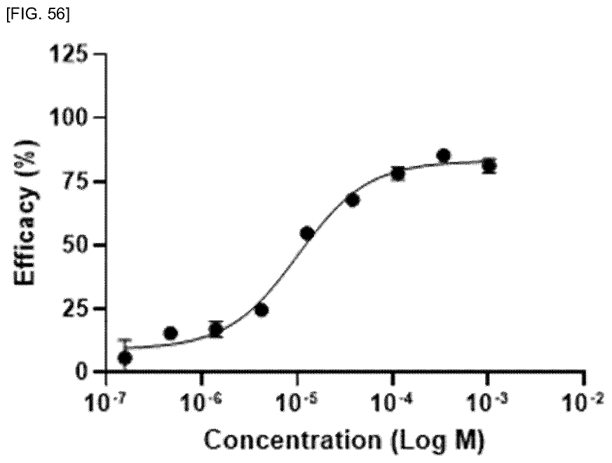

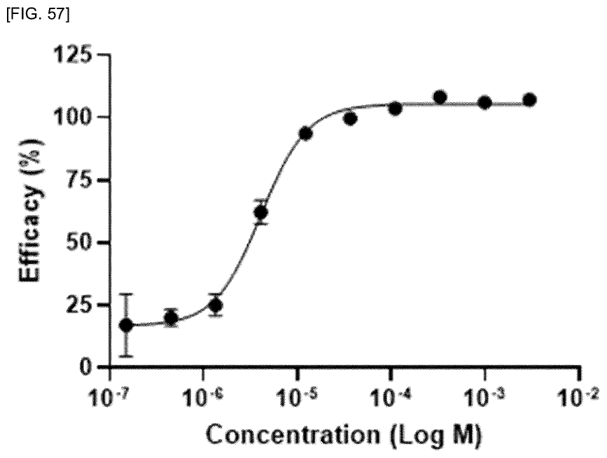

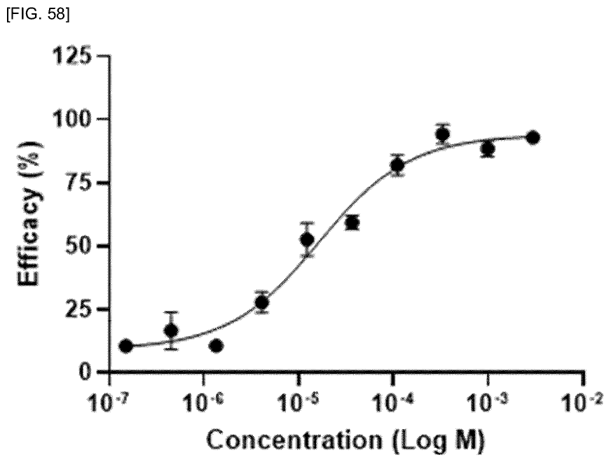

The endo-cannabinoid analogues SFO-EA and SFO-AP, produced according to the embodiments, exhibited an increase in binding efficiency to CB1 and CB2 in a concentration-dependent manner. The EC50 values were determined as follows: 512 nM for SFO-EA/CB1 (FIG. 55), 1017 nM for SFO-EA/CB2 (FIG. 56), 329 nM for SFO-AP/CB1 (FIG. 57), and 1632 nM for SFO-AP/CB2 (FIG. 58). Similar to the positive control, both analogues demonstrated a higher binding efficiency for CB1 than for CB2.

TABLE 27

| | | Result | Result | Graph | |
|---|---|---|---|---|---|
| Sample | Receptor | Type | Value (nM) | Min | Max |
| CP55940 | CB1 | EC50 | 0.222 | 6.4389 | 101.75 |
| | CB2 | EC50 | 1.019 | 5.4524 | 102.07 |
| SFO-EA | CB1 | EC50 | 511.691 | 9.5913 | 102.88 |
| | CB2 | EC50 | 1017.157 | 8.985 | 83.249 |
| SFO-AP | CB1 | EC50 | 329.454 | 5 | 107.12 |
| | CB2 | EC50 | 1632.091 | 9.1835 | 93.997 |

### 3-2. Evaluation of Growth Inhibitory Effects Against Harmful Microorganisms

To evaluate the growth inhibitory effects of the endo-cannabinoid analogues produced according to the embodiments of the present invention against harmful microorganisms, tests were conducted on five microorganisms specified in the microbial limit test method of the Ministry of Food and Drug Safety (MFDS): Staphylococcus aureus (Sa), Pseudomonas aeruginosa (Pa), Escherichia coli (Ec), Salmonella enteritidis (Se), and Candida albicans (Ca). To evaluate the growth inhibitory effects, each pre-cultured microorganism was inoculated into a culture medium at a concentration corresponding to an optical density (OD) of 0.1 at 600 nm. The cultures were supplemented with the endo-cannabinoid analogues SFO-EA and SFO-AP, produced according to the embodiments of the present invention, at concentrations ranging from 0 ppm to 10 ppm. During the incubation period, optical density (OD) at 600 nm was measured every 20 minutes using a microplate reader to assess cell growth, and the growth inhibitory effects were determined based thereon.

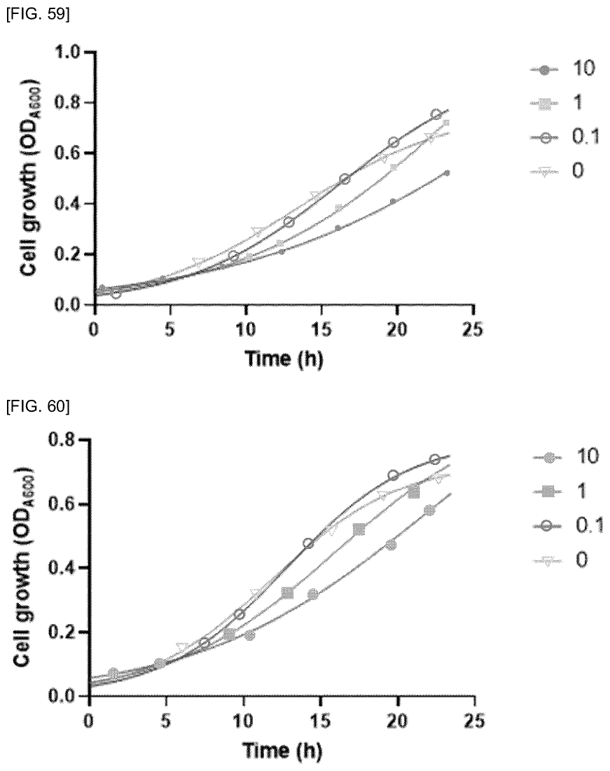

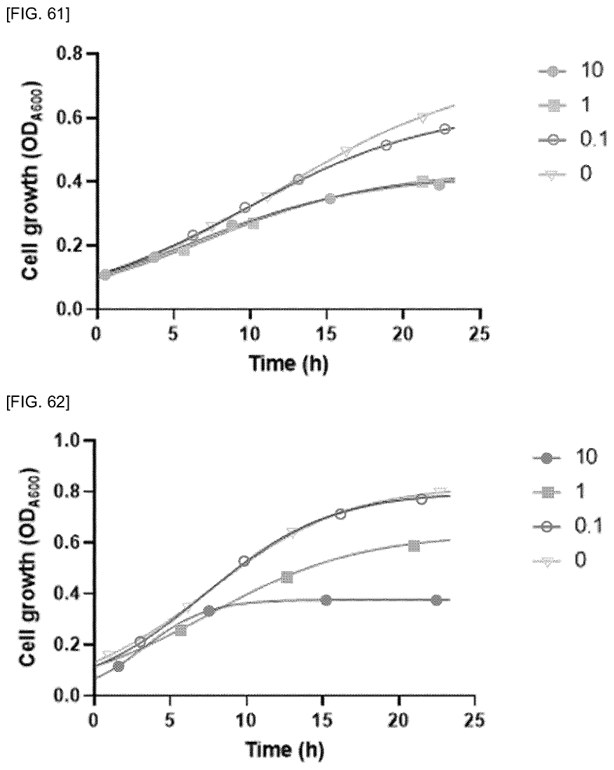

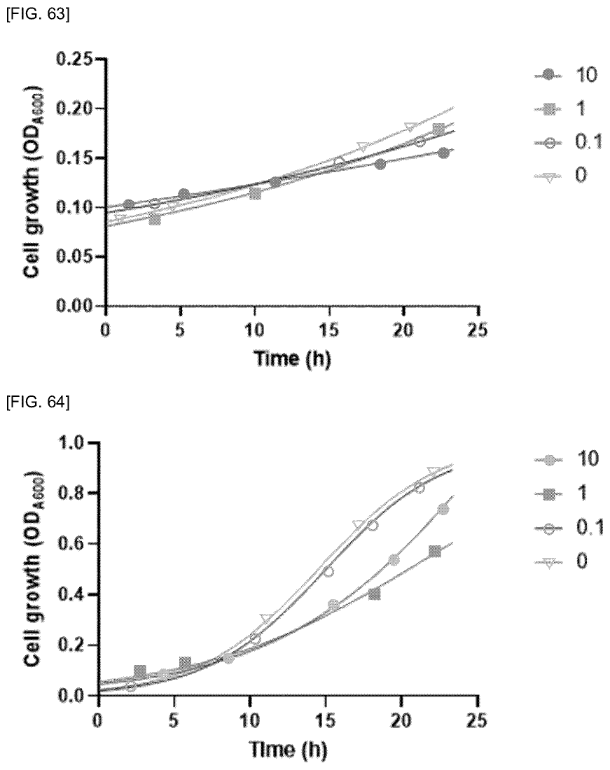

Upon analysis of the results, it was found that the addition of SFO-EA to the medium exhibited a growth inhibitory effect against Sa (FIG. 59), Ec (FIG. 60), Se (FIG. 61), and Ca (FIG. 62) at concentrations of 1 ppm or higher. However, no growth inhibitory effect was observed against Pa across the entire tested concentration range (FIG. 63).

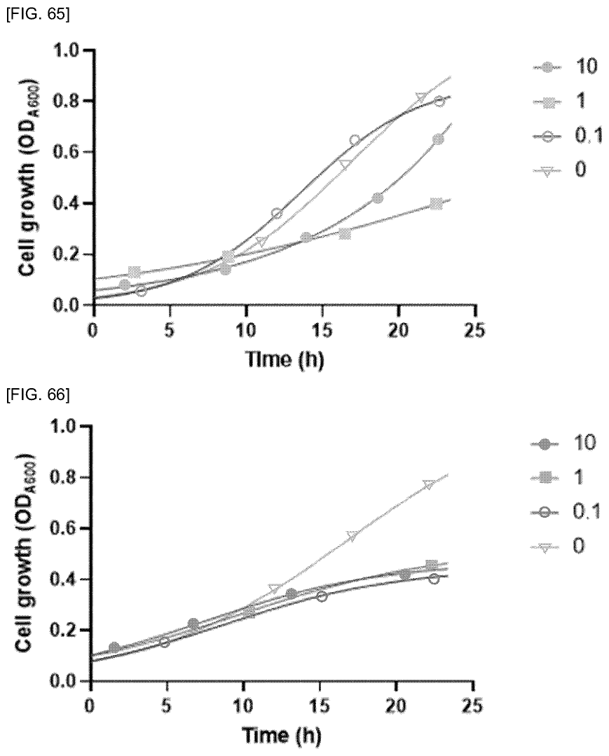

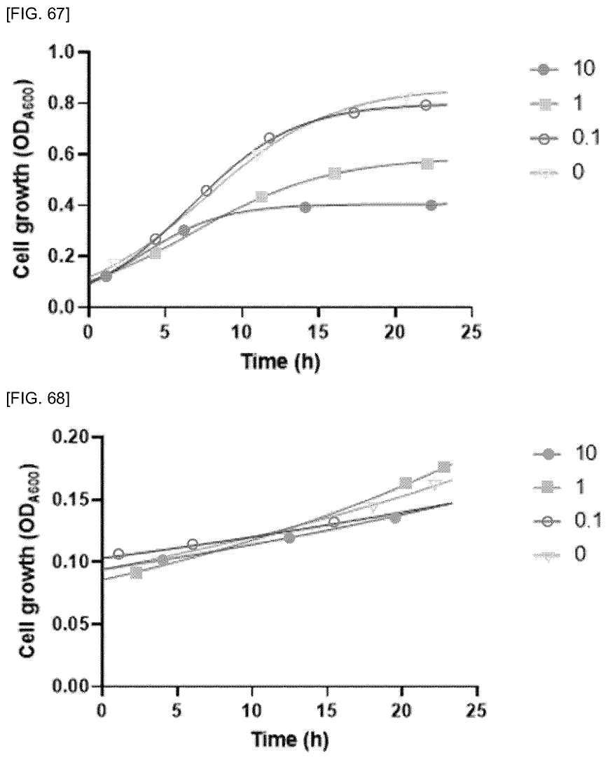

Meanwhile, the addition of SFO-AP to the medium exhibited a growth inhibitory effect against Sa (FIG. 64), Ec (FIG. 65), and Ca (FIG. 67) at concentrations of 1 ppm or higher, and against Se at 0.1 ppm or higher (FIG. 66). However, similar to SFO-EA, no growth inhibitory effect was observed against Pa across the entire tested concentration range (FIG. 68).

### 3-3. Evaluation of Cytotoxicity on Animal Cells

The cytotoxicity of the endo-cannabinoid analogues produced according to the embodiments of the present invention on animal cells was evaluated using the NIH/3T3 cell line, a mouse derived fibroblast, and the HaCaT cell line, a human-derived keratinocyte. Each cell line was cultured in an incubator containing 5% carbon dioxide at 37° C. using Dulbecco's Modified Eagle's Medium (DMEM) containing penicillin and streptomycin at concentrations of 100 IU/mL and 100 μg/mL, respectively, as well as 10% fetal bovine serum (FBS). Cytotoxicity was assessed using the MTT assay, which is based on the principle that 3-(4,5-dimethylthiazol-2-yl)-2,5-diphenyltetrazolium bromide (MTT), a yellow substrate, is reduced by dehydrogenase activity in viable mitochondria to form the purple product, 1-(4,5-dimethylthiazol-2-yl)-3,5-diphenylformazan (formazan).

The subcultured cells were seeded in a 96-well plate containing 100 μL of DMEM medium at a density of 1×10⁵ cells/well and cultured for 48 hours. Subsequently, the medium was replaced with serum-free media without FBS, and the prepared endo-cannabinoid analogues (SFO-EA, SFO-AP, SFO-MIPA, CAM-MIPA, and SFF-MIPA) were each added at concentrations ranging from 0.05 to 300 μM, followed by an additional 48-hour incubation. After the 48 hours of incubation, 10 μL of EZ-Cytox, an MTT assay solution from DoGenBio, was added to each well, followed by a 2-hour incubation. The absorbance was then measured at 450 nm to determine the number of viable cells, and the values were compared with those of the control group to analyze cytotoxicity. The obtained values were subjected to an unpaired t-test using GraphPad software to determine statistical significance compared to the control group.

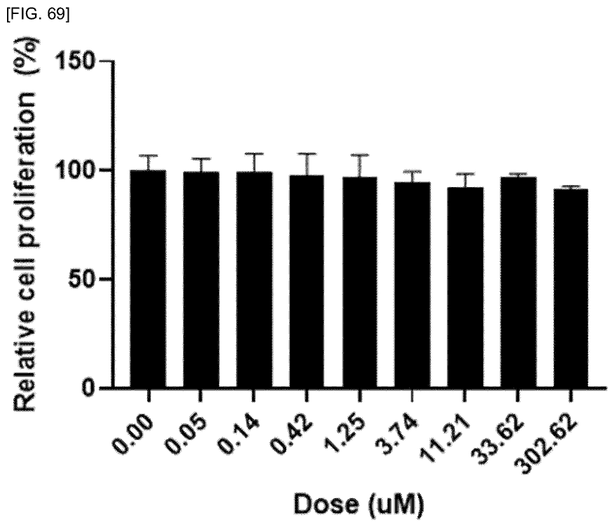

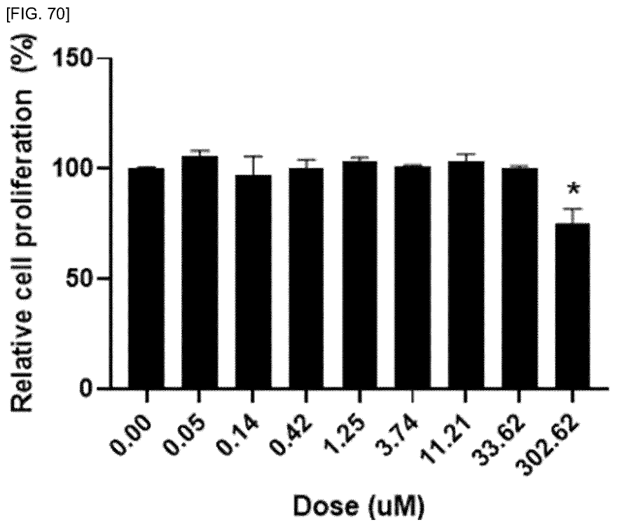

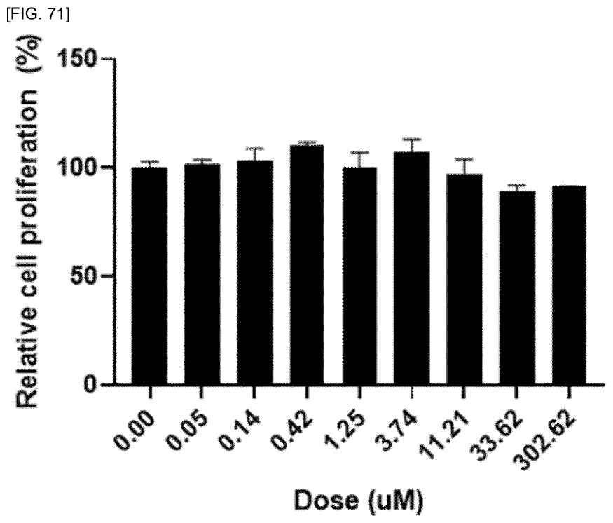

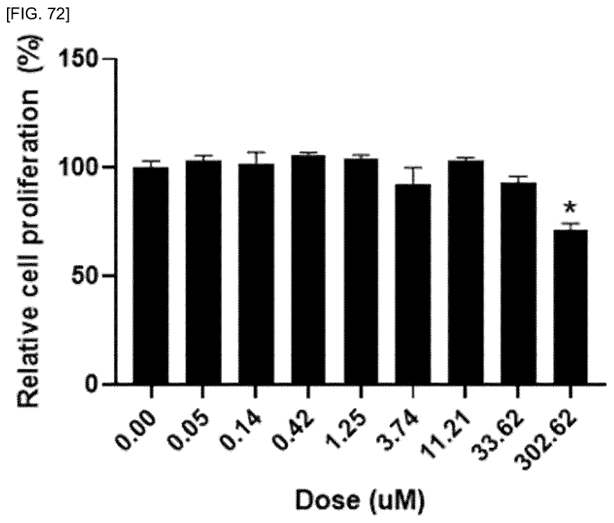

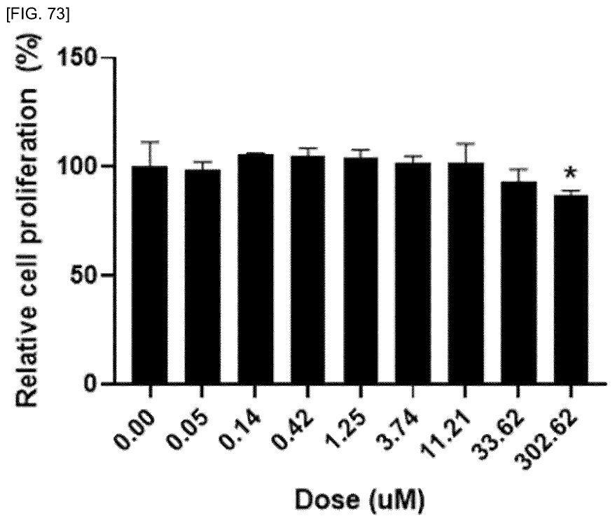

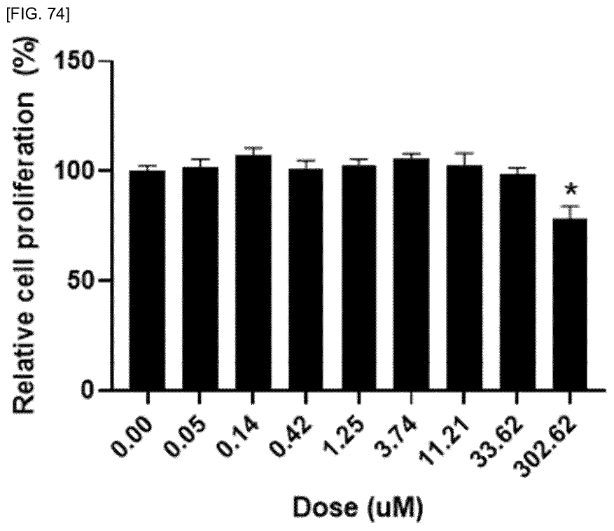

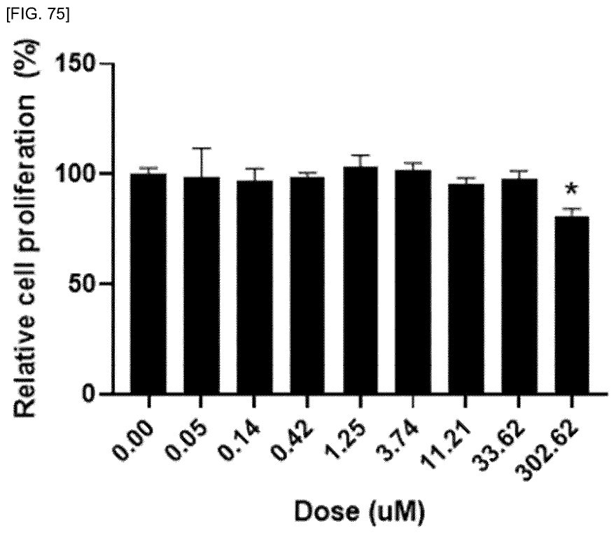

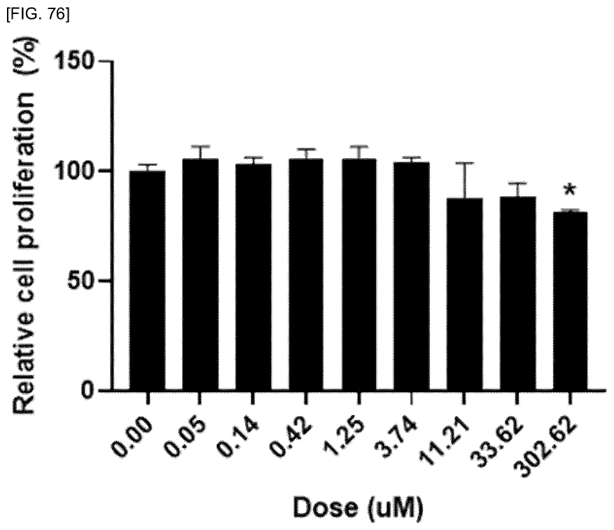

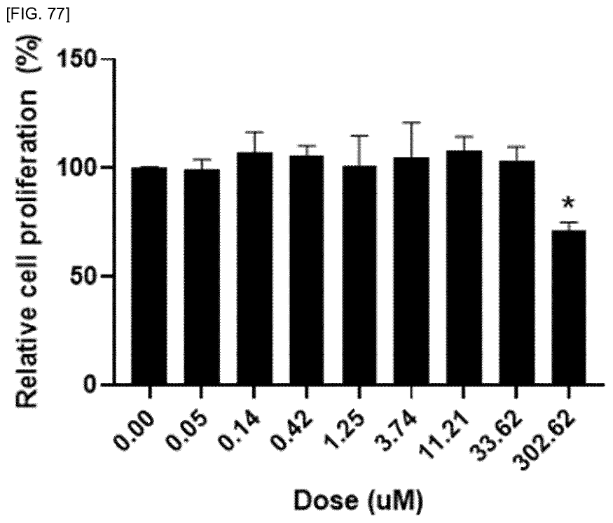

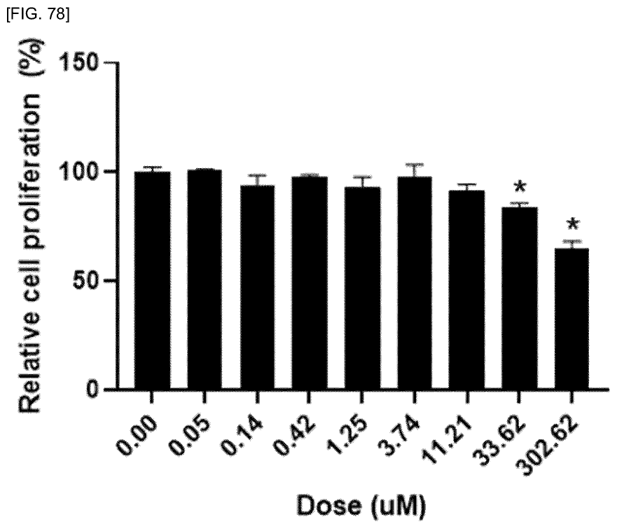

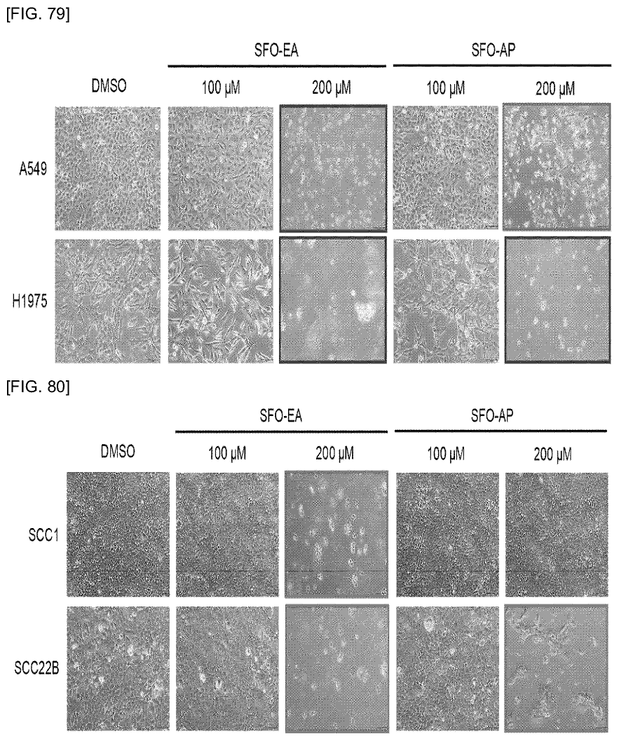

The experimental results demonstrated that SFO-EA did not exhibit cytotoxicity against 3T3 cells within the tested concentration range (FIG. 69). However, in HaCaT cells, cytotoxicity was observed at the highest concentration of 303 μM (FIG. 70). Similar to SFO-EA, SFO-AP did not exhibit cytotoxicity against 3T3 cells within the tested concentration range (FIG. 71). However, for HaCaT cells, cytotoxicity was observed at the highest concentration of 303 μM (FIG. 72). Meanwhile, SFO-MIPA exhibited cytotoxicity in both 3T3 and HaCaT cells at the highest concentration of 303 μM (FIGS. 73 and 74). Similarly, CAM-MIPA exhibited cytotoxicity in both 3T3 and HaCaT cells at the highest concentration of 303 μM (FIGS. 75 and 76). Additionally, SFF-MIPA exhibited cytotoxicity in both 3T3 and HaCaT cells at the highest concentration of 303 μM (FIG. 77). Furthermore, in HaCaT cells, cytotoxicity was observed at concentrations of 34 μM and higher (FIG. 78).

### 3-4. Evaluation of Cytotoxicity on Human Cancer Cells

The human lung cancer cell line A549 (ATCC catalog number: CCL-185) and the human non-small cell lung cancer (NSCLC) cell line H1975 (ATCC catalog number: CRL-5908) were obtained from ATCC (American Type Culture Collection). For cell culture, Dulbecco's Modified Eagle Medium (DMEM; Thermo Fisher Scientific, Catalog number: 11995-073) supplemented with 10% fetal bovine serum (FBS; Thermo Fisher Scientific, Catalog number: 10437-036) was used. Each cancer cell line (5×10⁵ cells) was seeded in a 6-well plate and cultured for 24 hours. After incubation, the cells were treated with DMSO (control group) or SFO-EA and SFO-AP dissolved in DMSO at concentrations of 100 μM and 200 μM, respectively, followed by an additional 48-hour incubation at 37° C. Cell death was then examined under a microscope (Nikon Eclipse TE2000-S) (FIG. 79). The experimental results demonstrated no significant changes in the 100 μM SFO-EA and SFO-AP treatment groups. However, in the 200 μM treatment groups, cell death was observed for both SFO-EA and SFO-AP. Notably, the cell killing effect was more pronounced in H1975 non-small cell lung cancer (NSCLC) cells.

Additionally, two human head and neck squamous carcinoma (HNSCC) cell lines, SCC-1 and SCC22B, were purchased from Sigma and cultured using DMEM medium supplemented with 10% FBS. Similar to the aforementioned lung cancer cells, the two HNSCC cell lines (SCC-1 and SCC22B, 5×10⁵ cells) were cultured, followed by treatment with SFO-EA or SFO-AP and additional incubation. Cell death was then examined under a microscope (Nikon Eclipse TE2000-S) (FIG. 80). Similar to the results observed in lung cancer cells, no significant changes were detected in oral cancer cells treated with 100 μM of SFO-EA or SFO-AP. However, in the 200 μM treatment groups, cell death was observed in response to both SFO-EA and SFO-AP.

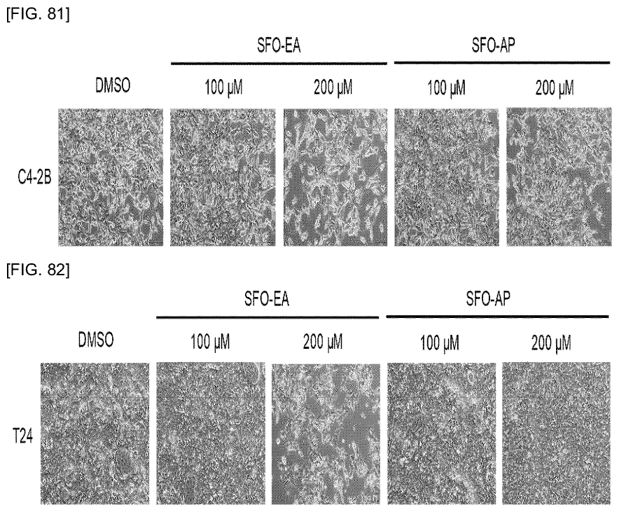

Furthermore, the human prostate cancer cell line C4-2B (CRL-3315) was purchased from ATCC and cultured using DMEM medium supplemented with 10% FBS. Similar to the aforementioned cancer cells, the two human prostate cancer cell lines (5×10⁵ cells) were cultured, followed by treatment with SFO-EA or SFO-AP and additional incubation. Cell death was then examined using a microscope (Nikon Eclipse TE2000-S) (FIG. 81). Similar to the results observed in the aforementioned cancer cells, no significant changes were detected in prostate cancer cells treated with 100 μM of SFO-EA or SFO-AP. However, in the 200 μM treatment groups, cell death was observed in response to both SFO-EA and SFO-AP.

Lastly, the human urinary bladder cancer cell line T24 (HTB-4) was purchased from ATCC and cultured using DMEM medium supplemented with 10% FBS. Similar to the aforementioned cancer cells, the two human urinary bladder cancer cell lines (5×10¹ cells) were cultured, followed by treatment with SFO-EA or SFO-AP and additional incubation. Cell death was then examined using a microscope (Nikon Eclipse TE2000-S) (FIG. 82). Similarly, no significant changes were detected in urinary bladder cancer cells treated with 100 μM of SFO-EA or SFO-AP. However, in the 200 μM treatment groups, cell death was observed in response to both SFO-EA and SFO-AP.

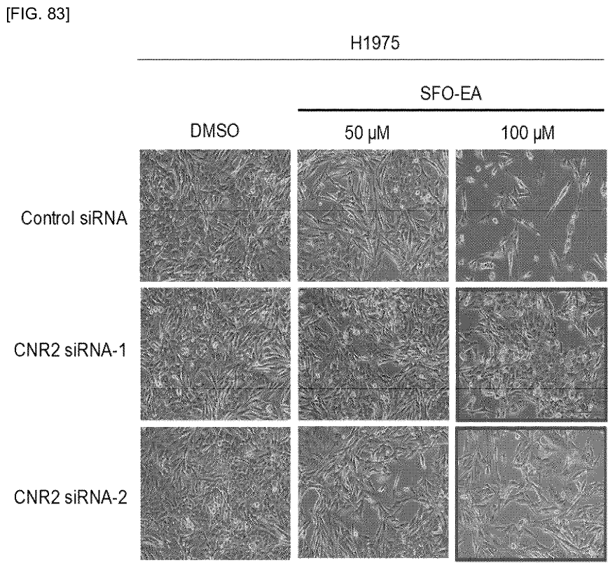

Although the extent of cell death varied, a cytotoxic effect was observed in most of the tested cancer cells following treatment with 100 μM to 200 μM. Therefore, to determine whether the cancer cell killing effect of these endo-cannabinoid analogues (SFO-EA and SFO-AP) is mediated through cannabinoid receptor signaling and the subsequent induction of apoptosis, two CB2 (CNR2) siRNAs (5′-CUCAUCAACUCCAUGGUCA[dT][dT]-3′ and 5′-CUGUUCAUCGCCUUCCUCU[dT][dT]-3′) and a control siRNA (Universal Negative Control #1, SIC001) were purchased from Sigma. The human non-small cell lung cancer cell line H1975 (5×10⁵ cells) was seeded in a 60 mm tissue culture plate and cultured for 24 hours. Subsequently, the cells were transfected with 100 nM of each prepared siRNA using Lipofectamine RNAiMAX reagent (Thermo Fisher Scientific, Cat #13778150) and Reduced Serum Medium (Opti-MEM™, TFS, Catalog #31985070) for 48 hours. Subsequently, DMSO and SFO-EA were added at concentrations of 50 μM and 100 μM, and the differences were compared (FIG. 83). The experimental results confirmed that treatments with the two types of siRNA significantly attenuated the cell killing effect of SFO-EA. These results indicate that the endo-cannabinoid analogues prepared according to the embodiments of the present invention mediate intracellular signaling through human cannabinoid receptors, thereby inducing cancer cell death.

### 3-5. Evaluation of Anti-Inflammatory Effects of Endo-Cannabinoid Analogues (Inhibition of NO Production)

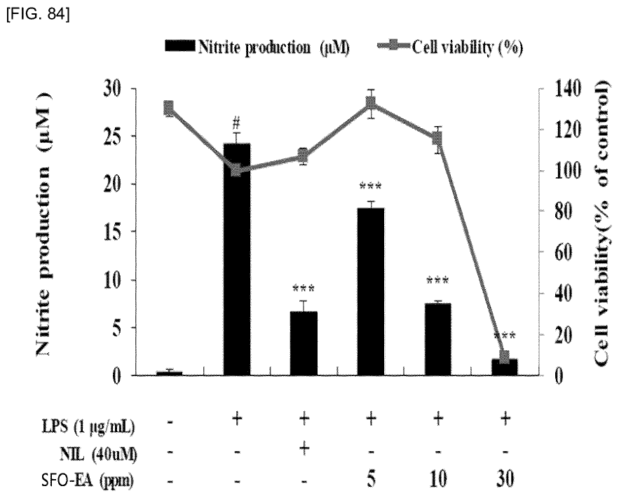

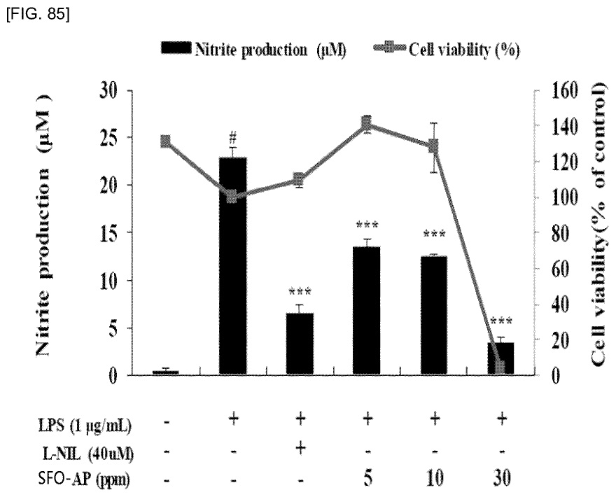

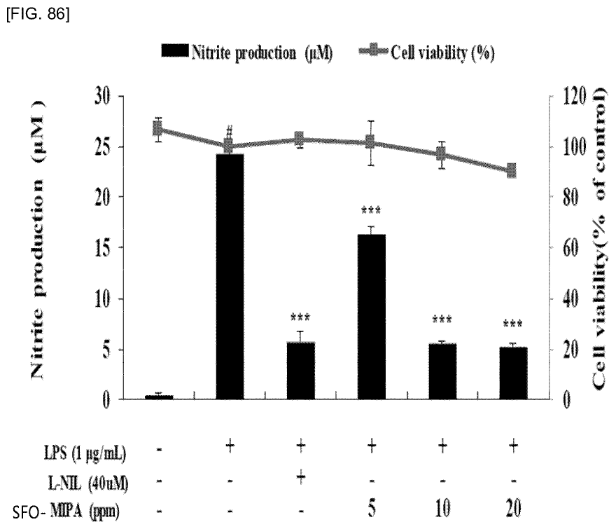

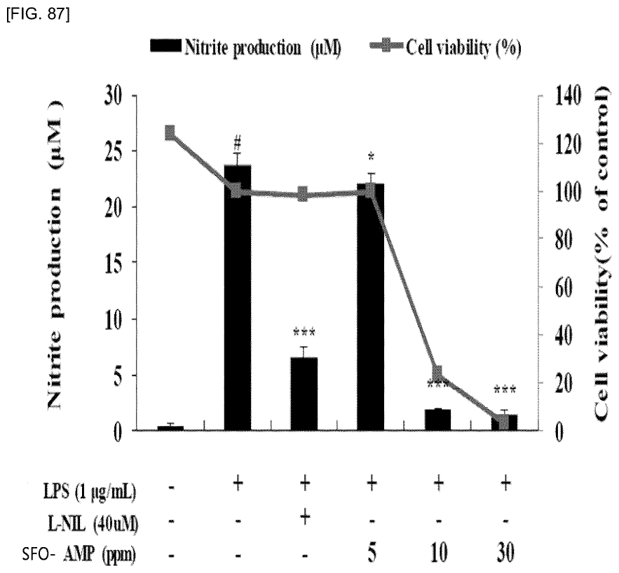

The anti-inflammatory effect was evaluated using four endo-cannabinoid analogues (SFO-EA, SFO-AP, SFO-MIPA, and SFO-AMP) prepared from SFO according to the embodiments of the present invention. Inflammatory factor production was induced by treating macrophages with lipopolysaccharides (LPS), and the four endo-cannabinoid analogues prepared according to the embodiments of the present invention were administered to determine whether they inhibit the inflammation factor production. Specifically, RAW 264.7 cells were purchased from the Korean Cell Line Bank (KCLB) and cultured using DMEM medium supplemented with antibiotics (penicillin/streptomycin) and FBS. The expression level of NO₂⁻ present in the cell culture supernatant was measured using the Griess reagent. For cytotoxicity evaluation, 3-(4,5-dimethylthiazol-2-yl)-2,5-diphenyltetrazolium bromide (MTT) and dimethyl sulfoxide (DMSO) were used. For NO content measurement, LPS and the Griess reagent were used. Absorbance was measured using a microplate reader, followed by an anti-inflammatory efficacy evaluation. RAW 264.7 cells were dispensed into a 24-well plate at a density of 1.5×10⁵ cells/well. After a 24-hour pre-incubation, the samples were administered at various concentrations and further incubated for 24 hours. After 24 hours, 100 μL of the supernatant was collected from the 24-well plate and transferred to a 96-well plate. Then, 100 μL of the Griess reagent was added and incubated for 10 minutes, followed by absorbance measurement at 540 nm. To evaluate the extent to which the samples inhibit NO production in LPS-induced RAW 264.7 cells, the Griess reagent was used to measure the amount of NO accumulated in the cell culture medium in the form of nitrite. The experimental results demonstrated that SFO-EA (FIG. 84), SFO-AP (FIG. 85), SFO-MIPA (FIG. 86), and SFO-AMP (FIG. 87) exhibited a concentration-dependent inhibitory effect on NO production at 5 ppm (14 μM), 10 ppm (28 μM), and 20 ppm (56 μM). Furthermore, these samples demonstrated a superior anti-inflammatory effect compared to L-NIL (40 μM), which was used as a positive control. In particular, it was found that SFO-MIPA exhibited the strongest anti-inflammatory activity, whereas SFO-AMP demonstrated potent anti-inflammatory activity along with some degree of cytotoxicity.

### 3-6. Evaluation of Anti-Inflammatory Effects of Endo-Cannabinoid Analogues (Inhibition of Pro-Inflammatory Cytokine Production)

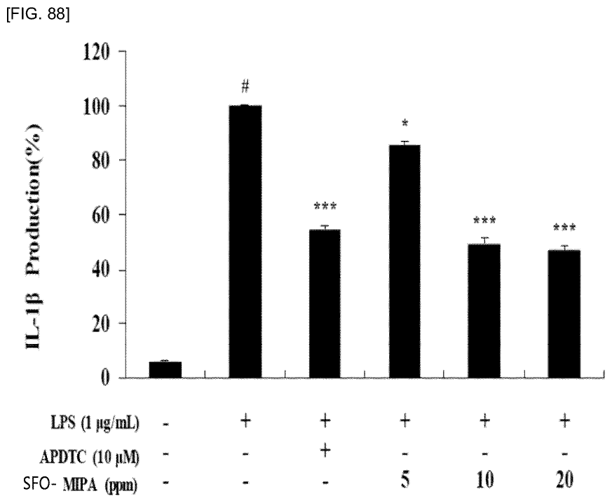

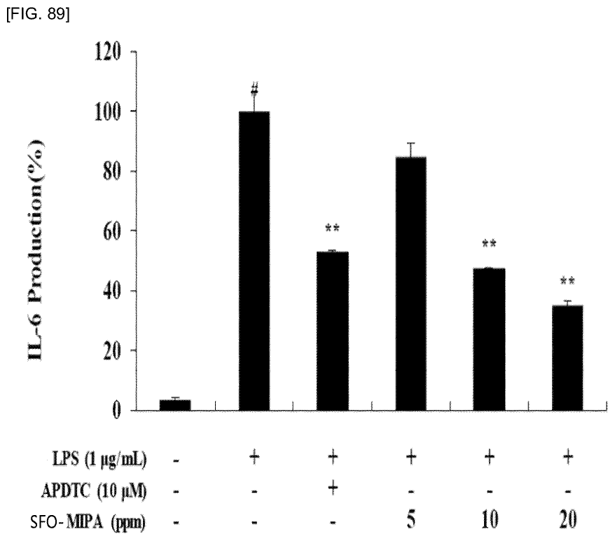

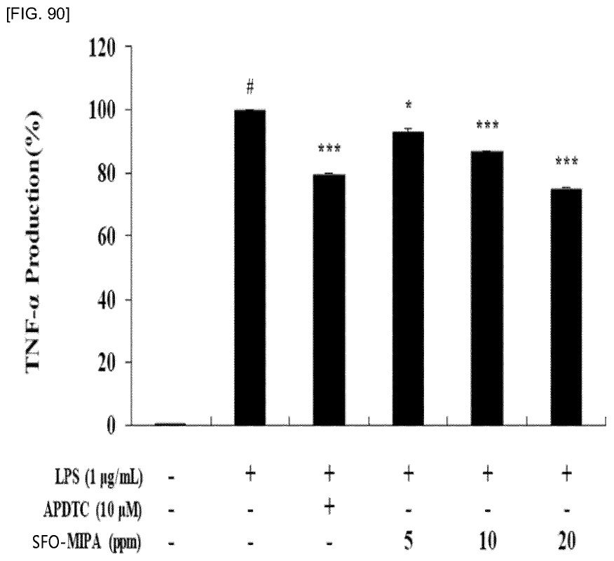

To investigate whether SFO-MIPA, which exhibited strong anti-inflammatory effects in Example 3-5, inhibits the production of pro-inflammatory cytokines (IL-1β, IL-6, and TNF-α), the changes in pro-inflammatory cytokine levels were measured using an ELISA kit. RAW 264.7 cells were dispensed into a 24-well plate at a density of 1.5×10¹ cells/well, incubated for 24 hours, and then treated with a non-cytotoxic concentration of the sample and LPS (1 pg/mL), followed by an additional 24-hour incubation. The cell culture was centrifuged at 5,000 rpm for 5 minutes, and the supernatant was collected. The levels of IL-13 (FIG. 88), IL-6 (FIG. 89), and TNF-α (FIG. 90) were then measured using an ELISA kit according to the manufacturer's instructions. The experimental results demonstrated that SFO-MIPA significantly reduced the production of pro-inflammatory cytokines induced by LPS. The NF-κB-specific inhibitor APDTC decreased the production of IL-1β, IL-6, and TNF-α by 45.39%, 47.12%, and 20.28%, respectively. In contrast, the sample SFO-MIPA decreased the production of IL-1β, IL-6, and TNF-α by 53.15%, 65.06%, and 24.62%, respectively, at the highest concentration of 20 ppm.

### 3-7. Evaluation of Anti-Asthmatic Effects of Endo-Cannabinoid Analogues

As confirmed in Examples 3-5 and 3-6, the endo-cannabinoid analogues prepared according to the embodiments of the present invention exhibited anti-inflammatory effects. To evaluate the applicability of these compounds as therapeutic agents for asthma, which is a representative inflammatory disease, anti-asthmatic effects were assessed using an asthma mouse model. Six-week-old female Balb/c mice with an average body weight of approximately 20 g were subjected to a one-week acclimation period. Following acclimation, the mice were sensitized intraperitoneally with 200 μL of phosphate-buffered solution (PBS) containing a suspension of aluminum hydroxide (Alum; A8222, Sigma) and ovalbumin (OVA; A5503, Sigma) on Day 0 (30 μg OVA/30 mg Alum) and Day 7 (30 μg OVA/30 mg Alum). For three consecutive days, from Day 11 to Day 13, the mice were exposed to 1% ovalbumin by inhalation for 60 minutes using an ultrasonic nebulizer. The positive control and SFO-EA were administered from Day 9 to Day 13. On Day 15, a bronchial incision was performed, followed by bronchoalveolar lavage (BAL) using a total of 1.4 mL of saline and the samples were collected.

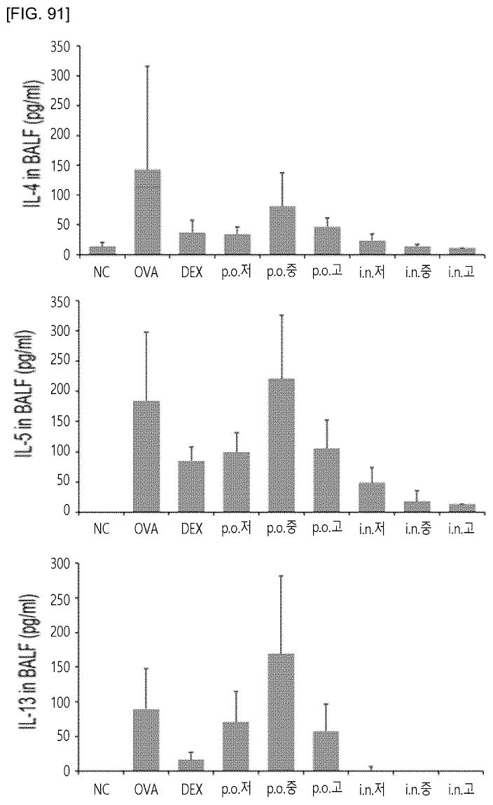

The following groups were prepared: a normal control group (NC) that was neither administered nor exposed to ovalbumin; an asthma-induced group (OVA) that was administered and exposed to ovalbumin to induce bronchial asthma; a comparison group that received an oral dose of dexamethasone (DEX, 1 mg/kg, p.o.) one hour before ovalbumin inhalation; and groups that were administered the endo-cannabinoid analogues prepared according to the embodiments of the present invention either orally (p.o.) or intranasally (i.n.) at concentrations of 0.625%, 1.25%, or 2.5%. A sandwich-type immunoenzymatic reaction method was used to measure Th2 cytokines present in the bronchial alveoli. The bronchoalveolar lavage fluid obtained from each experimental group was added to a 96-well plate coated with cytokine antibodies and incubated at room temperature for 2 hours to induce an antigen-antibody reaction. An ELISA kit (R&D Systems) was used to measure the levels of IL-4, IL-5, and IL-1β, and the concentrations of each cytokine were determined according to the manufacturer's instructions. As shown in FIG. 91, the oral administration of the endo-cannabinoid analogues prepared according to the embodiments of the present invention exhibited an IL-4 and IL-5 production inhibitory effect (0.625% and 2.5%) in mice. In contrast, intranasal administration demonstrated an overall superior inhibitory effect on the production of IL-4, IL-5, and IL-13.

### 3-8. Evaluation of Pain-Suppressing Effects of Endo-Cannabinoid Analogues

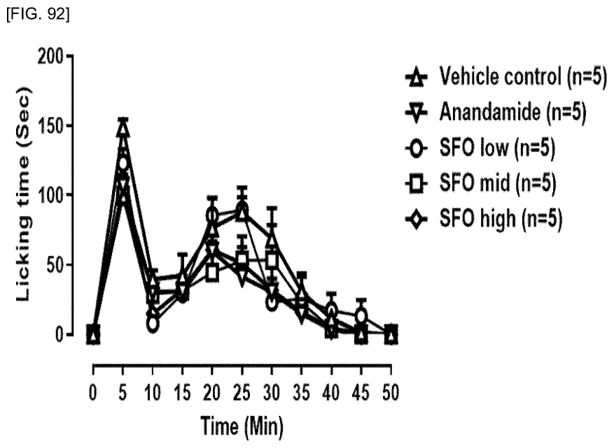

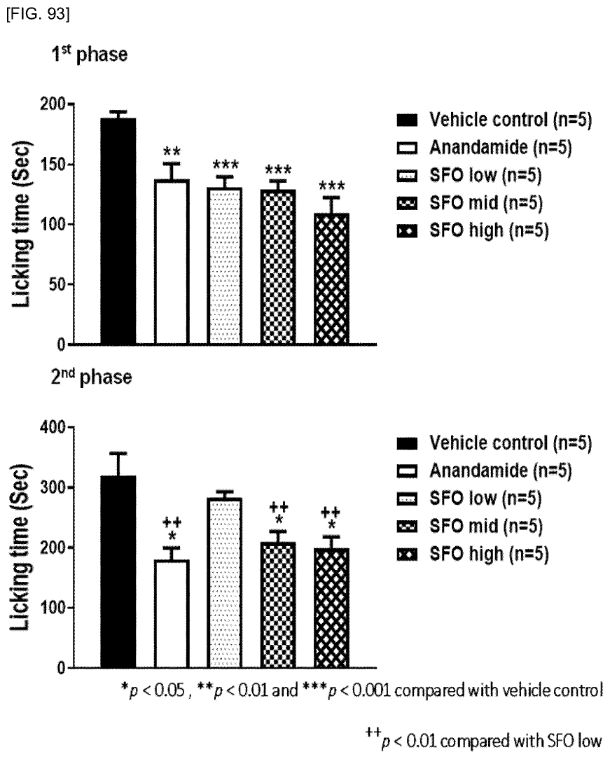

To evaluate the pain-relieving effect of the endo-cannabinoid analogue (SFO-EA) derived from sunflower oil, male ICR mice aged 5 to 6 weeks and weighing approximately 25 g were used. The pain-relieving efficacy of SFO-EA was evaluated by analyzing the pain responses in a formalin test, which is a well-known acute inflammatory pain mouse model. The formalin test is a model that induces acute inflammatory pain by injecting a formaldehyde solution into the mouse paw. In the present invention, a 2.5% formaldehyde solution was injected at a volume of 20 μL. In particular, the positive control group was administered anandamide (0.1 μg/20 μL), a known endogenous antagonist of the CB1 receptor. To evaluate the dose-dependent effect of SFO-EA, it was administered at 0.1 μg/20 μL (low), 0.3 μg/20 μL (mid), and 1 μg/20 μL (high). Each test substance was administered into the mouse paw 20 minutes prior to formalin injection. After the formalin injection, the duration of chewing, biting, and licking responses of the injected paw was measured at 5-minute intervals over a total period of 1 hour to analyze the licking time, which indicates pain perception. The results are presented in FIGS. 92 and 93.

The experimental results demonstrated that mice administered SFO-EA at mid and high concentrations exhibited a reduction in pain perception compared to those with acute inflammatory pain induction. Additionally, the degree of pain relief was found to be comparable to that observed in mice treated with anandamide, which was used as the positive control. This suggests that SFO-EA exhibits a pain-relieving effect.

### 3-9. Evaluation of Effects of Endo-Cannabinoid Analogues on Weight Gain in Animals

To evaluate the effect of the endo-cannabinoid SFO-EA on weight gain in animals, weaned piglets were fed a diet supplemented with 100 mg of SFO-EA per kg of body weight for three weeks. Using 10 weaned piglets per group (control and experimental), the initial and final body weights were measured twice to determine the average weight gain and feed intake. The feed conversion ratio (FCR) was then calculated based on these measurements (Table 28).

TABLE 28

| | | Control group | | | Experimental Group | | |
|---|---|---|---|---|---|---|---|
| Day | | H1-21 | H1-31 | Mean | H1-1 | H1-11 | Mean |
| Initial Body | 1 | 7.1 | 5.6 | | 7.1 | 5.6 | |
| Weight | 2 | 6.6 | 5.6 | | 6.8 | 5.5 | |
| | 3 | 6.5 | 5.5 | | 6.4 | 5.5 | |
| | 4 | 6.2 | 5.5 | | 6.4 | 5.5 | |
| | 5 | 6.0 | 5.4 | | 6.3 | 5.4 | |
| | 6 | 6.0 | 5.4 | | 6.1 | 5.1 | |
| | 7 | 6.0 | 5.1 | | 6.0 | 5.1 | |
| | 8 | 5.9 | 4.9 | | 6.0 | 5.1 | |
| | 9 | 5.8 | 4.9 | | 5.9 | | |
| | 10 | | 4.7 | | 5.8 | | |
| | Mean | 6.23 | 5.26 | 5.75 | 6.28 | 5.35 | 5.82 |
| Final Body | 1 | 13.0 | 7.9 | | 10.6 | 12.4 | |
| Weight | 2 | 9.8 | 8.9 | | 11.1 | 11.0 | |
| | 3 | 9.8 | 7.9 | | 13.5 | 12.0 | |
| | 4 | 8.6 | 9.3 | | 13.1 | 10.0 | |
| | 5 | 10.4 | 9.9 | | 12.3 | 10.0 | |
| | 6 | 8.3 | 9.5 | | 11.3 | 8.9 | |
| | 7 | 10.7 | 10.6 | | 10.3 | 11.8 | |
| | 8 | 9.6 | 7.6 | | 9.3 | 8.7 | |
| | 9 | 14.1 | 10.5 | | 8.7 | | |
| | 10 | | 10.6 | | 10.5 | | |
| | Mean | 10.48 | 9.27 | 9.87 | 11.07 | 10.60 | 10.84 |
| | Initial | 0.42 | 0.33 | 0.38 | 0.41 | 0.21 | 0.31 |
| | Standard | | | | | | |
| | Deviation | | | | | | |
| | Final | 1.92 | 1.16 | 1.54 | 1.54 | 1.42 | 1.48 |
| | Standard | | | | | | |
| | Deviation | | | | | | |
| | No. of | 9 | 10 | 9.5 | 10 | 8 | 9 |
| | Animals | | | | | | |
| | Days of | 21 | 21 | 21 | 21 | 21 | 21 |
| | Feed Intake | | | | | | |
| | | 70.0 | 70.0 | 140.0 | 70.0 | 70.0 | 140.0 |
| | Total Feed | 7.78 | 7.00 | 7.39 | 7.00 | 8.75 | 7.88 |
| | Intake | | | | | | |
| | (/animal) | | | | | | |
| | Daily | 0.37 | 0.333 | 0.35 | 0.333 | 0.417 | 0.38 |
| | Feed Intake | | | | | | |
| | (animal/day) | | | | | | |
| Weight Gain | Total | 4.24 | 4.01 | 4.13 | 4.79 | 5.25 | 5.02 |
| (kg) | Weight Gain | | | | | | |
| | (/animal) | | | | | | |
| | Daily | 0.202 | 0.191 | 0.20 | 0.228 | 0.250 | 0.24 |
| | Weight Gain | | | | | | |
| | (animal/day) | | | | | | |

| | | | | | | |
|---|---|---|---|---|---|---|
| Feed Conversion Rate | 1.832 | 1.746 | 1.79 | 1.461 | 1.667 | 1.56 |

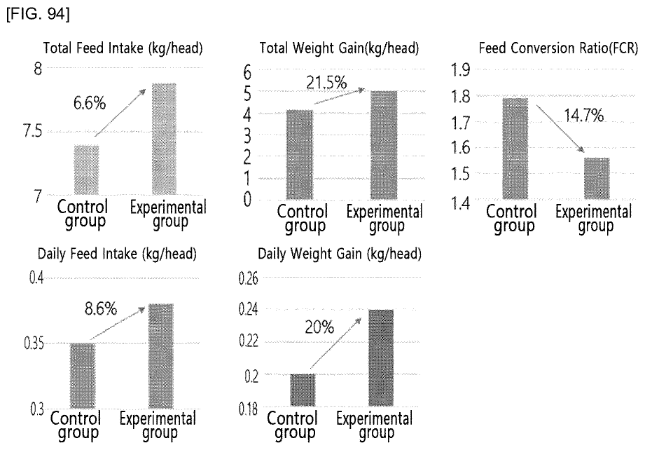

As summarized in FIG. 94, the analysis showed that the experimental group exhibited a 21.5% increase in weight gain, while the feed conversion ratio (FCR) decreased by 14.7% compared to the control group. These results suggest that the endo-cannabinoid analogue SFO-EA promoted weight gain and demonstrated its efficacy as a feed additive.

### 3-10. Toxicity Analysis of Oral Administration of Endo-Cannabinoid Analogues

A single-dose oral toxicity study was conducted by BioToxTech Co., Ltd. to analyze the toxicity of four endo-cannabinoid analogues (SFO-EA, SFO-AP, SFO-MIPA, and SFO-AMP) derived from sunflower oil following oral administration. Specifically, male and female ICR mice were used to observe toxic responses following a single oral administration of the test substances SFO-EA, SFO-AP, SFO-MIPA, and SFO-AMP at a dose of 5,000 mg/kg.

The administration volume for each subject was calculated based on the pre-administration (post-fasting) body weight on the day of administration. The preparation was melted using a water bath at a temperature below 50° C. before use. A disposable syringe equipped with an oral gavage tube was used for forced intragastric administration. All animals were fasted for approximately 16 hours before administration while being allowed free access to water, and feed was provided approximately 4 hours after the final administration. On the day of administration (Day 1), general conditions (types of toxic signs, time of onset, recovery time, etc.) and mortality were monitored at least once within 30 minutes and at 1, 2, 4, and 6 hours post-administration. Subsequently, general symptoms were monitored once a day for three days (Day 2 to Day 4).

The experimental results showed that no mortality was observed following the administration of four endo-cannabinoid analogues (SFO-EA, SFO-AP, SFO-MIPA, and SFO-AMP) at a dose of 5,000 mg/kg. Body weight changes are presented in Tables 29 to 32. As a result, no significant toxicity was observed upon oral administration of the four endo-cannabinoid analogues (SFO-EA, SFO-AP, SFO-MIPA, and SFO-AMP).

TABLE 29

| | | |
|---|---|---|
| Dosage | Animal | Weight (g) |

| (mg/kg) | Gender | ID | Day 1 | Day 2 | Day 4 |
|---|---|---|---|---|---|
| 5.000 | Male | 1101 | 204.0 | 209.5 | 244.6 |
| | Female | 2101 | 135.9 | 147.2 | 166.1 |

TABLE 30

| | | |
|---|---|---|
| Dosage | Animal | Weight (g) |

| (mg/kg) | Gender | ID | Day 1 | Day 2 | Day 4 |
|---|---|---|---|---|---|
| 5.000 | Male | 1101 | 175.6 | 192.1 | 222.6 |
| | Female | 2101 | 134.7 | 148.2 | 161.1 |

TABLE 31

| | | |
|---|---|---|
| Dosage | Animal | Weight (g) |

| (mg/kg) | Gender | ID | Day 1 | Day 2 | Day 4 |
|---|---|---|---|---|---|
| 5.000 | Male | 1101 | 217.5 | 209.6 | 246.1 |
| | Female | 2101 | 134.8 | 142.4 | 161.2 |

TABLE 32

| | | |
|---|---|---|
| Dosage | Animal | Weight (g) |

| (mg/kg) | Gender | ID | Day 1 | Day 2 | Day 4 |
|---|---|---|---|---|---|
| 5.000 | Male | 1101 | 171.4 | 183.8 | 210.7 |
| | Female | 2101 | 169.8 | 173.9 | 201.1 |

From the above description, those skilled in the art to which the present invention pertains would understand that the present invention can be implemented in other specific forms without changing its technical concept or essential features. In this regard, the above-described examples should be understood as illustrative and not limited in any way. The scope of the present invention should be interpreted to include all modifications or modified forms derived from the meaning and scope of the claims as set forth below, as well as equivalent concepts, rather than from the detailed description above.

1. A method for preparing endo-cannabinoid analogues, comprising performing an amidation reaction of a vegetable oil, which comprises triglycerides that provide two or more types of fatty acids through hydrolysis, with a C₂₋₆ aminoalcohol using lipase.

2. The method of claim 1,
wherein the endo-cannabinoid analogue is a fatty acid amide (FAA) provided by forming an amide bond between the carboxyl group of a fatty acid and the amine group of the C₂₋₆ aminoalcohol.

3. The method of claim 1,
wherein the vegetable oil is one or more selected from the group consisting of sunflower oil (SFO), olive oil (OLO), coconut oil (CNO), soybean oil (SBO), grapeseed oil (GSO), evening primrose oil (EPO), rapeseed oil (RSO), meadowfoam seed oil (MFO), abyssinica seed oil (ABSO), hempseed oil (HSO), avocado oil (AVO), rice bran oil (RBO), shea butter (SHB), Aurantiochytrium oil (ATO), camellia oil (CAM), safflower oil (SFF), palm oil, macadamia nut oil, mustard oil, and castor oil.

4. The method of claim 1,
wherein the two or more fatty acids are a mixture of two or more selected from the group consisting of linoleic acid, linolenic acid, oleic acid, stearic acid, palmitic acid, myristic acid, lauric acid, capric acid, eicosenoic acid, erucic acid, docosadienoic acid, behenic acid, docosahexaenoic acid, eicosapentaenoic acid, arachidonic acid, dodecanoic acid, hexanoic acid, caproic acid, tripalmitin, tricaproin, and tricaprin.

5. The method of claim 1,
wherein the C₂₋₆ aminoalcohol is one or more selected from the group consisting of 3-amino-1-propanol (AP), ethanolamine (EA), monoisopropanolamine (MIPA), 2-amino-2-methyl-1-propanol (AMP), 3-amino-1,2-propandiol, and serinol (2-aminopropane-1,3-diol).

6. The method of claim 1,
wherein the lipase is added in a ratio of 0.01% to 30% (w/v) relative to the vegetable oil.

7. The method of claim 1,
wherein the C₂₋₆ aminoalcohol is used in a molar ratio of 1:1 to 1:3 relative to the vegetable oil.

8. The method of claim 1,
wherein the amidation is performed at 40° C. to 70° C. for 15 to 200 hours.

9. The method of claim 1,
wherein the method is performed at a conversion of 85% to 99%.

10. A composition for inhibiting growth of harmful bacteria, comprising a vegetable oil-derived endo-cannabinoid analogues prepared by the method of claim 1 as an active ingredient.

11. The composition of claim 10,
wherein the harmful bacteria are Staphylococcus aureus, Escherichia coli, Salmonella, or Candida albicans.

12. A pharmaceutical composition for preventing or treating cancer, comprising a vegetable oil-derived endo-cannabinoid analogues prepared by the method of claim 1 as an active ingredient.

13. The pharmaceutical composition of claim 12,
wherein the cancer is lung cancer, oral cancer, prostate cancer, bladder cancer, colorectal cancer, colon cancer, cholangiocarcinoma (bile duct carcinoma), gastric cancer, breast cancer, pancreatic cancer, ovarian cancer, placental cancer, choriocarcinoma, esophageal squamous cell cancer, glioblastomas, melanomas, renal carcinomas, cervical squamous cell carcinomas, hepatocellular carcinomas, or cervical intraepithelial neoplasia.

14. A pharmaceutical composition for preventing or treating an inflammatory disease, comprising a vegetable oil-derived endo-cannabinoid analogues prepared by the method of claim 1 as an active ingredient.

15. The pharmaceutical composition of claim 14,
wherein the inflammatory disease is asthma, arthritis, atopic dermatitis, psoriasis, multiple sclerosis, ssRNA and dsRNA virus infections, sepsis, polychondritis, scleroderma, eczema, gout, periodontal disease, Behcet's syndrome, edema, vasculitis, Kawasaki disease, diabetic retinitis, autoimmune pancreatitis, angitis, glomerulonephritis, acute and chronic bronchitis, Crohn's disease, influenza infection, allergic asthma, allergic rhinitis, allergic mucositis, urticaria, anaphylaxis, systemic sclerosis, dermatomyositis, or inclusion body myositis.

16. A composition for suppressing pain, comprising a vegetable oil-derived endo-cannabinoid analogues prepared by the method of claim 1 as an active ingredient.

17. A composition selected from the group consisting of a feed composition, a food composition and a cosmetic composition, comprising a vegetable oil-derived endo-cannabinoid analogues prepared by the method of claim 1 as an active ingredient.

18. (canceled)

19. A method for preventing or treating cancer, comprising administering to a subject in need thereof the pharmaceutical composition according to claim 12.

20. A method for preventing or treating an inflammatory disease, comprising administering to a subject in need thereof the pharmaceutical composition according to claim 14.

21. A method for suppressing pain, comprising administering to a subject in need thereof the pharmaceutical composition according to claim 16.
